(19) United States
(12) Patent Application Publication
(10) Pub. No.: US 2023/0183720 A1
(43) Pub. Date: Jun. 15, 2023

(54) ENGINEERED PROTEIN DELIVERY PLATFORM AND USE THEREOF FOR ANTIBACTERIAL TREATMENTS

(71) Applicant: RAMOT AT TEL AVIV UNIVERSITY LTD., Tel Aviv (IL)

(72) Inventor: Dor Samuel SALOMON, Tel Aviv (IL)

(21) Appl. No.: 18/110,934

(22) Filed: Feb. 17, 2023

Related U.S. Application Data
(63) Continuation of application No. PCT/IL2021/051006, filed on Aug. 18, 2021.
(60) Provisional application No. 63/073,518, filed on Sep. 2, 2020.
(60) Provisional application No. 63/183,227, filed on May 3, 2021.

(51) Int. Cl. C12N 15/74; C12N 1/20; C07K 14/28; A61K 35/741; A61P 31/04

(52) U.S. Cl. CPC C12N 15/74; C12N 1/20; C07K 14/28; A61K 35/741; A61P 31/04; C12R 2001/63

(57) ABSTRACT

Provided herein is a non-pathogenic bacterium comprising a gene cassette encoding an antibacterial protein delivery platform, wherein the gene cassette is operably linked to a positive regulator inducibly-expressed from a genomic location.

### CROSS REFERENCE TO RELATED APPLICATIONS

This application is a Bypass Continuation of PCT Patent Application No. PCT/IL2021/051006 having International filing date of Aug. 18, 2021, which claims the benefit of priority of U.S. Provisional Patent Application No. 63/073,518, filed Sep. 2, 2020 and the benefit of priority of U.S. Provisional Patent Application No. 63/183,227, filed May 3, 2021, the contents of which are all incorporated herein by reference in their entirety.

### REFERENCE TO AN ELECTRONIC SEQUENCE LISTING

The contents of the electronic sequence listing (sequencelisting.xml; Size: 154,148 bytes; and Date of Creation: Feb. 16, 2023) is herein incorporated by reference in its entirety.

### FIELD OF THE INVENTION

Provided herein is a genetically engineered non-pathogenic bacterium comprising a gene cluster encoding an antibacterial protein delivery platform, wherein the gene cluster is operably linked to an inducibly-expressed positive regulation system.

### BACKGROUND

Antibiotics are manufactured at an enormous scale (about 100,000 tons annually worldwide), and their use had a great beneficial impact on public health. However, many strains of pathogens became antibiotic resistant, and some even became resistant to chemotherapeutic agents. This phenomenon is commonly termed multidrug resistance (or MDR).

An even more serious threat may be the emergence of gram-negative pathogens that are resistant to essentially all of the available agents. The abuse of antibiotics used for human therapy, as well as for farm animals and even for fish in aquaculture, resulted in the selection of pathogenic bacteria resistant to multiple drugs as well as in the presence of residual antibiotics in food products. For example, unique strains of the bacteria Vibrio parahaemolyticus and related Vibrio species caused acute hepatopancreatic necrosis disease (AHPND) in farmed populations of marine shrimp (Kumar et al., Review in Aquaculture, 12: 1867-1880, 2020).

The presence of bacteria, in particular the aforementioned Vibrio bacteria, in plants and marine species has led to increased numbers of infectious disorders, such as vibriosis, in subjects that consumed undercooked or raw shellfish and agriculture produce. These bacteria also lead to significant losses in aquaculture, such as shrimp farms, and agriculture.

There is an unmet need for alternative antibacterial platforms, species and treatments.

### SUMMARY

In some embodiments, there is provided a genetically engineered non-pathogenic bacterium comprising a gene cluster encoding an antibacterial protein delivery platform for rendering antibacterial properties to the non-pathogenic bacterium, wherein the gene cluster is operably linked to an inducible positive regulation system and further comprising at least one effector and immunity pair, where the delivery of the effector within the pair exerts said antibacterial activity.

The non-pathogenic bacterium disclosed herein, has been transformed into an antibacterial tool using an engineered antibacterial Type VI secretion system (T6SS), as a result said non-pathogenic bacterium possesses antibacterial activity which can be controlled using various external signals. Accordingly, the genetically engineered non-pathogenic bacteria disclosed herein are functionally silent until they reach an inducing environment, e.g., an external molecule secreted by a pathogen, which induces expression of antibacterial toxin(s), namely, the expression of at least one effector-immunity pair (also termed herein ‘effector and immunity pair’), and expression of the gene cluster encoding an antibacterial protein delivery platform. The T6SS gene cluster from which the engineered T6SS exemplified herein was derived typically utilizes two major positive regulators for exerting its antibacterial activity: vp1407 and vp1391 (FIG. 1A). In the absence of either VP1407 or VP1391, the T6SS protein delivery platform is not constructed and therefore cannot mediate antibacterial activity. Surprisingly, complemented expression of only one native regulator—vp1407 (which was removed from the engineered T6SS cluster), alone or within the operon vp1409-1407, was sufficient to regain antibacterial activity in the genetically engineered non-pathogenic bacteria. Unexpectedly, vp1407 was found to have the capability of operating or shutting off the protein delivery platform (on/off switch). As it is found upstream in the T6SS gene cluster regulation cascade, VP1407 can activate, either directly or indirectly, expression of a plurality of operons in the cluster, including the operon comprising vp1391 (FIGS. 1A-1B). Therefore, in its absence none of the cluster operons is expressed. In contrast, the second positive regulator, VP1391, controls only one of the operons comprising structural T6SS apparatus genes, and therefore cannot be used as an on/off switch of the entire T6SS gene cluster. Moreover, the use of VP1391 as a positive regulator could be energy-wasting, due to constitutive expression of several T6SS gene cluster operons.

Advantageously, the engineered non-pathogenic bacterium disclosed herein may be loaded with any desired arsenals of antibacterial effectors, conferring any required target range. Moreover, since the engineered non-pathogenic bacterium relies on brute force to deliver toxic effector proteins into other cells, and since it can deliver a diverse arsenal of effectors that target multiple essential cell components, the development of resistance/immunity against a T6SS-mediated attack as disclosed herein, is not expected.

The engineered non-pathogenic bacterial platform disclosed herein is inducible and responsive to its environment. Furthermore, it is modular, allowing rapid customization. In addition, it allows manipulation of the delivered effector payload in order to control toxicity range. Moreover, it can be installed in any non-pathogenic bacterium.

The terms “toxin”, “effector” (alone or in the context of the term ‘effector-immunity pair’) and “toxic effector protein” as used herein are exchangeable, referring to the toxin that provides the induced antibacterial activity exerted by the genetically engineered non-pathogenic bacteria.

Another advantage attributed to the design of the engineered non-pathogenic bacterium is that it includes safety mechanisms which prevent acquisition of undesired activity and also prohibit transformation of a functional T6SS from the non-pathogenic bacterium, into a pathogenic one. An exemplary safety mechanism disclosed herein is achieved by the separation of the T6SS cluster at least from the inducible positive regulation system, such that each of the aforementioned components is located on a different genomic locus within the non-pathogenic bacterium.

Other safety mechanisms include, but are not limited to, deletion of tfoX, a conserved regulatory component of the DNA uptake machinery, in order to prevent V. natriegens from acquiring external virulence traits from its victims (and become virulent), via gene transfer.

The pathogen from which the T6SS platform exemplified herein was derived, is Vibrio parahaemolyticus which was found to grow preferentially on high salt media. T6SS1 in Vibrio parahaemolyticus functions optimally at 30° C. Advantageously, in the non-pathogenic bacteria disclosed herein, the aforementioned engineered T6SS platform is functional at a very wide range of temperature, namely, from about 20° C. to about 37° C., with an optimum activity at 28-30° C., and weaker activity at 20° C., presumably, due to slower growth rate of the bacterium at this temperature. Furthermore, the non-pathogenic bacteria, Vibrio natriegens, grows well on high salt media, however, as exemplified herein (see, for example, FIG. 3C), the disclosed engineered non-pathogenic bacteria platform is active throughout a wide range of salinity. Moreover, Vibrio natriegens is devoid of known virulence factors, and is devoid of endogenous T6SS. In addition, Vibrio natriegens and the marine pathogen, V. parahaemolyticus, can co-colonize the gut of a marine animal. The aforementioned characteristics beneficially render the engineered non-pathogenic bacteria hosting the T6SS platform suitable to safely treat and prevent aquaculture bacterial infections. In fact, the T6SS-engineered non-pathogenic bacteria disclosed herein may be highly useful for treating bacterial-mediated infections in shrimp, fish, and oyster farms. Moreover, treating bacterial-mediated infections as disclosed herein can be utilized to ensure the safety of consuming raw animal-based food products (e.g. fish and shellfish). Pre-treatment of such food products may prevent, or significantly reduce, the toxic effects of pathogenic bacteria residing within the raw food, on the human body following consumption of the infected food product.

It is to be understood that Vibrio natriegens is used herein merely for exemplifying the platform of engineered non-pathogenic bacteria. However, this platform can be applied using any bacterium and antibacterial system, based on the teaching and guidance provided herein.

According to some embodiment, there is provided a genetically engineered non-pathogenic bacterium having antibacterial activity, comprising a gene cluster encoding an antibacterial protein delivery platform for producing antibacterial activity, operably linked to a signal-inducible positive regulation system, and at least one effector-immunity pair, delivery of which exerts the antibacterial activity wherein the gene cluster and the positive regulation system are localized in different genomic loci within the non-pathogenic bacterium.

According to some embodiments, the positive regulation system comprises at least one activator associated with at least one promoter, configured to activate the antibacterial protein delivery platform to produce the antibacterial activity, wherein the at least one activator is a signal receptor/regulator configured to associate with at least one signal activated promoter, upon sensing an activating signal.

According to some embodiments, the positive regulation system further comprises a plurality of regulators, each associated with at least one promotor.

According to some embodiments, the positive regulation system further comprises at least one gene cluster activator, and wherein the at least one signal activated promoter induces expression of the at least one gene cluster activator, which upon association with at least one gene cluster promoter within the gene cluster, activates the antibacterial protein delivery platform to produce antibacterial activity.

According to some embodiments, the gene cluster, the signal receptor/regulator and the at least one effector-immunity pair are localized in different genomic loci within the non-pathogenic bacterium.

According to some embodiments, the gene cluster, the signal receptor/regulator, the at least one gene cluster activator and the at least one effector-immunity pair are localized in different genomic loci within the non-pathogenic bacterium.

According to some embodiments, the activating signal is an external signal exerted by a pathogen.

According to some embodiments, the at least one effector-immunity pair and the gene cluster are derived from the same bacterial strain.

According to some embodiments, the at least one effector-immunity pair and the gene cluster are derived from different bacterial strains.

According to some embodiments, the gene cluster is encoding an antibacterial Type VI secretion system (T6SS) devoid of a T6SS positive regulation system and T6SS effectors-immunity pairs.

As used herein a gene cluster encoding an antibacterial Type VI secretion system (T6SS) devoid of a T6SS positive regulation system and functional T6SS effectors-immunity pairs is also termed herein an effectorless T6SS, T6SS^{effectorless}, effectorless platform and the like.

According to some embodiments, the gene cluster is derived from a pathogen.

According to some embodiments, said bacterial strain from which the genetically engineered non-pathogenic bacterium is derived, is devoid of an endogenous T6SS.

According to some embodiments, the bacterial strain from which the genetically engineered non-pathogenic bacterium is derived, is a marine bacterium.

According to some embodiments, the gene cluster is located on a plasmid.

According to some embodiments, the at least one effector-immunity pair is located on a chromosome.

According to some embodiments, the gene cluster and the at least one effector-immunity pair are located on the same or different chromosomes in the non-pathogenic bacterium.

According to some embodiments, there is provided a composition comprising the non-pathogenic bacterium disclosed herein and a carrier.

According to some embodiments, the composition is in a dry, lyophilized, form.

According to some embodiments, the composition is in a bacteria culture form.

According to some embodiments, there is provided a method of treating pathogen-infected organisms, the method comprising:
(a) contacting the infected organisms with a composition comprising the non-pathogenic bacteria of claim 1, wherein the at least one effector-immunity pair is capable of reducing or eliminating activity or proliferation of the pathogens; and
(b) activating the positive regulation system, thereby inducing the non-pathogenic bacteria to exert antibacterial activity.

According to some embodiments, the infected organisms are marine organisms in an aqueous environment.

According to some embodiments, said contacting comprises adding the composition to the aqueous environment.

According to some embodiments, the gene cluster is encoding an antibacterial Type VI secretion system (T6SS).

According to some embodiments, the T6SS is derived from marine pathogen.

According to some embodiments, said non-pathogenic bacterium is devoid of an endogenous T6SS.

According to some embodiments, said non-pathogenic bacterium is a marine bacterium.

According to some embodiments, the gene cluster encoding the antibacterial protein delivery platform is derived from a pathogenic bacterium.

According to some embodiments, the gene cluster and the at least one effector-immunity pair are located on the same or different chromosomes in the non-pathogenic bacterium.

According to some embodiments, the external signal is a bacterial pathogen, or a molecule derived therefrom, present within the aqueous environment.

According to some embodiments, the external signal is a compound added to the aqueous environment, in order to activate the antibacterial protein delivery platform.

According to some embodiments, there is provided a kit comprising at least one receptacle containing the composition disclosed herein, for treating infected organisms and environments, and further comprising instructions for use.

According to some embodiments, the kit further comprising a positive control configured to verify that the composition in the at least one receptacle, is active.

According to some embodiments, the positive control comprises a sample of an activating signal molecule and a detector of secreted platform components.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1A exhibits the regulatory cascade activating Vibrio parahaemolyticus RIMD 2210633 T6SS1 gene cluster: TfoY is a master regulator required for expression of the vp1409-7 operon. The expressed regulator, VP1407, then activates expression of additional operons (vp1392-1, vp1400-6, vp1410-20, and vp1386-7), including its own operon, vp1409-7. In turn, a second regulator that is induced by VP1407, namely, VP1391, activates expression of the final structural T6SS1 operon, vp1393-9, and the vp1388-90 operon, thereby completing the expression of the cluster and resulting in construction of a functional T6SS. Dashed arrows denote transcriptional activation.

FIG. 1B exhibits the regulatory cascade activating the effectorless (devoid of vp1388-90 and vp1407 and further carrying inactivating catalytic mutations in the nuclease toxin domain of VP1415) engineered T6SS platform in Vibrio natriegens: an external signal activates a regulator that induces expression of the T6SS activator VP1407. VP1407 then induces expression of several T6SS cluster operons (vp1392-1, vp1400-6, vp1410-20, and vp1386-7), including the operon it originated from in the natural T6SS1 encoded by Vibrio parahaemolyticus RIMD 2210633, which here includes only vp1409-8. In turn, a second T6SS cluster-encoded regulator that is induced by VP1407, namely, VP1391, activates expression of the final structural T6SS1 operon, vp1393-9, thereby completing the expression of the cluster and resulting in construction of a functional T6SS. Dashed line arrows denote transcriptional activation of operons.

FIG. 1C left panel illustrates pathogen-targeting lethal activity of an engineered non-pathogenic bacterium containing engineered T6SS gene cluster (namely, T6SS containing exogenous effector and immunity pair(s) and devoid of T6SS native effectors and positive regulation system), induced by an activating signal (triangle; optionally the signal is an external signal produced by the pathogen), through the positive regulation system: upon binding a signaling molecule, the T6SS signal receptor/regulator associates with an external signal-activated promoter which in turn activates the T6SS activator, that induces the activity of the T6SS cluster and activates the engineered T6SS platform to deliver antibacterial effector(s) into the pathogen; the right panel illustrates an engineered non-pathogen bacterium containing T6SS gene cluster, in the absence of an activating signal: the signal receptor/regulator does not associate with the external signal-activated promoter, thus T6SS delivery platform is inactive.

FIG. 1D exhibits expression (in cells) and secretion (to the culture media) of VgrG1 from V. parahaemolyticus RIMD 2210633 derivative PORI (Vpara; T6SS1⁺) and its T6SS1⁻ mutant (Vpara/Δhcp1), and from V. natriegens (Vnat) carrying the indicated plasmids. Arrows denote bands corresponding to VgrG1, asterisks denote non-specific bands detected in Vnat samples.

FIG. 1E exhibits viability counts of V. natriegens prey before (0 h; upward triangle) and four hours after (4 h; circle) co-incubation with the indicated attackers: V. parahaemolyticus RIMD 2210633 derivative PORI (Vpara; T6SS1⁺) and its T6SS1⁻ mutant (Vpara/Δhcp1), and from V. natriegens (Vnat) carrying the indicated plasmids, as described in FIG. 1D, on media containing 3% NaCl at 30° C. Data shown as mean±SD, statistical significance between samples at the 4 h timepoint by an unpaired, two-tailed Student's t-test are denoted above, where significant difference was considered as P<0.05 (DL, assay detection limit).

FIG. 2A exhibits viability counts of V. natriegens prey before (0 h, downward triangle) and four hours after (4 h, circle) co-incubation with wild-type V. natriegens attackers harboring the indicated plasmids, on media containing 3% NaCl at 30° C. Data are shown as mean±SD, and statistical significance between samples at the 4 h timepoint by an unpaired, two-tailed Student's t-test are denoted above where significant difference was considered as P<0.05.

FIG. 2B exhibits expression (cells) and secretion (media) of VgrG1 from V. natriegens containing an arabinose-inducible vp1407 (Vnat^{Reg}) or vp1409-7 in the chromosomal dns locus and harboring the indicated plasmids, in samples grown in media containing 3% NaCl and either supplemented (+) or not (−) with 0.1% arabinose (Ara) at 30° C. Loading control (LC) is shown for total protein lysates.

FIG. 2C illustrates an engineered Vnat^{Reg} bacteria containing a plasmid-borne, inducible VpT6SS1 (pT6SS1^{Ind}), where in the presence of arabinose (circles), VP1407 is expressed from the chromosome and the plasmid-borne T6SS is induced, resulting in T6SS-mediated intoxication of adjacent prey bacteria while intoxication is not taking place in the absence of arabinose.

FIG. 2D exhibits viability counts of V. natriegens prey before (0 h; downward triangle) and after (4 h) co-incubation with the indicated V. natriegens attackers carrying the indicated plasmids on solid media plates in the absence or presence of arabinose (upward triangle and circle, respectively), as described in FIG. 3D. Data are shown as mean±SD, and statistical significance between samples at the 4 h timepoint by an unpaired, two-tailed Student's t-test are denoted above where significant difference was considered as P<0.05.

FIG. 3A exhibits viability counts of V. natriegens prey before (0 h; downward triangle) incubation, four and 24 hours after (4/24 h) co-incubation with the indicated V. natriegens attackers harboring the indicated plasmids, on media containing 3% NaCl and 0.1% arabinose at 20° C., 28° C., 30° C. and 37° C. (upward triangle, circle, diamond and square, respectively). Data are shown as mean±SD, and statistical significance between samples at the 4 h timepoint by an unpaired, two-tailed Student's t-test are denoted above (in the same order as the 4/24 h shape legends are shown) where significant difference was considered as P<0.05.

FIG. 3B exhibits V. natriegens—wild-type (WT) and the Vnat^{Reg} derivative, growth at different temperatures in media containing 3% NaCl, as measured by OD600 readings. Data are shown as the mean±SD of 16 datapoints collected as technical quadruplicates in 4 independent experiments.

FIG. 3C exhibits viability counts of V. natriegens prey before (0 h; downward triangle) and 4 hours after co-incubation with the indicated V. natriegens attackers harboring the indicated plasmids, on media containing 1-5% (w/v) NaCl and 0.1% arabinose at 28° C. (square, upward triangle, circle, downward triangle and diamond, respectively). Data are shown as means (n=3), and statistical significance between samples at the 4 h timepoint by an unpaired, two-tailed Student's t-test are denoted above (in the same order as the shape legends are shown) where significant difference was considered as P<0.05.

FIG. 3D exhibits viability counts of prey bacteria V. parahaemolyticus 12-297/B Δhcp1, before (0 h; downward triangle) and four hours after (4 h; circle) co-incubation with the indicated V. natriegens attackers harboring the indicated plasmids, on media containing 3% NaCl and 0.1% arabinose at 28° C. Data shown as mean±SD. Statistical significance between samples at the 4 h timepoint by an unpaired, two-tailed Student's t-test are denoted above. Significant difference was considered as P<0.05.

FIG. 3E exhibits viability counts of prey bacteria V. parahaemolyticus 04.2548 Δhcp1, before (0 h; downward triangle) and four hours after (4 h; circle) co-incubation with the indicated V. natriegens attackers harboring the indicated plasmids, on media containing 3% NaCl and 0.1% arabinose at 28° C. Data shown as mean±SD. Statistical significance between samples at the 4 h timepoint by an unpaired, two-tailed Student's t-test are denoted above. Significant difference was considered as P<0.05.

FIG. 3F exhibits viability counts of prey bacteria V. vulnificus CMCP6, before (0 h; downward triangle) and four hours after (4 h; circle) co-incubation with the indicated V. natriegens attackers harboring the indicated plasmids, on media containing 3% NaCl and 0.1% arabinose at 28° C. Data shown as mean±SD. Statistical significance between samples at the 4 h timepoint by an unpaired, two-tailed Student's t-test are denoted above. Significant difference was considered as P<0.05.

FIG. 3G exhibits viability counts of prey bacteria A. jandaei DSM 7311 ΔtssB, before (0 h; downward triangle) and four hours after (4 h; circle) co-incubation with the indicated V. natriegens attackers harboring the indicated plasmids, on media containing 3% NaCl and 0.1% arabinose at 28° C. Data shown as mean±SD. Statistical significance between samples at the 4 h timepoint by an unpaired, two-tailed Student's t-test are denoted above. Significant difference was considered as P<0.05.

FIG. 4A exhibits viability counts of V. natriegens prey before (0 h; upward triangle) and after (4 h; circle) co-incubation with Vnat^{Reg} attackers harboring the indicated plasmids, on media containing 3% NaCl and 0.1% arabinose at 28° C. Data are shown as the mean±SD. Statistical significance between samples at the 4 h timepoint by an unpaired, two-tailed Student's t-test are denoted above. A significant difference was considered as P<0.05.

FIG. 4B exhibits expression (cells) and secretion (media) of VgrG1 from Vnat^{Reg} harboring the indicated plasmids. Samples were grown in media containing 3% NaCl and 0.1% arabinose at 28° C. Loading control (LC) is shown for total protein lysates. An arrow denotes bands corresponding to VgrG1. An asterisk denotes non-specific bands.

FIGS. 5A-5E exhibit viability counts of V. vulnificus (5A), Aeromonas jandaei ΔtssB (5B), Salmonella enterica (5C), E. coli (5D) and V. natriegens (5E) prey survival when competed alone (single prey) against the attacker at a 4:1 (attacker:prey) ratio before (0 h) and four hours after (4 h) co-incubation with the indicated V. natriegens attackers (Vnat^{Reg}) harboring the indicated plasmids, on media containing 3% NaCl at 28° C. Statistical significance between samples at the 4 h timepoint by an unpaired, two-tailed Student's t-test are denoted above. Significant difference was considered as P<0.05. Effector plasmids encode PoNe/i together with VgrG1b from V. parahaemolyticus 12-297/B (pPoNe/i^{Vp 12-297/B}), Tme/i1 from V. parahaemolyticus BB22OP (pTme/i1^{Vp BB22OP}) VPA1263-Vti2 from V. parahaemolyticus RIMD 2210633 (pVPA1263-Vti2^{Vp RIMD}) and Va02265-0 from V. alginolyticus 12G01 (pVa02265-0^{Va 12G01}).

FIGS. 5F-5J exhibit viability counts of V. vulnificus (5F), Aeromonas jandaei ΔtssB (5G), Salmonella enterica (5H), E. coli (5I) and V. natriegens (5J) prey following competition in which all prey were mixed together with the attacker at a 10:1:1:1:1:1 (attacker:prey) ratio before (0 h) and four hours after (4 h) co-incubation with the indicated V. natriegens attackers (Vnat^{Reg}) harboring the indicated plasmids (as described in above for FIGS. 5A-5E), on media containing 3% NaCl at 28° C. Statistical significance between samples at the 4 h timepoint by an unpaired, two-tailed Student's t-test are denoted above. Significant difference was considered as P<0.05.

FIGS. 6A-6C exhibit viability counts of V. parahaemolyticus RIMD 2210633 Δhcp1 (6A), V. alginolyticus 12G01 Δhcp1/Δhcp2 (6B), and V. natriegens (6C) prey bacteria before (0 h; downward triangle) and after (4 h; circles) co-incubation with the indicated V. natriegens attackers harboring the indicated plasmids (plasmids are as described in FIGS. 5A-5B; pVPA1263-Vti2+Va02265-0 encodes both VPA1263-Vti2 from V. parahaemolyticus RIMD 2210633 and Va02265-0 from V. alginolyticus 12G01), on media containing 3% NaCl and 0.1% arabinose at 28° C. Data shown as mean±SD. Statistical significance between samples at the 4 h timepoint by an unpaired, two-tailed Student's t-test are denoted above. Significant difference was considered as P<0.05.

FIG. 6D illustrates the competitions shown in FIG. 6A-6B: effector and immunity modules expressed by Vnat^{Reg} carrying pT6SS^{Effectorless} (middle bacteria; effector and immunity modules denoted as a circle and triangle, respectively) are colored white/grey to match the bacterium from which they were derived.

FIGS. 7A-7D demonstrate representative fluorescence microscope images of Artemia nauplii colonization by V. parahaemolyticus strain T9109 Δhcp1 derivative expressing GFP (Vpara; FIGS. 7A-7C) and V. natriegens ATCC 14048 constitutively expressing RFP (Vnat; FIGS. 7A, 7B and 7D), 24 hours after immersion challenge.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

In some embodiments, there is provided a non-pathogenic bacterium comprising a gene cluster encoding an antibacterial protein delivery platform for rendering antibacterial properties to the bacterium, wherein the gene cluster is operably linked to a signal-inducible positive regulation system.

The terms “non-pathogenic bacterium” and “non-pathogenic bacteria” as used herein are interchangeable and refer to bacteria that are not capable of causing disease or harmful responses in a host. In some embodiments, non-pathogenic bacteria are Gram-negative bacteria. Examples of non-pathogenic bacteria include, but are not limited to Vibrio, Bacteroides, Escherichia, Pseudomonas e.g., Vibrio natreigens, Bacteroides fragilis, Bacteroides subtilis, Bacteroides thetaiotaomicron, Escherichia coli, Pseudomonas putida and Pseudomonas fluorecences.

As used herein, the term “gene” or “gene sequence” is meant to refer to a nucleic acid sequence encoding any of the genetic components disclosed herein, such as, the antibacterial protein delivery platform, the components of the positive regulation system, including, activators, regulators and effectors, as well as others. The nucleic acid sequence may comprise the entire gene sequence, or a partial gene sequence encoding a functional molecule, with or without the corresponding promoter region. The nucleic acid sequence may be a natural sequence or a synthetic sequence. The nucleic acid sequence may comprise a native or wild-type sequence or may comprise a modified sequence having one or more insertions, deletions, substitutions, or other modifications, for example, the nucleic acid sequence may be codon-optimized.

As used herein, a “gene cluster” or “operon” refer to two or more genes that are required to produce an anti-bacterial function. In addition to encoding a set of genes capable of producing molecule, such as, toxin/effector, the gene cluster or operon may also comprise additional transcription and translation elements, e.g., a ribosome binding site, promoter, terminator and the like.

In some embodiments, the signal-inducible positive regulation system is inducibly-expressed by external signals. In some embodiments, the signal-inducible positive regulation system is inducibly-expressed by external signals from a genomic location.

Each gene or gene cluster may be operably linked to an inducible promoter, e.g., a pathogenic signal-activated promoter, which activates the signal receptor/regulator. An “inducible promoter” refers to a regulatory region that is operably linked to one or more genes, wherein expression of the gene(s) is increased in the presence of an inducer of said regulatory region.

Thus, in some embodiments, the positive regulation system is operably linked to the at least one effector-immunity pair. Accordingly, in some embodiments, the positive regulation system is operably linked to the antibacterial protein delivery platform and to at least one effector-immunity pair.

The term “Operably-linked” refers to the association of nucleic acid sequences so that the function of one is affected by the other. For example, a promoter is operably-linked with a coding sequence when it is capable of affecting the expression of that coding sequence. The operable linkage may be direct or indirect, namely, the promoter e.g. of the effector, may be directly activated by the regulation system, or it may be (indirectly) activated by a different component that has been activated by the regulation system.

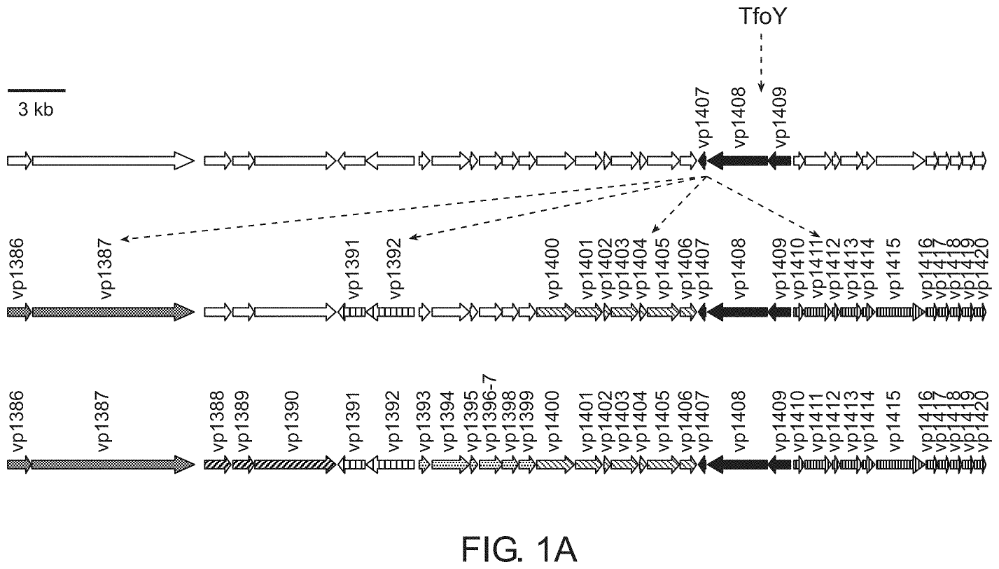

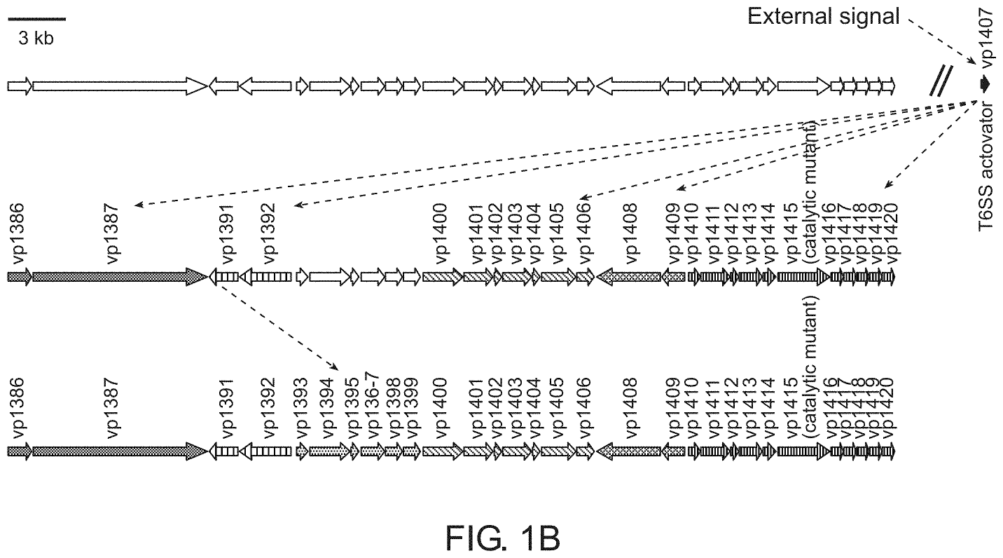

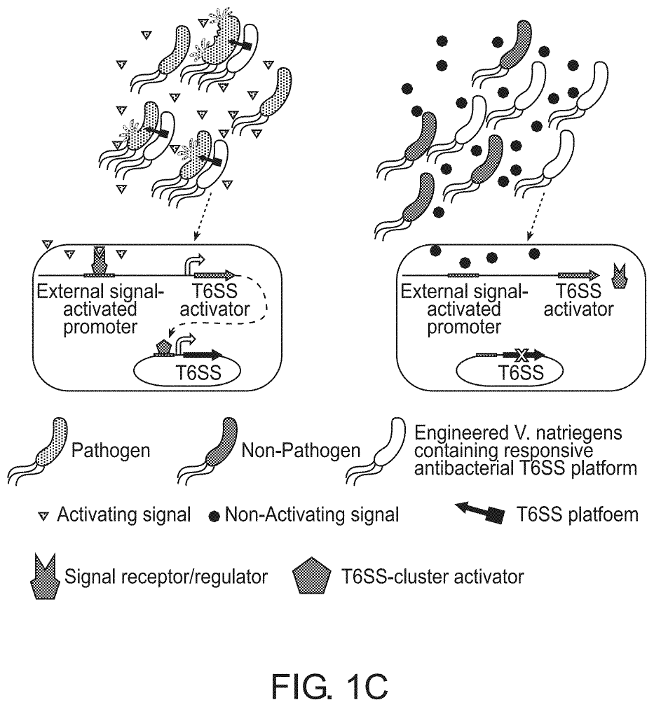

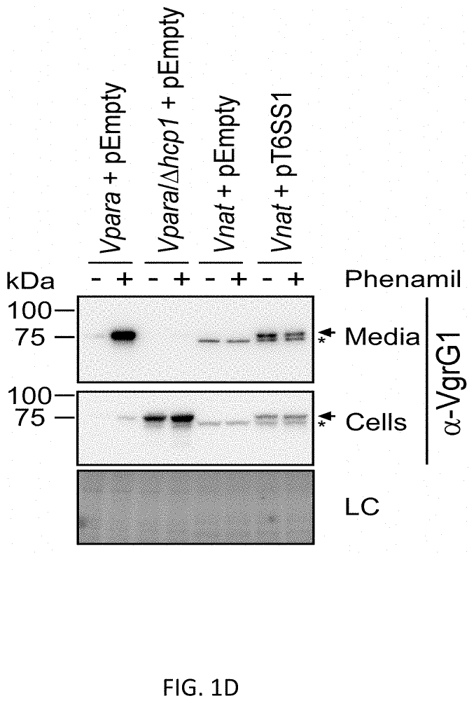

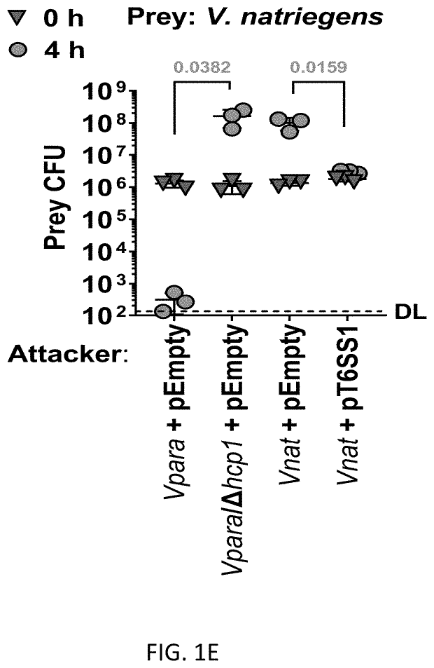

As used herein, “signal” refers to settings or circumstances under which the positive regulator is induced, wherein the positive regulator is controlled by a promoter that is responsive to an activator/repressor, the latter is capable of sensing a signaling molecule (e.g. external activating signal) thereby activating the gene cluster (see, for example, FIG. 1C). The signal may be an external signal, namely, a signal which is meant to refer to the environmental conditions external to the engineered bacteria, which may be endogenous or native to the environment in which it is active, or required to be active, for example, an infected human body or organ, an infected plant, plantation, and an infected aquaculture farm. In some embodiments, the exogenous environmental conditions are specific to aquaculture farming.

In some embodiments, the gene cluster encoding the antibacterial protein delivery platform is devoid of its innate activation components and/or effector and immunity-containing operon or the latter are present in an inactivated form. Accordingly, the engineered non-pathogenic bacterium disclosed herein may be loaded with desired arsenals of antibacterial toxin (effectors) and their cognate immunity proteins.

Thus, the non-pathogenic antibacterial cell disclosed herein, engineered with an inducible antibacterial protein delivery platform is controlled by an extracellular signal to kill competing bacterial pathogens. Moreover, the repertoire of toxic antibacterial activities of this platform can be controlled by altering the repertoire of delivered toxins (also termed herein “effector repertoire”). Hence, the platform disclosed herein may be used for inducing and/or transporting engineered effectors having desired activity/ies and target range. Hence, the platform disclosed herein may be used for generating custom-made antibacterial tools.

In some embodiments, the at least one effector-immunity pair and the non-pathogenic bacteria are derived from the same bacterial strain.

In some embodiments, the at least one effector-immunity pair and the non-pathogenic bacteria are derived from different bacterial strain.

In some embodiments, the at least one effector-immunity pair and the antibacterial protein delivery platform are derived from the same pathogen.

In some embodiments, the at least one effector-immunity pair and the antibacterial protein delivery platform are derived from different pathogenic strains.

The genetically engineered non-pathogenic bacterium disclosed herein is designed with various safety mechanisms, configured to avoid uncontrolled activation of the protein delivery system. One of the safety mechanisms includes using a modified protein delivery system devoid of its positive regulation and/or effector-immunity pairs, and hence is inactive. Its activity is achieved by external regulators, located outside and away from the gene cluster encoding the protein delivery platform. To this end, at least the positive regulation system of the platform, or components thereof (such as, one or more promoter, activator, regulator) are located in a genomic locus different from the location of the platform, while being operably linked.

In some embodiments, the positive regulation system, the at least one effector-immunity pair, and the antibacterial protein delivery platform are located at different genomic loci of the non-pathogenic bacterium.

In some embodiments, the positive regulation system is located on a plasmid in the non-pathogenic bacterium. In some embodiments, the gene cluster encoding the antibacterial protein delivery platform is located on a plasmid in the non-pathogenic bacterium. In some embodiments, the at least one effector-immunity pair is located on a plasmid in the non-pathogenic bacterium.

In some embodiments, the plasmid in the engineered bacteria is a stably maintained plasmid capable of producing an antibacterial function upon being activated. In some embodiments, the plasmid is a low-copy plasmid. In some embodiments, the low-copy plasmid may be useful for increasing stability of expression. In some embodiments, the low-copy plasmid may be useful for decreasing leaky expression under non-inducing conditions. In some embodiments, the plasmid is a high-copy plasmid. In some embodiments, the high-copy plasmid may be useful for increasing the gene cassette expression.

In some embodiments, at least one of the positive regulation system, the gene cluster encoding the antibacterial protein delivery platform and the at least one effector-immunity pair is located on a chromosome in the non-pathogenic bacterium. In some embodiments, a plurality of the at least one of the positive regulation system, the gene cluster encoding the antibacterial protein delivery platform and the at least one effector-immunity pair is located on a chromosome in the non-pathogenic bacterium, preferably on different loci of the same chromosome, or on different chromosomes.

Inserting the gene cluster and/or the positive regulation system and/or the at least one effector-immunity pair into the chromosome of the non-pathogenic bacteria is intended for obtaining stable expression thereof and hence prevent their loss over time. Also, inserting the gene cassette into the chromosome may reduce the chance of other bacteria acquiring the gene-cluster when located in a plasmid. However, the latter is of less concern since the platform encoded by the gene cluster will not be active in other bacteria because it is not located on the same location of the regulation system and/or the at least one effector-immunity pair, within the genome of the non-pathogenic bacteria.

As used herein, “stable expression” is used to refer to a bacterial host cell carrying non-native genetic material, e.g., the gene cluster encoding the antibacterial protein delivery platform, which is incorporated into the host genome or propagated on a self-replicating extra-chromosomal plasmid, such that the non-native genetic material is retained, expressed, and/or propagated. The stable bacterium is capable of survival and/or growth in vitro, e.g., in medium, and/or in vivo, e.g., in an organism.

T6SS

In some embodiments, the antibacterial protein delivery platform is Type VI secretion system (T6SS). Accordingly, in some embodiments, the gene cluster encoding the antibacterial protein delivery platform is T6SS gene cluster.

As used herein, “T6SS gene cluster” and “T6SS cluster” are used interchangeably to refer to a set of genetically engineered T6SS system capable of producing antibacterial protein delivery platform. The main genes of the genetically engineered T6SS system disclosed herein are illustrated in FIG. 1B and SEQ ID NO: 1. In some embodiments, the genetically engineered T6SS gene cluster is devoid of its innate activation components and/or effector and immunity-containing operon. In some embodiments, at least one of the innate activation components and effector-immunity pair(s) of the genetically engineered T6SS are inactive.

In some embodiments, the genetically engineered T6SS gene cluster is devoid of vp1407.

Thus, the terms “T655 gene cluster” and “T655 cluster” as used herein are interchangeable with the terms “engineered T6SS gene cluster” and “engineered T6SS cluster”.

The term “devoid” with respect to particular activity and/or sequence (e.g. gene) refers to either removal/deletion or inactivation of said sequence.

Type VI secretion system (T6SS) is a multi-protein machine that is used by many Gram-negative bacteria to deliver toxins, called effectors or toxic effectors, directly into adjacent cells in a contact-dependent manner. Although a few T6SSs were shown to mediate anti-eukaryotic activities, the vast majority of T6SSs mediate antibacterial toxicity, and therefore they play a major role in inter-bacterial competition. The toxic effectors are encoded within a gene locus that also encodes a cognate immunity protein, and sometimes a chaperone, which together form effector-immunity pair.

In some embodiments, the modified T6SS gene cluster is illustrated in FIG. 1B and its nucleotide sequence is provided as SEQ ID NO: 1. However, a modified T6SS gene cluster as used herein may include a variety of sequences, capable of safely exerting delivery of toxic effector(s), when activated.

Unmodified bacteria that are capable of producing native T6SS system include, but are not limited to, Xanthomonas, euvesicatoria, Pantoea agglomerans, Pseudomonas putida, V. parahaemolyticus, Vibrio cholerae, Acinetobacter baumannii, Salmonella enterica, Pseudomonas aeruginosa, Escherichia coli and Burkholderia pseudomallei.

Thus, in some embodiments, the modified T6SS system corresponds to a T6SS system derived from Xanthomonas, euvesicatoria, Pantoea agglomerans, Pseudomonas putida, V. parahaemolyticus, Vibrio cholerae, Acinetobacter baumannii, Salmonella enterica, Pseudomonas aeruginosa, Escherichia coli or Burkholderia pseudomallei. Each possibility is a separate embodiment of the present invention.

In some embodiments, the modified T6SS system corresponds to a T6SS system derived from V. parahaemolyticus. In some embodiments, the modified T6SS system corresponds to a T6SS system derived from Vibrio cholerae. In some embodiments, the modified T6SS system corresponds to a T6SS system derived from Acinetobacter baumannii. In some embodiments, the modified T6SS system corresponds to a T6SS system derived from Salmonella enterica. In some embodiments, the modified T6SS system corresponds to a T6SS system derived from Pseudomonas aeruginosa. In some embodiments, the modified T6SS system corresponds to a T6SS system derived from Escherichia coli. In some embodiments, the modified T6SS system corresponds to a T6SS system derived from Burkholderia pseudomallei.

The main genes of unmodified T6SS system derived from V. parahaemolyticus are illustrated in FIG. 1A. Native antibacterial Type VI secretion system platforms (SEQ ID NO: 2) include innate positive regulation system, which is adapted to activate the platform which delivers the antibacterial activity and innate effector-immunity pair(s), which exerts the antibacterial activity, once the platform is activated, by delivering into neighboring cells toxins (effectors). The T6SS cluster-borne antibacterial effector and immunity-containing operons of the T6SS disclosed herein are vp1388-90 and the genes vp1415-6 (FIG. 1A).

In some embodiments, the engineered T6SS gene cluster is derived from T6SS gene cluster of a marine pathogen. In some embodiments, the marine pathogen is V. parahaemolyticus.

In some embodiments, the non-pathogenic bacterium is a marine bacterium. In some embodiments, the non-pathogenic bacterium lacks an endogenous T6SS. In some embodiments, the non-pathogenic bacterium is Vibrio natriegens.

In some embodiments, the non-pathogenic bacterium is a plant-residing bacterium. In some embodiments, the non-pathogenic bacterium is Pseudomonas putida or Pseudomonas fuorescens. Each possibility is a separate embodiment of the present invention.

In some embodiments, the non-pathogenic bacterium is a terrestrial animal-residing bacterium. In some embodiments, the non-pathogenic bacterium is Escherichia coli.

In some embodiments, the T6SS gene cluster is present on a plasmid and is operably linked to the positive regulation system.

In some embodiments, the T6SS gene cluster is located on a chromosome in the non-pathogenic bacterium. In some embodiments, the positive regulation system is located on a chromosome in the non-pathogenic bacterium. In some embodiments, the at least one effector-immunity pair is located on a chromosome in the non-pathogenic bacterium. In some embodiments, the T6SS gene cluster and the positive regulation system are located on the same or different chromosomes. In some embodiments, the T6SS gene cluster and the at least one effector-immunity pair are located on the same or different chromosomes. In some embodiments, the positive regulation system and the at least one effector-immunity pair are located on the same or different chromosomes. In some embodiments, the T6SS gene cluster and/or the positive regulation system and/or the at least one effector-immunity pair are located on different loci of the same chromosome. In some embodiments, the T6SS gene cluster and/or the positive regulation system and/or the at least one effector-immunity pair are located on different chromosomes.

In some embodiments, the T6SS gene cluster may be present on a plasmid and the positive regulation system may be present on a chromosome, and vice versa. In some embodiments, the T6SS gene cluster may be present on a plasmid and the at least one effector-immunity pair may be present on a chromosome, and vice versa. In some embodiments, the positive regulation system may be present on a plasmid and the at least one effector-immunity pair may be present on a chromosome, and vice versa.

In some embodiments, the positive regulation system is derived from an operon of the pathogen from which the T6SS platform was derived.

In some embodiments, the at least one effector-immunity pair is derived from the pathogen from which the T6SS platform was derived. In some embodiments, the at least one effector-immunity pair is derived from the non-pathogenic bacteria. In some embodiments, the at least one effector-immunity pair is an innate effector-immunity pair of the non-pathogenic bacteria. In some embodiments, the at least one effector-immunity pair is derived from a pathogen other than the pathogen from which the T6SS platform was derived

In some embodiments, the positive regulation system comprises at least one activator associated with at least one promoter, configured to activate the antibacterial protein delivery platform to produce the antibacterial activity, wherein the at least one activator is a signal receptor/regulator configured to associate with at least one signal activated promoter, upon sensing an activating signal.

In some embodiments, the signal receptor/regulator is derived from the pathogen from which the T6SS gene cluster has been derived. In some embodiments, the signal receptor/regulator is derived from a pathogen other than the pathogen from which the T6SS gene cluster has been derived. In some embodiments, the signal receptor/regulator is derived from a non-pathogenic bacterium. In some embodiments, the signal receptor/regulator is synthetically engineered.

In some embodiments, the at least one signal activated promoter is derived from the pathogen from which the T6SS gene cluster has been derived. In some embodiments, the at least one signal activated promoter is derived from the pathogen from which the signal receptor/regulator is derived. In some embodiments, the at least one signal activated promoter is synthetically engineered.

In some embodiments, the signal receptor/regulator is signal receptor/regulator AraC gene. In some embodiments, the signal receptor/regulator comprises SEQ ID NO: 10.

In some embodiments, the positive regulation system further comprises at least one gene cluster activator and wherein the at least one signal activated promoter induces expression of the at least one gene cluster activator, which upon association with at least one gene cluster promoter within the gene cluster, activates the antibacterial protein delivery platform to produce antibacterial activity.

In some embodiments, the at least one gene cluster activator is derived from the bacterium from which the T6SS gene cluster has been derived. In some embodiments, the at least one gene cluster activator is derived from the bacterium from which the signal receptor/regulator is derived.

In some embodiments, the at least one gene cluster activator comprises SEQ ID NO: 3. In some embodiments, the at least one gene cluster activator is vp1407.

Thus, in some embodiments, the positive regulation system is a gene cassette comprising the signal receptor/regulator AraC gene, the AraC-responsive promoter, and the gene cluster activator vp1407. In some embodiments, the positive regulation cassette comprises SEQ ID NO: 11. In some embodiments, the positive regulation cassette is consisting of SEQ ID NO: 11.

In some embodiments, the positive regulation system is a gene cassette comprising the signal receptor/regulator AraC gene, the AraC-responsive promoter, and the 3-gene operon vp1409-1407 as the gene cluster activator. In some embodiments, the positive regulation cassette comprises SEQ ID NO: 12. In some embodiments, the positive regulation cassette is consisting of SEQ ID NO: 12.

The activation of the 3-gene operon vp1409-1407 or of vp1407 alone, is adapted to be made responsive when placed under inducing regulation of various signals, such as synthetic chemicals or naturally produced molecules (e.g., quorum sensing signals or bile salts). Moreover, the antibacterial effector arsenal (or effector repertoire) that is delivered by the engineered V. natriegens platform can be manipulated to include varying repertoires of effectors with diverse activities and targets. According to some embodiments, the antibacterial effector arsenal that is delivered by the engineered V. natriegens platform disclosed herein are natural effectors, namely, effectors that are naturally delivered by another bacterium carrying a homologous T6SS. Such effectors can be transferred from bacteria that harbor a homologous T6SS cluster, or they can be synthetically engineered toxins that may provide target specificity. The effectors are configured to be encoded together with a cognate immunity gene that protects the bacterium against self- or kin-intoxication by the effector.

Compositions and Kits

In some embodiments, there is provided a composition comprising the non-pathogenic bacterium disclosed herein and an acceptable carrier and/or excipient. In some embodiments, the composition comprises the non-pathogenic bacterium in a lyophilized form.

In some embodiments, the composition is a dry powder. In some embodiments, the composition comprises the non-pathogenic bacterium in the form of bacterial culture.

In some embodiments, the composition is a pharmaceutical composition suitable for treatment of infectious diseases.

In some embodiments, the pharmaceutical composition is in the form or aerosol, suitable for use through nebulizer or inhalers for the treatment of infectious diseases via inhalation.

In some embodiments, the composition is in a liquid form. In some embodiment the composition is sprayable.

The terms “carrier” and “excipient” which may be used interchangeably refer to a component that does not abrogate the activity and properties of the engineered bacteria. This compound may be required, for example, for conferring stability to the composition, when in storage.

In some embodiments, there is provided a kit comprising the composition disclosed herein for treating pathogen-infected organisms and/or environments containing pathogen-infected organisms, and further comprising instructions for use, wherein the at least one effector-immunity pair is capable of reducing or eliminating activity or proliferation of the pathogen.

In some embodiments, the instructions for use include contacting the infected organisms and/or and environments, with the composition disclosed in the kit.

In some embodiments, the kit comprises at least one receptacle encompassing therewithin the composition.

In some embodiments, the kit comprises a plurality of receptacles, each encompassing therewithin a sample of the composition.

In some embodiments, the kit may further comprise a sample of the pathogen, as a positive control, configured to verify that the composition in the at least one receptacle, is active. As the latter may be unsafe, the kit may further comprise a sample of an activating signal molecule and a detector of secreted platform components (e.g. the T6SS structural secreted components Hcp or VgrG, or an effector) configured to verify that the composition in the at least one receptacle, is active.

In some embodiments, each of said positive control components is provide in one or more separate receptacles.

In some embodiments, the composition in the at least one receptacle is in a dry form. In some embodiments, the genetically modified non-pathogenic bacteria in the kit are in a lyophilized or dry form, wherein the kit further comprises at least one container containing liquid for reviving the lyophilized or dry form non-pathogenic bacteria.

In some embodiments, the at least one receptacle is in the form of a cartridge suitable to be incorporated within a nebulizer, and inhaler or any aerosol forming device.

Methods for using the non-pathogenic bacteria.

In some embodiments, there is provided a method for treating pathogen-infected organisms, the method comprising
a. contacting the infected organisms with a composition comprising the non-pathogenic bacteria disclosed herein, wherein the at least one effector-immunity pair is capable of reducing or eliminating activity or proliferation of the pathogen; and
b. activating the positive regulation system, thereby inducing the non-pathogenic bacteria to exert antibacterial activity.

The term “organisms” in the context of the present disclosure includes plants, animals and humans. Thus, the infected organisms may be human consuming infected plants, sea food, meat and the like. It is important to note that the present antibacterial compositions and methods may be used to eliminate or reduce the effect of pathogens which may be not pathogenic in the food product where they reside (e.g. shrimp), but may be extremely pathogenic and even lethal to human consuming the infected food product. For example, consumption of raw oysters that contain large amounts of pathogenic Vibrio vulnificus or Vibrio parahaemolyticus can result in severe vibriosis and even death in humans (Kumar et al., ibid).

In some embodiments, activating the positive regulation system includes adding the composition to an environment where the pathogen-infected organisms are located. This may include spraying the composition onto plants, plantations, aquaculture farms and public areas, wherein the activation occurs when the genetically engineered non-pathogenic bacteria contact the pathogen or molecules associated therewith (e.g. secreted by the pathogen).

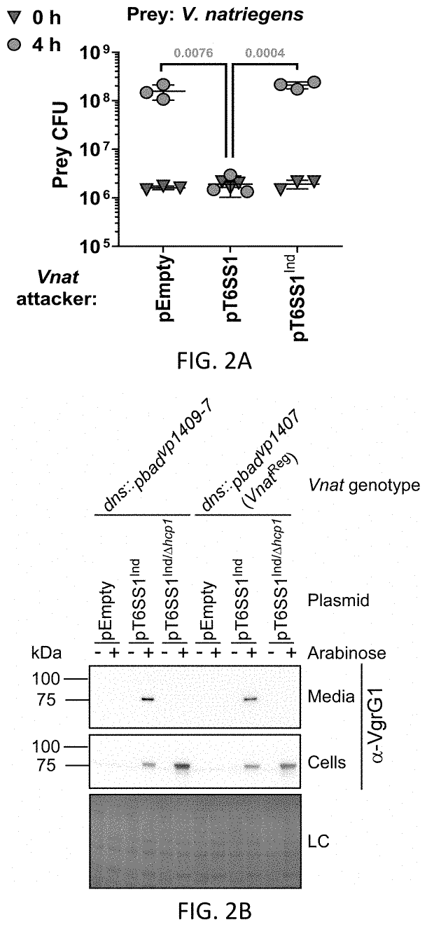

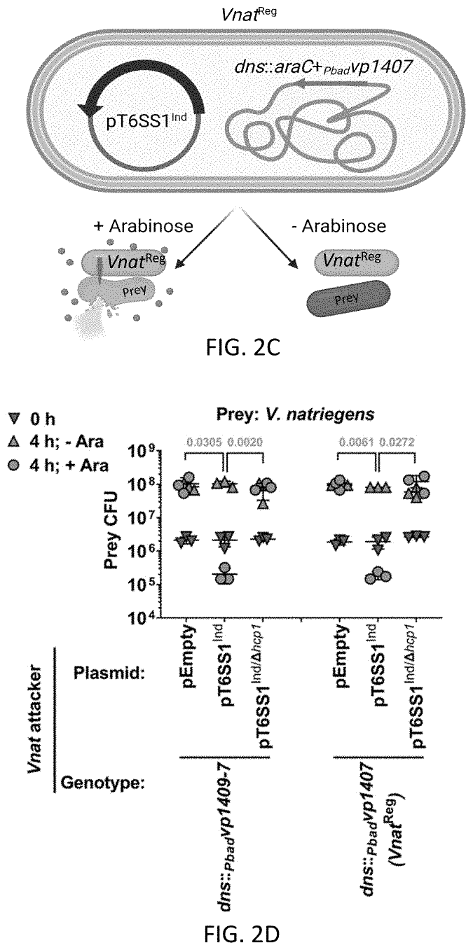

In some embodiments, activating the positive regulation system includes releasing to the environment an activating signal, such as, an artificial molecule which is designed to activate the positive regulation system. For example, the activating signal may be arabinose, as exemplified hereinbelow (e.g. FIGS. 2B-2D), which when added to an environment that includes the pathogenic bacterium and the composition, activates the antibacterial activity of the non-pathogenic bacteria. Similarly, once removed, the activating molecule/entity is removed, the system becomes inactive. Hence, using an external controllable activating mechanism ensures that even if the system is present in food products, it is inactive and cannot be activated spontaneously.

In some embodiments, the infected organisms are marine organisms. In some embodiments, the marine organisms are within an aqueous environment. In some embodiments, the method is for disinfecting the aqueous environment. In some embodiments, the method includes adding the composition to the aqueous environment. In some embodiments, the aqueous environment includes salty water. In some embodiments, the aqueous environment is a farm for raising marine organisms. In some embodiments, the farm is in the sea. In some embodiments, the salty water is sea water. The term “adding the composition” as used herein, includes direct adding, such as, pouring the composition to the environment, and may also include indirect addition, such as, adding the composition to nutritional, therapeutic, or any other form of compositions added to the environment. Such composition may be commonly added periodically or occasionally, when required. Thus, in some embodiments, adding the composition to the aqueous environment comprises adding the composition to nutritional composition(s) used to feed the organisms raised in the aqueous environment. In some embodiments, adding the composition to the aqueous environment comprises adding the composition to another composition being added to the aqueous environment, periodically, or occasionally.

Thus, the method disclosed herein provide a biologic solution to the problem of pathogenic infections, such as, infection of aquaculture farm, infected livestock, infected plantations and the like.

In some embodiments, the aqueous environment is an aquaculture farm for culturing marine organisms, including, but not limited to, shrimp, fish and oysters.

The engineered non-pathogenic bacteria disclosed herein may be used to eliminate or prevent disease outbreak caused by specific bacteria. It may also be effective during shipments of plants, livestock and fresh marine products, including, shipment across long distances, and may be further used in restaurants which maintain, for example, live fish, oysters or shrimp.

In some embodiments, the composition is in a dry form. In some embodiments, the composition is a bacterial culture.

In some embodiments, the external signal (cue) activating the positive regulator is a bacterial pathogen, or a molecule derived therefrom, present within the infected environment.

In some embodiments, the composition is a dry composition, the infected environment is an aqueous environment, wherein upon contact between the dry composition and the aqueous environment, the bacteria are resuscitated and act as scouts or sentinels on the lookout for pathogenic bacteria. In certain configurations, upon identifying a niche with high density of pathogens, the engineered antibacterial bacteria are activated and eliminate the identified pathogens.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1: Generation of Non-Pathogenic Antibacterial Bacteria

A large gene cluster encoding an antibacterial T6SS1 (vp1386-vp1420, namely, including the vp1407 regulator and the endogenous effectors) derived from the marine pathogen Vibrio parahaemolyticus strain RIMD 2210633 was subcloned onto a plasmid, termed pT6SS1. This platform is not the inducible effectorless platform and is not intended to exemplify the engineered system, but rather to demonstrate that T6SS can be active inside V. natriegens.

The plasmid was introduced into the marine bacterium Vibrio natriegens (https://www.atcc.org/products/all/14048.aspx). V. natriegens is considered to be a “safe” bacterium that does not possess any known major virulence determinant nor an endogenous T6SS.

This transformed V. natriegens into a bacterium with antibacterial properties, which was able to kill competing Gram-negative bacteria such as, but not limited to, V. parahaemolyticus (other than RIMD 2210633) or E. coli. As illustrated in FIG. 1C and shown in FIGS. 1D and 1E, the plasmid-borne VpT6SS1 was functional in V. natriegens under warm marine-like conditions, as evident by secretion of the hallmark secreted component, VgrG1 (FIG. 1D) and by killing the parental V. natriegens prey. In brief, FIG. 1D shows the expression (in cells) and secretion (to the culture media) of VgrG1 from V. parahaemolyticus RIMD 2210633 derivative PORI (Vpara; T6SS1⁺) and its T6SS1 mutant (Vpara/Δhcp1), and from V. natriegens (Vnat) carrying the aforementioned T6SS-carrying plasmid (pT6SS1) or an empty vector control (pEmpty). Samples were treated (+) or not (−) with 20 μM phenamil to activate surface sensing in media containing 3% NaCl at 30° C. for 5 h. Loading control (LC) is shown for total protein lysates, arrows denote bands corresponding to VgrG1 and asterisks denote non-specific bands detected in Vnat samples.

The sequence encoding the native T6SS1 derived from the marine pathogen Vibrio parahaemolyticus strain RIMD 2210633 is provided as SEQ ID NO: 2.

TABLE 1
Sequences corresponding to SEQ ID Nos.

| SEQ | |
|---|---|
| ID NO: | Sequence name |
| 1 | Genetically modified T6SS devoid of vp1407 |
| | and of effector-immunity pairs achieved through |
| | deletion of operon vp1388-90 and introduction of |
| | deactivating mutations in the active site of the |
| | effector VP1415 |
| 2 | T6SS1 derived from V. parahaemolyticus |
| | strain RIMD 2210633 |
| 3 | T6SS1 vp1407 |
| 4 | T6SS1 3-gene operon vp1409-1407 of T6SS1 |
| 5 | T6SS1 antibacterial effector-encoding genes-vp1388-90 |
| 6 | T6SS1 antibacterial effector-encoding |
| | genes-vp1415 (including mutation |
| | in the catalytic AHH to AAA, denoted in bold) |
| 7 | Exogenous effector and immunity |
| | pair Tme/i1 ^{BB22OP}(C-terminal linker + |
| | Myc-His6 sequence, denoted in bold) |
| 8 | Exogenous effector and immunity |
| | pair VPA1263/Vti2^{RIMD 2210633} (linker + |
| | C-terminal Myc-His6 sequence, denoted in bold) |
| 9 | Exogenous effector and immunity pair Va02265/0^{12G01} |
| 10 | Signal receptor/regulator AraC gene |
| 11 | Gene cassette: signal receptor/ |
| | regulator AraC gene, AraC-responsive |
| | promoter, and gene cluster activator vp1407 |
| 12 | Gene cassette: signal receptor/ |
| | regulator AraC gene, AraC-responsive |
| | promoter, and 3-gene cluster activator vp1409-1407 |

### Example 2: Inducible Regulation of the Non-Pathogenic Antibacterial Bacteria

VpT6SS1 was transformed in V. natriegens into an inducible system controlled by an external cue, using a gene that serves as an on/off switch for VpT6SS1-mediated antibacterial activity. Three major positive regulators of VpT6SS1 have been identified previously in V. parahaemolyticus and were considered possible candidates for serving as on/off switch: TfoY, which is encoded outside of the VpT6SS1 cluster, and two regulators encoded within the cluster, VP1407 and VP1391. Following a combination of quantitative real-time PCR (RT-PCR), interbacterial competitions, and VgrG1 secretion assays, VP 1407 was chosen to function as the on/off switch.

By deleting the gene vp1407 (SEQ ID NO: 3), a positive regulator that is encoded within the T6SS1 gene cluster and is responsible for activating, either directly or via a second regulator, VP1391, the expression of all of its operons, the generated genetically engineered T6SS1 system was no longer active. As shown in FIGS. 2A and 2B, removal of vp1407 or vp1409-7 from the plasmid-borne VpT6SS1 (resulting in pT6SS1^{Ind}) rendered the system significantly (P<0.05) inactive as it lost its ability to mediate interbacterial intoxication (FIG. 2A) and to express VgrG1 (FIG. 2B, no arabinose).

Expression of vp1407, alone or as part of the 3-gene operon vp1409-1407, was found to activate the antibacterial T6SS platform. In fact, inducible expression (currently via the AraC-assisted arabinose-inducible promoter, Pbad) of vp1407 and of vp1409-7 (SEQ ID NOs: 3 and 4, respectively; and Table 1) introduced into V. natriegens were able to activate the T6SS platform in the non-pathogenic bacteria as illustrated in FIG. 2C and further demonstrated in FIG. 2D. In brief, vp1407 or vp1409-7 3 gene cassette were engineered into the V. natriegens chromosome (replacing the dns genomic locus) under regulation of the arabinose-inducible Pbad promoter together with its constitutively expressed regulator, AraC (the derivative strain in which only vp1407 was used was termed Vnat^{Reg}; FIG. 2C). As shown in FIG. 2B, VgrG1 expression and secretion were restored when either V. natriegens strains carrying pT6SS1^{Ind} (corresponding to the strains presented in FIG. 2D) were grown in the presence of arabinose. Furthermore, VpT6SS1-mediated antibacterial toxicity was also restored upon arabinose addition (FIG. 2D—circles) and not in the absence of arabinose (FIG. 2D—upward triangles), compared to time zero (FIG. 2D—downward triangles). Notably, the reduction in prey viability that was mediated by this inducible system (˜3 orders of magnitude; FIG. 2D) was more pronounced than the reduction mediated by the natural VpT6SS1 gene cluster in V. natriegens (˜50-fold; FIG. 1E, discussed above); these data reveal that external activation of the system results in potent antibacterial activity. Together, the above results indicate that VP1407 can serve as an on/off switch for VpT6SS1.

It is noted however, that the control of vp1407 or vp1409-7 expression can be replaced to respond to any other internal or external cues (signals) by swapping the promoter region and upstream regulator, thus making the platform customizable and responsive to its surroundings.

### Example 3: Activity of the Non-Pathogenic Bacteria Under a Wide Range of Temperatures

The inducible platform provided the non-pathogenic bacterium with controllable antibacterial activity against other Gram-negative bacteria at temperatures ranging from 20-37° C., with an optimum at 28-30° C., as detailed below.

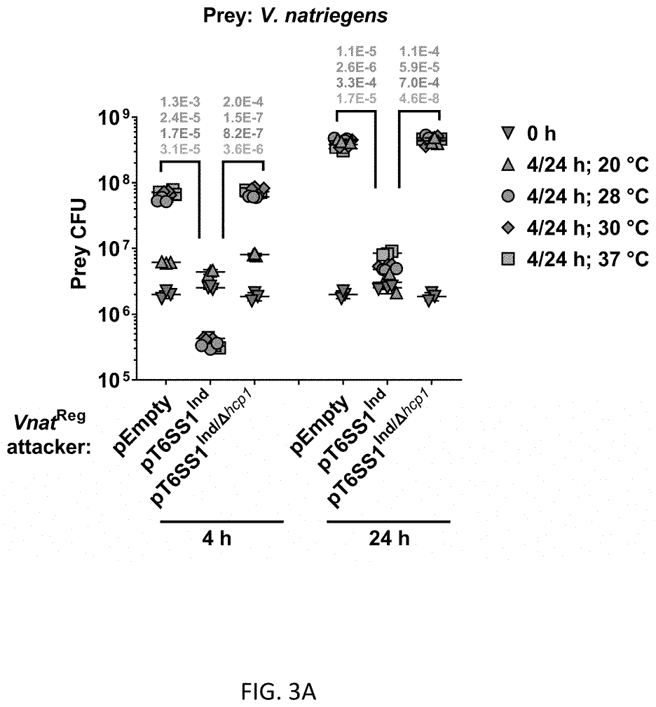

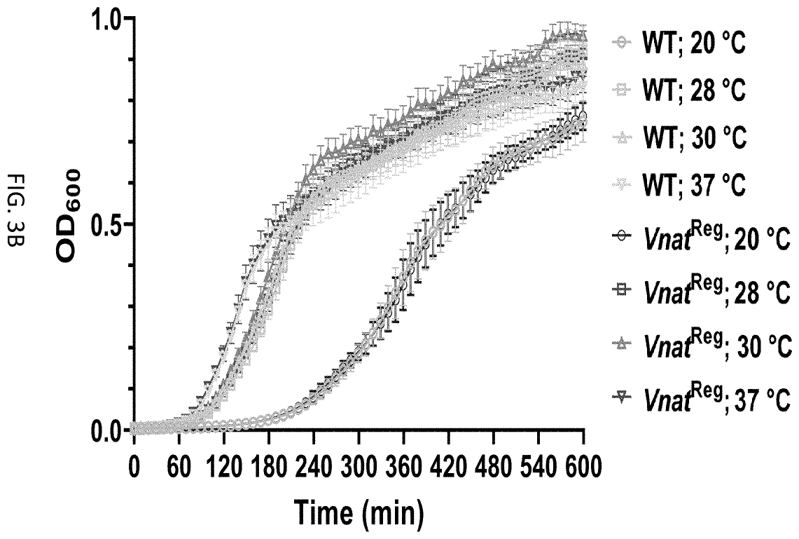

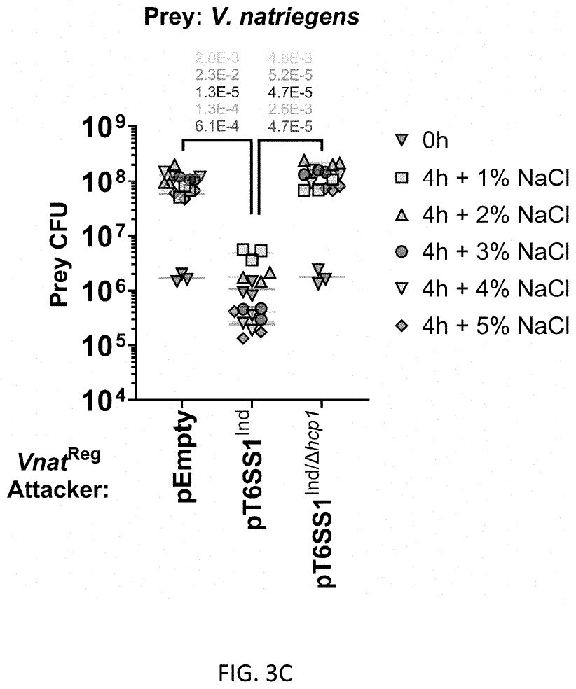

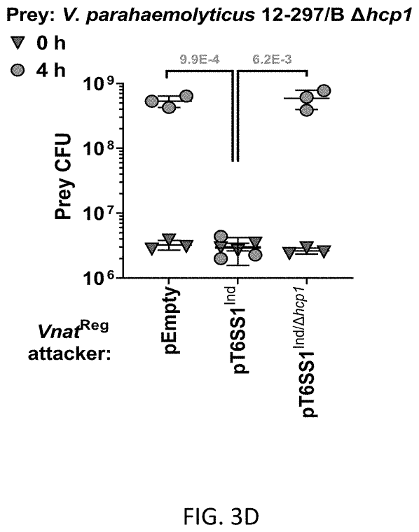

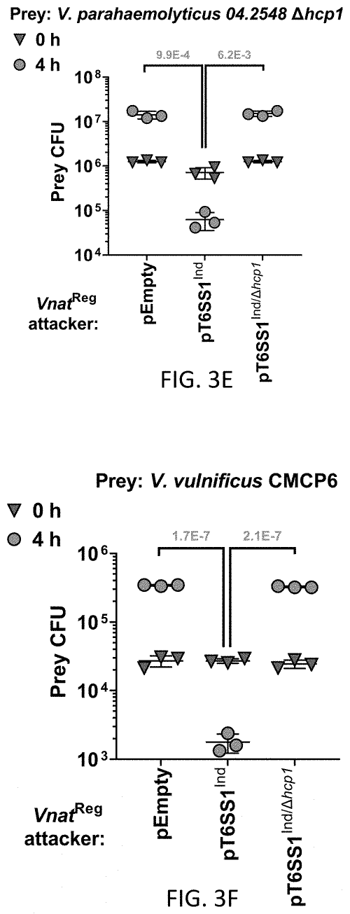

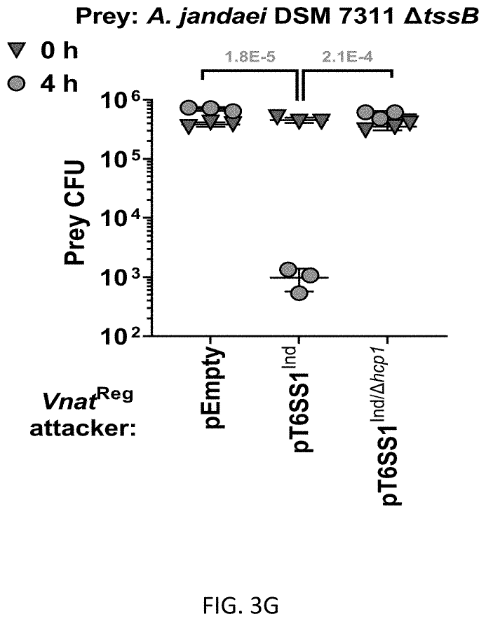

Using parental V. natriegens as prey, prey viability after 4 and 24 hours of competition with Vnat^{Reg} attackers carrying an inducible T6SS (pT6SS1^{Ind}) or its inactive version (pT6SS1^{Ind/Δhcp1}), was monitored at temperatures ranging from 20 to 37° C. As shown in FIG. 3A, the system was active within the tested range, although at 20° C. it required 24 h to mediate an effect similar to that seen at other temperatures within 4 h. This was probably due to slower growth of V. natriegens at this temperature (FIG. 3B).

V. natriegens is a marine bacterium, hence it can be used potentially as bio-treatment for intoxicating other marine bacteria. Aquaculture produce, such as shrimp, are often farmed at temperatures around 28° C., an optimum at which our platform functions well (FIG. 3A). The activity of the inducible system at the optimal temperature (28° C.) against a sensitive V. natriegens prey under varying salinities which can be found in aquaculture farms, namely, within a salinity range of 1-5% NaCl (w/v) has been tested. The system was found to be active throughout the tested salinity range, with an apparent preference for salinity of >2% NaCl (FIG. 3C). Thus, the antibacterial system is functional under a wide salinity range. Next, the ability of the antibacterial platform to intoxicate diverse marine pathogens at this temperature was tested (FIGS. 3D-3G). Indeed, under inducing marine-like conditions (i.e., 28° C., 3% NaCl, and in presence of arabinose), Vnat^{Reg} carrying an arabinose-inducible T6SS outcompeted pathogenic V. parahaemolyticus strains (the shrimp pathogen 12-297/B and the clinical isolate 04.2548; FIGS. 3D and 3E, respectively), as well as the pathogens V. vulnificus and Aeromonas jandaei (FIGS. 3F and 3G, respectively). Notably, to avoid masking the activity of the antibacterial platform by prey-mediated counterattacks, potential antibacterial T6SSs in few of the competing bacteria were inactivated by deleting genes encoding the conserved T6SS components hcp or tssB, as indicated (FIGS. 3D-3E and 3G).

### Example 4: Safety Mechanisms of the Non-Pathogenic Antibacterial Cell

The engineered non-pathogenic V. natriegens disclosed herein is capable of acquiring external DNA via horizontal gene transfer, raising concern that it may become virulent over time, if it acquires virulence traits from its victims. Another concern is that the modified T6SS system may find its way into another bacterium. The Δvp1407 T6SS1 cluster was designed to be encoded on a different genomic location from that of the operon, e.g. it may be encoded on a plasmid, while the arabinose-inducible vp1409-1407 operon, or only the arabinose-inducible vp1407 is inserted into the V. natriegens chromosome, as exemplified herein. In addition, the presented examples of diversifying effectors used to provide toxicity to the effectorless platform were introduced on a second plasmid; however, they can also be inserted into the chromosome. This separation ensures that even if this system finds its way into another bacterium, the other bacterium will not be able to use it as an antibacterial weapon since a required regulator and its corresponding effector repertoire are encoded elsewhere on the V. natriegens genome. To prevent V. natriegens from acquiring external DNA and become virulent over time the tfoX, a conserved regulatory component of the DNA uptake machinery and natural transformation, is deleted.

### Example 5. Manipulating the Effector Repertoire of the Engineered Platform

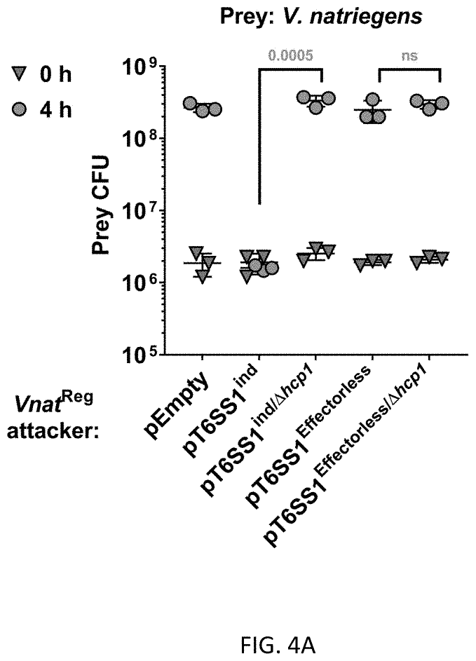

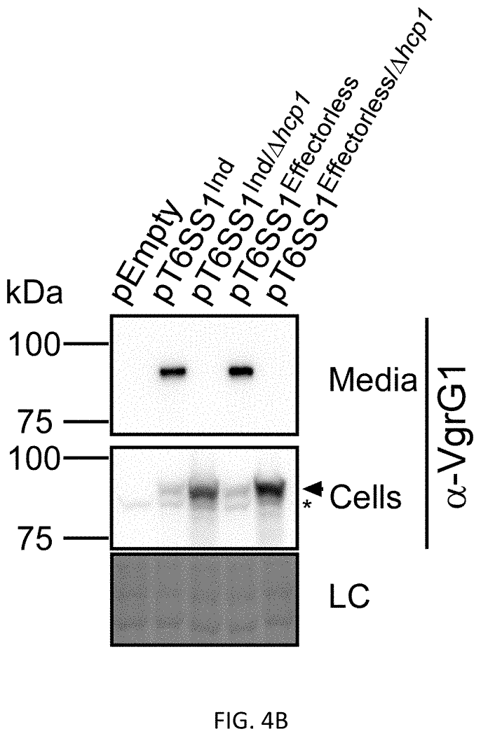

The goal of this study was to exemplify the ability to operate the system under various toxins that the system deploys. The inducible T6SS platform disclosed herein carries two endogenous antibacterial effector and immunity modules, one at each end of the cluster (vp1388-90 and vp1415-6), which mediate the intoxication of a wide range of bacteria (see, for example, FIG. 1E). To enable control of the effector repertoire, the platform was initially cured of its endogenous effectors. Therefore, a version of pT6SS1^{Ind} was constructed, in which vp1388-90 (SEQ ID NO: 5) have been deleted and the two histidine residues in the putative active site of the AHH toxin domain (residues 563-4), which is fused to the PAAR repeat-containing domain in VP1415, have been substituted with alanine (SEQ ID NO: 6; hereafter referred to as pT6SS1^{Effectorless}). As expected, this inducible and effectorless platform was unable to mediate arabinose-inducible intoxication of a sensitive prey (FIG. 4A). Importantly, the effectorless T6SS remained functional, as evident by the T6SS-mediated secretion of VgrG1 (FIG. 4B).

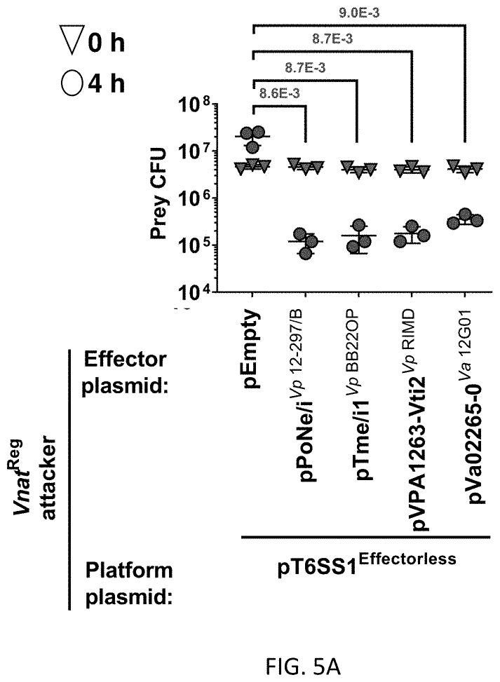

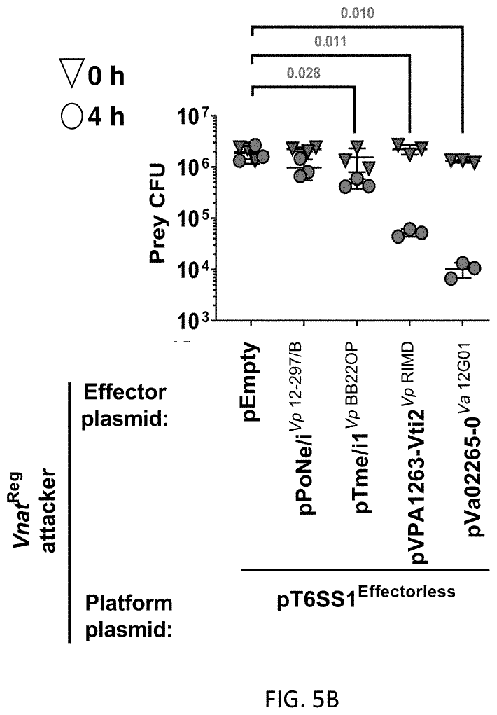

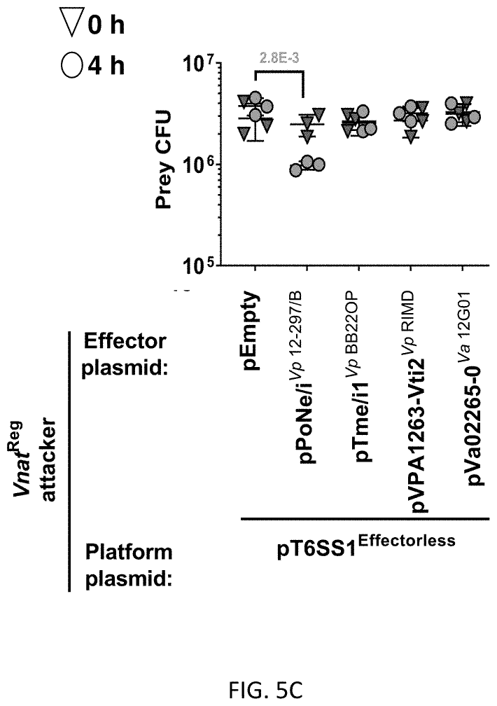

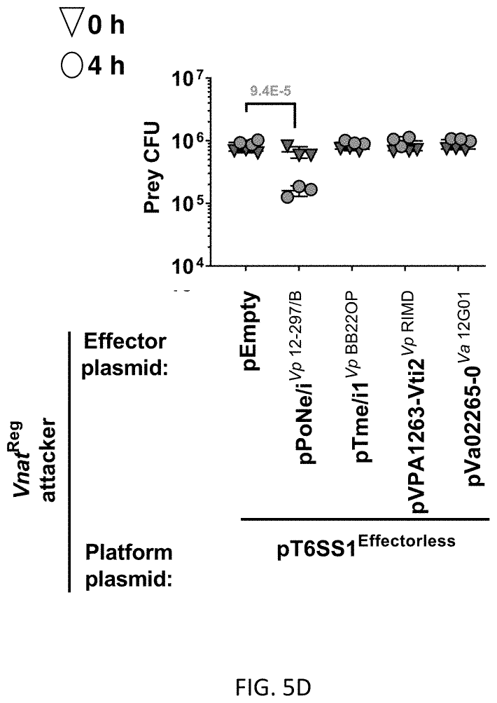

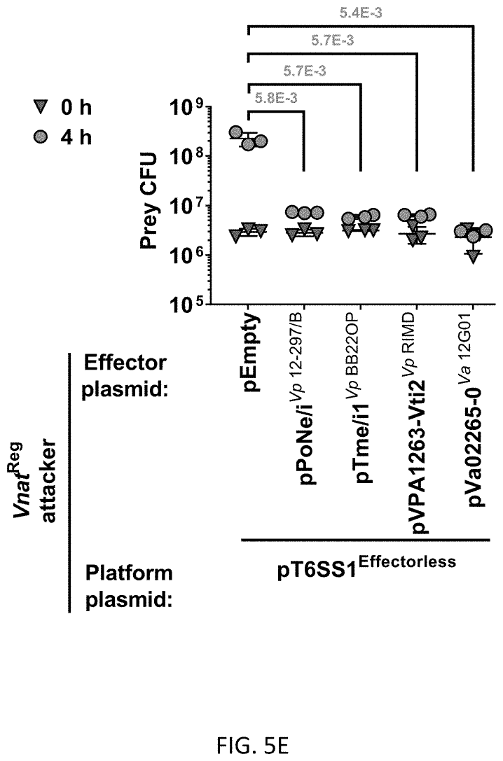

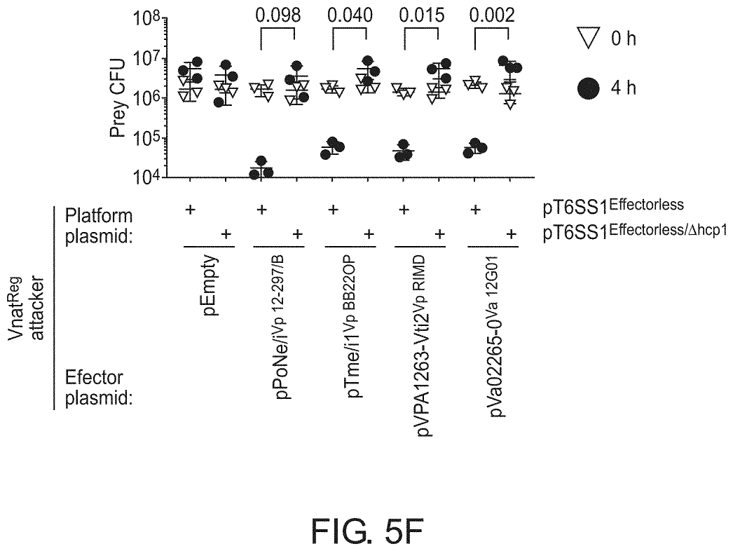

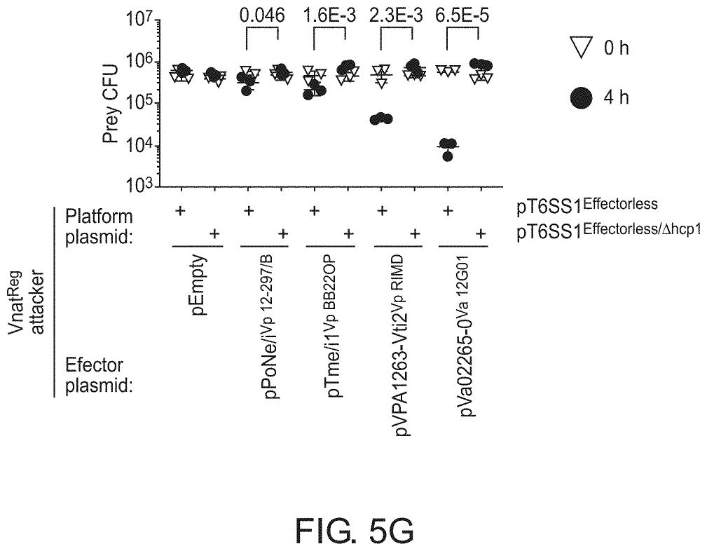

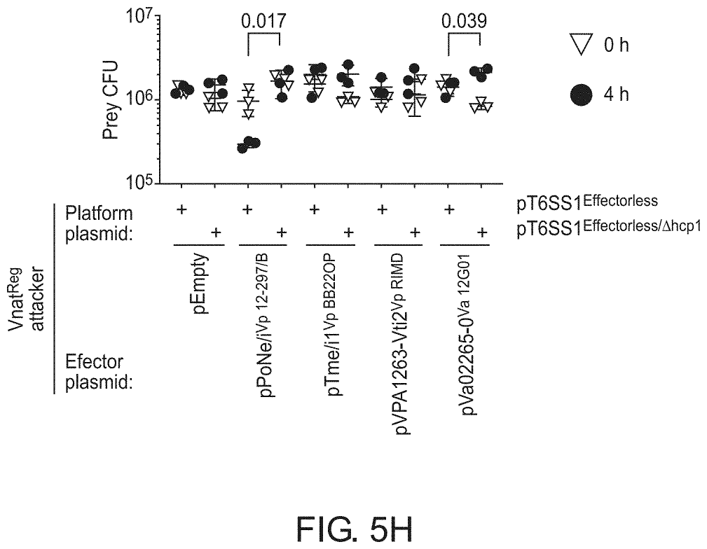

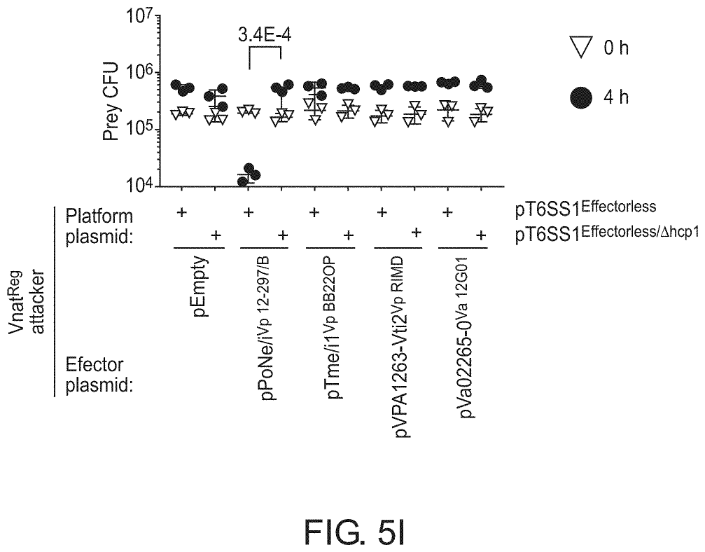

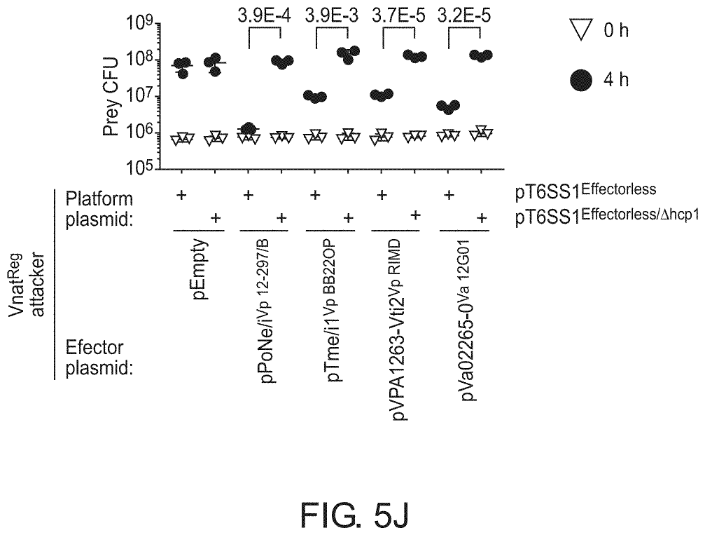

Next, plasmids carrying T6SS effector and immunity modules were introduced into Vnat^{Reg} harboring the inducible and effectorless T6SS platform various expression. These modules were shown to be secreted by VpT6SS1 homologous systems (PoNe/i from V. parahaemolyticus 12-297/B together with VgrG1b, Tme/i1 from V. parahaemolyticus BB22OP, VPA1263-Vti2 from V. parahaemolyticus RIMD 2210633, and Va02265-0 from V. alginolyticus 12G01); these modules were placed under Pbad regulation. As expected, the exogenous effector and immunity modules restored the platform's ability to intoxicate parental V. natriegens prey. Surprisingly, however, each module differentially affected other bacteria that was used as prey (FIGS. 5A-5E). PoNe/i intoxicated all of the tested prey apart from A. jandaei, whereas the other three modules affected aquatic prey (i.e., V. natriegens, V. vulnificus, and A. jandaei) but not gut-residing bacteria (i.e., E. coli and Salmonella enterica; FIGS. 5D and 5C, respectively). Interestingly, Tme/i1 had a major effect on vibrios, but only a minor effect on A. jandaei viability. These results show that the effector repertoire of the VpT6SS1-based platform can be manipulated. These results also reveal that effectors have different toxicity ranges.

Next, it was determined whether the differential toxicity of the tested effectors can enable selective targeting of specific bacteria within a mixed population. Indeed, the same phenomenon was observed when the five tested prey strains were all mixed together and competed against the antibacterial platform (FIGS. 5F-5J). The results indicate that natural, exogenously expressed effectors may be used to target specific bacteria within a diverse community.

### Example 6. Deploying Multiple Effectors by the Engineered Platform

To demonstrate that the engineered platform can deliver multiple exogenous effectors, and to determine the advantage of deploying multiple effectors to widen the platform's target range, plasmids expressing a combination of two effectors, VPA1263 from V. parahaemolyticus RIMD 2210633 and Va02265 from V. alginolyticus 12G01, were engineered, together with their cognate immunity genes to prevent self-intoxication. The resulting plasmid was termed pVPA1263-Vti2+Va02265-0.

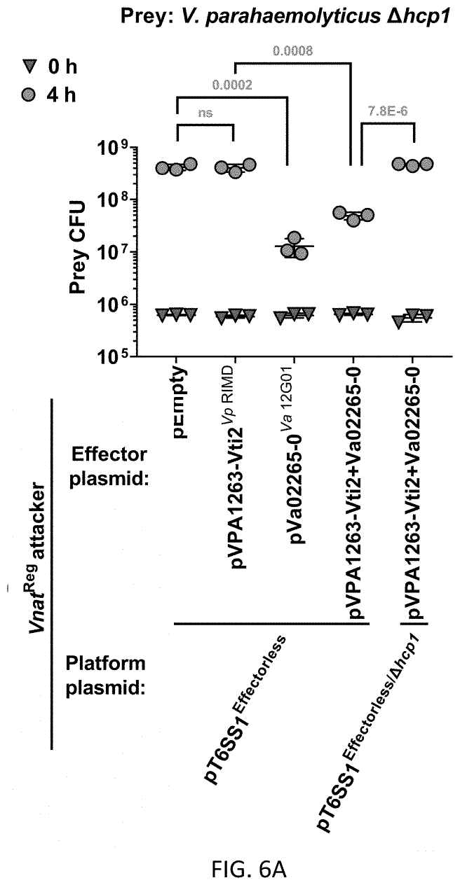

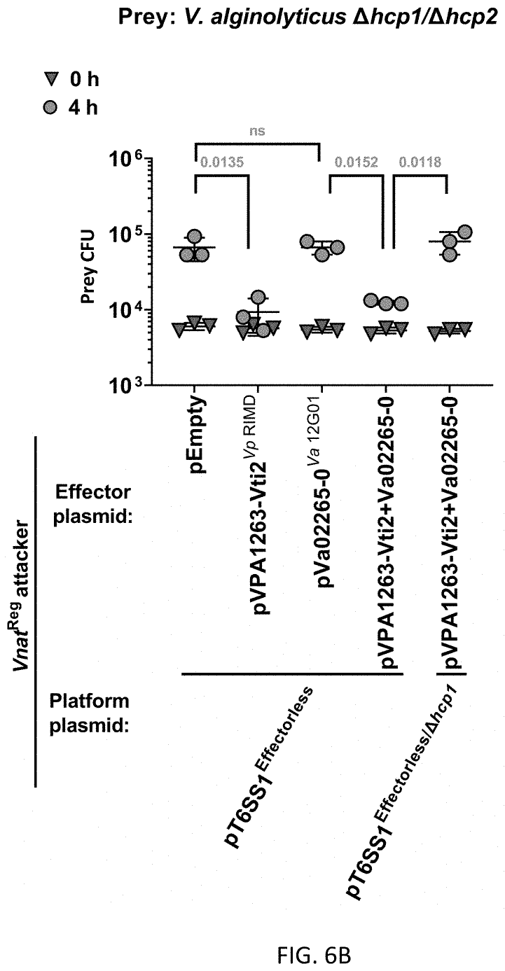

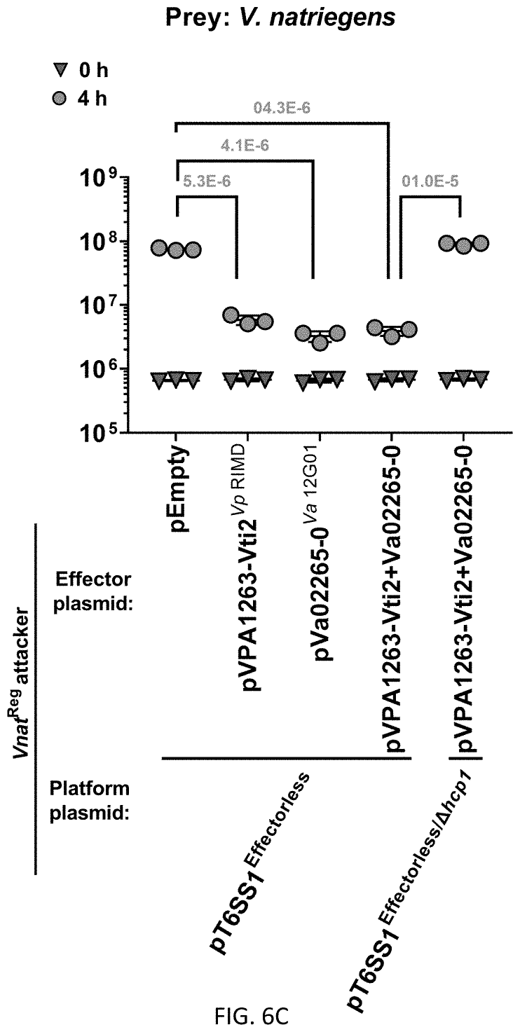

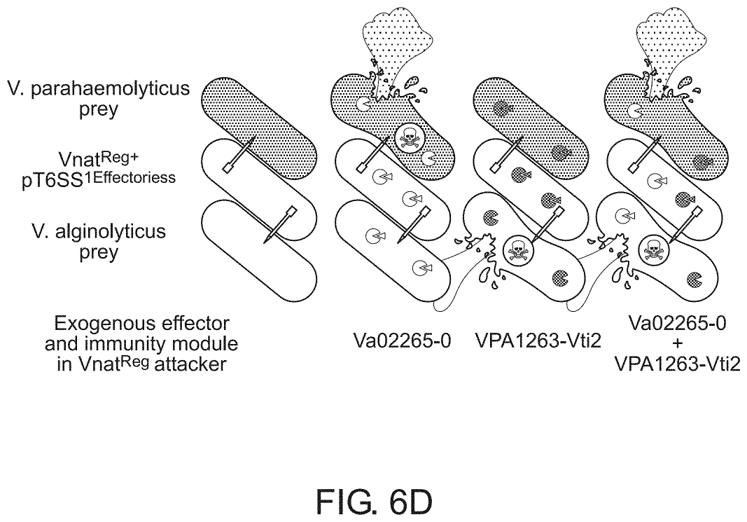

As expected, single effectors were able to mediate intoxication of a prey that was not the strain from which they were derived. Nevertheless, their combination mediated T6SS-dependent intoxication of both prey strains, as illustrated in FIG. 6D and exemplified in FIG. 6A-6B. Notably, antibacterial T6SSs in the tested prey strains were inactivated to prevent counterattacks that could mask the effect of the engineered platform. Surprisingly, combining the two effector and immunity modules did not provide a significant advantage over a single module, when deployed against a prey that is sensitive to both effectors (i.e., V. natriegens; FIG. 6C). The results demonstrate the ability of the engineered platform to deploy multiple effectors, as well as the applicability of using multiple effectors to widen its toxicity range.

### Example 7. Co-Colonization of V. natriegens and the Marine Pathogen, V. parahaemolyticus, in the Gut of a Marine Animal

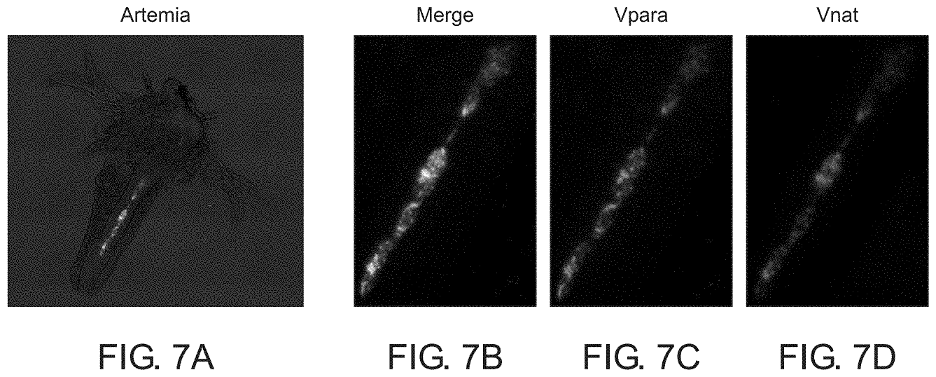

To test whether V. natriegens can colonize the intestinal tract of marine animals, and therefore be used as an antibacterial treatment in aquatic settings, nauplii of the aquatic crustacean, Artemia, were subjected to an immersion challenge assay. After hatching in artificial sea water, Artemia were transferred into 96 well plates, one Artemia per well, and were immersed in 10⁷ cfu/ml V. parahaemolyticus and V. natriegens strains constitutively expressing GFP and RFP, respectively, to allow for their visualization. After 24 hours at 28° C., the Artemia were placed on a glass cover slip and visualized under a fluorescence microscope (FIGS. 7A-7D). FIG. 7A shows an Artemia with the presence of both bacteria in its intestinal tract, FIGS. 7B-D show only fluorescence signals within the intestinal tract of the Artemia presented in FIG. 7A, where the image in FIG. 7B shows GFP and RFP fluorescence (corresponding to both V. parahaemolyticus and V. natriegens strains, respectively) and FIGS. 7C and 7D show separately each of the GFP and RFP fluorescence, respectively. As shown in FIG. 7A-7D both bacteria clearly co-inhabited the digestive tract of Artemia and were in close proximity to one another.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1.-33. (canceled)

34. A genetically engineered non-pathogenic bacterium having antibacterial activity, comprising a gene cluster encoding an antibacterial protein delivery platform for producing antibacterial activity, operably linked to a signal-inducible positive regulation system, and at least one effector-immunity pair, delivery of which exerts the antibacterial activity wherein the gene cluster and the positive regulation system are localized in different genomic loci within the non-pathogenic bacterium.

35. The genetically engineered non-pathogenic bacterium according to claim 34, wherein the positive regulation system comprises at least one activator associated with at least one promoter, configured to activate the antibacterial protein delivery platform to produce the antibacterial activity, wherein the at least one activator is a signal receptor/regulator configured to associate with at least one signal activated promoter, upon sensing an activating signal.

36. The genetically engineered non-pathogenic bacterium according to claim 35, wherein the positive regulation system further comprises a plurality of regulators, each associated with at least one promotor; and/or wherein the gene cluster, the signal receptor/regulator and the at least one effector-immunity pair are localized in different genomic loci within the non-pathogenic bacterium; and/or wherein the activating signal is an external signal exerted by a pathogen.

37. The genetically engineered non-pathogenic bacterium according to claim 36, wherein the positive regulation system further comprises at least one gene cluster activator, and wherein the at least one signal activated promoter induces expression of the at least one gene cluster activator, which upon association with at least one gene cluster promoter within the gene cluster, activates the antibacterial protein delivery platform to produce antibacterial activity.

38. The genetically engineered non-pathogenic bacterium according to claim 37, wherein the gene cluster, the signal receptor/regulator, the at least one gene cluster activator and the at least one effector-immunity pair are localized in different genomic loci within the non-pathogenic bacterium.

39. The genetically engineered non-pathogenic bacterium according to claim 34, wherein the at least one effector-immunity pair and the gene cluster are derived from same or different bacterial strain.

40. The genetically engineered non-pathogenic bacterium according to claim 34, wherein the gene cluster is encoding an antibacterial Type VI secretion system (T6SS) devoid of a T6SS positive regulation system and T6SS effectors-immunity pairs; and/or wherein the gene cluster is derived from a pathogen.

41. The genetically engineered non-pathogenic bacterium according to claim 34, wherein the bacterial strain from which the genetically engineered non-pathogenic bacterium is derived, is a marine bacterium and/or is devoid of an endogenous T6SS.

42. The non-pathogenic bacterium according to claim 34, wherein the gene cluster is located on a plasmid.

43. The non-pathogenic bacterium according to claim 42, wherein the at least one effector-immunity pair is located on a chromosome.

44. The non-pathogenic bacterium according to claim 34, wherein the gene cluster and the at least one effector-immunity pair are located on the same or different chromosomes in the non-pathogenic bacterium.

45. A composition comprising the non-pathogenic bacterium according to claim 34 and a carrier; the composition being in a dry, lyophilized form, or in a bacteria culture form.

46. A method of treating pathogen-infected organisms, the method comprising:
a. contacting the infected organisms with a composition comprising the non-pathogenic bacterium of claim 34, wherein the at least one effector-immunity pair is capable of reducing or eliminating activity or proliferation of the pathogens; and
b. activating the positive regulation system using an activating signal, thereby inducing the non-pathogenic bacteria to exert antibacterial activity.

47. The method according to claim 46, wherein the infected organisms are marine organisms in an aqueous environment.

48. preferably wherein said contacting comprises adding the composition to the aqueous environment. The method according to claim 46, wherein the gene cluster is encoding an antibacterial Type VI secretion system (T6SS); wherein the T6SS is derived from marine pathogen.

49. The method according to claim 46, wherein said non-pathogenic bacterium is a marine bacterium and/or is devoid of an endogenous T6SS.

50. The method according to claim 46, wherein the gene cluster encoding the antibacterial protein delivery platform is derived from a pathogenic bacterium.

51. The method according to claim 46, wherein the gene cluster and the at least one effector-immunity pair are located on the same or different chromosomes in the non-pathogenic bacterium.

52. The method according to claim 46, wherein the activating signal is an external signal; and
wherein the external signal is a bacterial pathogen, or a molecule derived therefrom, present within the aqueous environment; or
wherein the external signal is a compound added to the aqueous environment, in order to activate the antibacterial protein delivery platform.

53. A kit comprising at least one receptacle containing the composition of claim 49, for treating infected organisms and environments, and comprising instructions for use; and further comprising a positive control configured to verify that the composition in the at least one receptacle, is active, wherein the positive control comprises a sample of an activating signal molecule and a detector of secreted platform components.


SEQUENCE LISTING (12 sequences)

SEQ ID NO: 1 - DNA, 48928 bp, synthetic construct
  misc_feature 1..48928: note = Genetically modified T6SS devoid of vp1407 and ofeffector-immunity pairs achieved through deletion of operonvp1388-90 and introduction of deactivating mutations in theactive site of the effector VP1415
  source 1..48928: mol_type = other DNA
agtgagcagataatgactaaaagatacgcctgcaataatagtgagtgcgatggtaataagaggcagagcaaggttatgattacgtgaacttaatattaaacttgtcattaaatgaaaactgtgcttagaagctgaaaattgaggggagtataatatttaaaataaaatcttacacgtcactaacgacaattcctttgtttttactaactaaataatgatagatatgaaatagcactaagaatagggttgaaaagtgtctgtttttttagtttttttgctttattttatggttattttgtcgcgttgagaaaattccacacaataatataatctcggcaaaaatatactctgccagattattatgttcactaaaaatacgctaaaatcaacgttaggtctcatggctacttcactattattgagtgcatgtggcggaggggacggtggcgataatacgccacctgcgaaggaatacaggaaccatctcaggtaatgtttttgacgctccagtcaatggtgcaaaaattgaggtgtttgagtttaaagatggaaagctaggaaggaagctagcgagcacatcttccaatgcatttggtgattacaaagtcgaatttgaatcgtcttcgatgccattgtatgtcgtcgcacaacaaggtagttacaccgatcctttaacaaaagagattgtgtcctcgagtgttggtaaaacgttgcgtttagaaggcgtggtgaacttcagtgaaggtagtaaccaaaagttgatgttgacgcctctgaccaatattgtttccggtttcgcaaaatataaaattgcaggcggcgtgtctggtccggacgctgtctctcaagctcttgatagtattaatggcatgtacggttttgatgtcaatgaaaccacaccaattgatattactaaaggtgggcagagtagctttgcaacgccggggcatcagtatggtgcattgcttactgcatactcttcttactcttatgacatgatccaaaggtacggtcaatccgaagacaatgtatatacatcaatgcatttagctgatgtacagtttcgtgatgtcattgcagacggccttcttgatggcctagaaataagcgaagcaacgggagctgtcacaccacttacatttggtcagcagaaaatcaccagtgatgtgtacactcaagaactctctcagcaagttttgatagttgtaaatgaccctacactcaacatttcaggaacaaacgctgacgattacgttgcctttagccaacaaataaatagccttggaaccgctggcgaaacagatggtgtcattccacctcgtgatgacacagatattgataccattccgccaactgcaactcgaacagacaatgatgtattggcaggtaaagacatcgtagatatcaaaatcaatgacgatattggtgttgagtcagtgtctgcatttgttgaatacaaactcaacggtgcatggcaaggtgagtttcaatgtaatgatcagctgacgtcaggcagtaagttttgttcggtggatgcgcaagggtttgaggttggtcttcgtgaaacgaacatcaaagtaatcattgatacaaaagcgatcgatgctgtttatgtcgaccaagaaactggcctttccaaggttactgctgcacgtttagtcttatacactgcagatgtgctcggaaataaattgcttcctggcggtgtaaaggtcattatgtcaatttctcttgggataacgacgcgccagttatcaccgtgacctcgtctagcgctatcaataatgagcttcaagaatatgtacttaaaggtcttgtaaaagaagcgtctcaggacattaagtccgtcagtgtgtcatttaagggtggtttgcctgacgatgttgcttgttctcccgttacgtcagagtcgggaagtgcctgtgaatttagcaaagcgtatttaacagaccagttcttatcaactaccacgtttgatataaaagccactgatacaaaagacaatgtcggtgagtttcaacatgcggtgagccgagatgatcaagcacctgcacagatcatcacatacccggaaggtacgcccatgagttacgtcaacgtgagccttaatggagaacgaaccacttacgagggcgtatattcacaagatacttatacagctgataacgtgcagtccagccgagattacttgaaaatagattatgtctatgcaagcgcaggtcttaaatccattagtgggattgatttcgcaaacttcaatgttaacttattgaaagaaaaaaagattccttacgttcgtgtcaaaatatctgatgcgagttcagagactgtattaggttcaagtgctgacaaacttaagctcgtcgtgaagtattacgtaagtcaaaacaatgacaacaactatgcgttccaaaatacaacgcagacgttagcttcgacggacgccataaccgcaagcattcctcacgaaaccattttggatagtagcggacgagttgatgaagttatttactatgtcccttttgtaaaggagatcctaggtgatggcttcaaaaacgtctcagaaaacgcgagccaaaaactggtaattcaagctattgatgagtcagagaatgaaagcacgactcaagaagtttatttcagaagcagtttcgacttgccgacgatgactatcgtgacacctttcattggtgcacgtgttcaattggaaggtttaaaccctaacggagagtttacgtcattagctagttgctccacgattcagcaaccggaaagtgatgagtctgccgcgtttgatgttgcttcatgtgagactacaaccgatgttgttaattatgattttatgcgagttcgcttaattggtgttgatggaactgatccgcattactatcaatggaaggataataaaagcgcaagagctagcgttaacttaaacgatgcaaatataggcgcatacttcgaactgaacggttcgaatacttattatgttacggagttatctacttaccaaacaggcctatttgactatcgctggaatcaagttgaggccggagacaaaacctcagagcgcgcggctctcattcttacggatgtgaagtatgcattgtccggtggaaacgattcatttttcggttttgatccaacagtgacatcttatgcgactaacgagatgttagaaaacccaattccagatgaactgtccaacgactacttgcatcgattcctcgttgaagcgatcgatgatttagctcaagacacgccacgtaataactcttctgattttgcttctgctatctatgatgatctgagctatgacggtaaggcaaacggtattggatctggaggtaatctaattaaattagatagttatgagtttagttctgacacttaccgtaaagatctcgctcagtcttactacaacatcatgacggaaaaatacggaattccaaccaatattgcccagctttatgctgacgatatatcgcaggcaaatccaaagctagacgaagacgctatttttgatagtgagggagaaagtatagataaagaagcgcctaaacaaacgttaacaatagagactggccgagaaactatggtcggcaacaaacgttacgtagcaggcgaagttgtttctaaaattgtcttggaagatccatcaggtatcgttgaaaatacggaaaatgcgcctacattcaagagcatgtggtatgcacagagcaagcctaataccgcgataccattggaactgagaattgatgagtctggcaacagcaataaattccgcaaagagtatttgtttacactcaatacgcaatcaacagacctaatagacatcatcaagtttgctctggagacaacggcggtcgatacccaaggtaacgcttataccgatctaaacccatatattgaaactctatatgttgataatgactatcccgcagcgacttacattccaccaaacgacttggaagggaacccaattggcgaggatatttatttaaatgcgaattcagttcatgagttgacatttaaacttcaagatcttgtgggggaccaactggataagcggagtttatcgtttgtcgatctctcaaatggtacaacggtcgattacacaagcgatcagttttccagtaatacgcaagattcatttaaggttaaattatgtacaggtactctttgtgcgagccaaggtaaagtcgtttatccgggagatggtaactggactgtcaatgtaaatgcagaagacaatttggggaactttgttaatcgtcaaactacaaattcaccaaaattcaatatcttaattgactctgaagggccgattgttaatggaggaacaatcaataagcgtttgggtggaaactcatcttgggtgccggatatttcttggggtaacttgagtccaggcagtgtagttgaaattgatatgaagacggagtttggtaagcgtattactttgcaacattatgagactgaacctacagacagtgtggagccttatctattcggagagcaaccaaacattgaggttcgtttaatagccggggcgtttaagtatgaagagacgaactcgttttacgtaaaagcgaaagacagtgcttaccctcaagcagagtcgacaagcgaatttgtttttcaagtagacaacaaaggcccggtaattgaactgtctgaaccttggatcgtagacaaaaactctgctggtaactacgtcctgggacctgattttcaagtgaagctgagttcagtcattgatgattctaaagtaagtaacgtcgagctgtggcaacaagctggtgactctgcaatacagtcaattgtacctagagatccaacacaaccatttgaaatggaaattggtcgtatacaaagtgacagtatcaccattgatgcaaataagagggcgaacctgttcattaaggcgatagatgagtatggattcgaatctcagacagagccacgaacgataattattgaccgagatggtcctacgttaactctgaatgggtttaactcggaaagttattatcgtggtaactacgtgttttcactttcagctcaagatctgagcgaaacgggaacgccttcatcaaatggtgtgaacaagacgacattggaatattggagctttatcgatacaccttctgataccggcacacaagtagatgaagatatgaaagtgagcttagaaggacagaatgatggtgatagttcaacccgtacaatcaaactcaggggcagcgatgttcgcgggaatacgacagaggtagattacgcgattaaagtgcaaaacaagttgccaacaattcatagtgtgtctttttcctatgaagatggaacgccgattactggtgggttagttacgcgcaacgatcctgttgttattacccttgatgcggaagatgcaagtggaattcctcaaatcgacggcacttatagatacggcgatcaaaccaataatatacagtttagtgaagatgaaacgggtaagtggaagggaagactaacttctgcagaattatctcaggatggaagctatgaactaaagttcaaagtttataacaatgttttgtataactcaggcgaaacacggccagtagtagaacgttcggaaagtattaatgttcaacgcgaaggggtcgtgttatctgtcgcgcaacctgaaaattttcaggctcatatctcgggtaagacgttgagtgtcgagttcaaccctgtgggtgatgttaaggctcggactctagaatgttgggtaagagaaaattacacctcaagcgaagctccaaatgatggattgagcaatgcttactctggcgttatcactaatccacaggaaagttataagtgtagtgtgacgacagatgtaaacatgtctactgcacctgtcgttctaattactaggacagtgggccagaacgatacagagacggtgagcaaattcagctttaatatgatggatattgaagcaccagcagcattagaggatacctatctatttacaggtaacgaagttgaatttgatgccgatggaaacaaaatgttgagcttaacattgagcttcaaagataacttatcaggggtgaatactgggccaacgaaacgaccgttgttagttagaaaccgttttaatataacgtttgaaccaaaaagttgtagcgtgagtgctggggttactacgtgtacttacaccgaacgttattcggatatcatcgcaacgggggtatcacaacatctatataccattaaaaatttatctgatatggcaggtaatacatcctcagatcattctctagaactattgttgccacctccaggttcagttgatattggaatcacaagcccaacggatgacagtatcattcgaggcgctcagctggtgatgaactttaaagtgaaggttggggcacaatcaattttagaaaatgtgattgcaacagtggatggggattcatataacctcaatgatcatccagggaacttttcacgatttaacgattgtggagagggctattcctgttcgacatttgaagcgcctctaagtgcgactttggatggacaagtgatcaaaccgagcatccaagctattgatgtgtggggagactcaagtagcgataacgtcagtgtgatggtcgataacagcaaaccaataattgatagtgacgctgttgtcagtgaatcccctacagattcaagcttggttaggttccagttttctattagggatgaaggaagcggtattgcaagcgtcaaatacacaaccgtcaaccctagccatgtttttaataaagaggaaaatgcagataactcacatctttatttcgagctgccaaaaagtgagctgcaagggctgaatcaaatccgagtaagcattgaagcaacggataaagtgggctggagtgaaagtaagactgtggatattgacgtgtctgtacctactattacggtctcatttaatggaatgactagcttgcaaggaggcaaactagcttttacaaaagaaagtcagttattcaatgtgacttcaaaagaaggacatgatgtaaaggcatcaaattactctatcgatttagttccaagtacaggtgatacgctaaatttttcagggaatatcgtctcatcgacagggtctgatactatggtttttgacgctgaagatcagaccgaatatatgtataaagtgacggtcaccgactcaatagggcgttctattacagactttgaactttttagtacaacatatggtgccaaaggcatcacatctatcgttgactacgagttaccagcaatatctggtgtttctgctgatcaagtatctatgacaccaacggacggtaaatatgatttagatgtaacagcgtatgttacagataaaaacttgagttcagtgacatctacagcagataacgggagtggggtaaaagtgggaccacaatcagttacagagcctgttagtgtagatgatccttatgtgatgcactactcattgactcctggtacctatacgctacaagtagccgcgttggatttggcgaaccataagactgagaaatcaacaagcgctaccgtaaaggcagcaaccgttccagagttaacaatatcaactgcatcacctagcccttcggctggtggagttgaaatcccgcttacatttactttttcagaagaggttaaagaatttagtttctccgatgtgaaattagaagcgagtgataaaaacgaagctggagagcttcagcaagtatcatggaacaccagtgacaatataacttggaccgttacctacacaactccagaaaagcaaaataaagaaataatcatacgggtcgacgatgatagttatgagagcgtcaactctatccccggaaaaggggatagtttgattttggaggtagaaggagtattaccaacgctgtcaaaagtgacttttgatcctaagcatcaagcgataggacagtcggtgaacatcacacttgagttcgataaggaagtgcaagaagcgacagcaaaactgggaaataatgatgttacttcactcgttgctacagcgaataagaaaatttggacggggagggctgaagtccccaatattccagaactcaatgtcggcctatttgtaagtaagtacaaagacttgattggtaatatgggcgaagatgattcttccaacgcaataccaatcacaccaaccttggcgatcacgcctgtaggcaatgtggatagcagcaatgccgccgcattgcaaatcaccggtacttcaagccgctttgatggccaaaccgtgagcgtagaaatcaaagcgcaaggcagtgaaacggttatcgccagcggcagtgccacggtacaaagtggtggcgcttggacgagcaacgcgatggacatcagtggtgagccaaatggcacctacaccgtcgttgtcacaggcaccaatgcctcaaatgtggaagcaacagaaagcacaaacttcacgttggcacaggcgttgccaacactgactaacgcaacattcaatccgacgcatcaagcggaaggacaaagtgtgacggtcaaactggagttcgataaagcgctgcaagcagcaagcgcagagctgggtggttccgccgtcacactgactaagacagcggatgcgaaagtctggaccggtgacgttgtcgtaccagtgtcgtcagagctaaccgtaggcttggtggtgaaagattaccaagacctttcggggaatacgggcgcagaagaccgcaaccactcgatgccgatcacaccaaccttggcgatcacgccagtaggcaatgtggatagcagcaatgccgccgcattgcagatcaccggcacttcaagccgctttgatggccaaaccgtgagcgtagaaatcaaagcgcaaggcagtgaaacggttatcgttagcggcagtgccacggtacaaagtggtggcgcttggacgagcaacgcgatggacatcagtggtgagccaaatggcacctacaccgtggtggtgacggggaccaacgcatcgaatgtcgaggccacagaaacgagcaccttcacgctatcgcaggcattgccaaccttgagtaacgcgacctttaacccaacgcaccaagcggaaggacaaagtgtgacggtcacactggagtttgataaagcgctgcaagcagcaagcgcagagctgggtggttccgccgtcacactgactaagacagcggatgcgaaagtctggaccggtgatgttgtcgtgccagtgtcgtcagagctaaccgtaggcttggtggtgaaagactaccaagacctttcggggaatacgggcgcagaagaccgcagccactcgatgccgatcacaccaaccttggcgatcacgccagtaggcaatgtggatagcagcaatgccgccgcattgcagatcaccggcacttcaagccgctttgatggccaaaccgtgagcgtagaaatcaaagcgcaaggcagtgaaacggttatcgctagcggcagtgccacggtacaaagtggtcgcgcttggacgagcaacgcgatggacatcagtggtgagccaaatggcacctacaccgtggtggtgacggggaccaatgcctcaaacgtggaagcaacagaaagcacaaacttcacgttggcacaggcgttgcctacactgaccaacgcaacattcaatccgacgcatcaagcggaaggacaaagtgtcgcggtcagactggagttcgataaagcgctgcaagccgcaagcgcagagcttggtggttccgccgtcacactgaccaagacagcggatgcgaaagtctggaccggtgatgttgtcgtgccagtgtcgtcagagctaaccgtaggcttggtggtgaaagactaccaagatctttcggggaatacgggcgcagaagaccgcagccactcgatgccgatcacaccaaccttggcgatcatgccagtaggcactgtggatagcagtaacgccgccgcattgcagatcaccggtacttcaagccgctttgatggccaaaccgtgagtttagaaatcaaagcgcaaggcagtgaaacggttatcgttagcggcagtgccacggtacaaagtggtggcgcttggacgagcaacgcgatggacatcagtggtgagccaaatggcacctacaccgtggtggtgacggggaccaatgcctcaaacgtggaagcaacagaaagcacaaacttcacgttggcacaggcgttgcctacactgaccaacgcaacattcaatccgacgcatcaagcggaaggacaaagtgtgacggtcacactggagttcgataaagcgctgcaagccgcaagcgcagagctgggtggttccgccgtcacactgaccaagacagcggatgcgaaagtctggaccggtgatgttgtcgtgccagtgtcgtcagagctaaccgtgggcttggtggtgaaagactaccaagatctttcggggaatacgggcgcagaagaccgcagccactcgatgccgatcacaccaaccttggcgatcacgccagtaggcaatgcggatagcagtaacgccgccgcattgcaaatcactggcacttcaagccgctttgatggccaaaccgtgagcgtagaaatcaaagcgcaaggcagtgaaacggttatcgctagcggcagtgccacggtacaaagtggtggcgcttggacgagcaacgcgatggacatcagtggtgagccaaatggcacctacaccgtggtggtgacggggaccaacgcatcgaatgtcgaggccacagaaccgagcaccttcacgctattgcaggcattgccaaccttgagtaacgcgacctttaacccaacgcaccaagcggaaggacaaagtgtgacggtcacactggagtttgataaagcgctgcaagcagcaagcgcagagctgggtggttccgccgtcacactgaccaagacagcggatgcgaaagtctggaccggtgatgttgtcgtgccagtgtcgtcagagctaaccgtaggcttggtggtgaaagattaccaagacctttcggggaatacgggcgcagaagaccgcagccactcgatgccgatcacaccaaccttggcgatcacgccagtaggcaatgtggatagcagtaacgccgccgcattgcagatcaccggtacttcaagccgctttgatggccaaaccgtgagcgtagaaatcaaagcgcaaggcagtgaaacggttatcgccagcggcagtgccacggtacaaagtggtggcgcttggacgagcaacgcgatggacatcagtggtgagccaaatggcacctacaccgtggtggtggcggggaccaacgcatcgaatgtcgaggccacagaaacgagcaccttcatgctatcgcaagcattgccaaccttgagcgacgcgacctttaacccaacgcaccaagcggaaggacagagcgtgaccgtcagactggagttcgacaaagaactccaagcggtaagcgcggcgttgggcggttcggcgctggcattaacccaaacggctgacgcgagtgtctggaccggtgatgctgttgtgccagtaacgtcagagctgaccgttggattggtggtgaaagactaccaagacttgtcaggcaacacaggcgcagaagatagcacgtattcgatgccaattaccccaacgatcacgatcggaaccatcagtgatgtatcgggtaatacaacagtaactatcaatggcacaactactcgttttgaggcgggggagataatagcactcaaagctgttgataccgatagcttggtggttctaggcagtgcaacagtgttagttgatggtacatggaccgttgatcttgatttgtctactttgaaagacggagtaataaccgtttatgcaaatggtgccaattcactctttgcgaccgctgatgaggtgaatacaacattcaattactctagtactacagcgcttgttgttccaagctattgggaaagatattccgcggagttaccttcccagaaagcagcctaaaggaaagcgagcattaagctcgctttctttttgcttgtatttaagtgatctagttcaaatgattatgcttttttcataaatgaatcagataactttatcgccttaatacaaggtgaaacatctagaatgcctagaccatttttgtttttacgaggcaatactgtgattgtgatggtaattgatgatcttcaatggtgaatatgccgtgttgataatatctattaatattatatagttacatgcacgtctataccctaggtgtagagttctgcattaggtagtgattactacctgagttgtgtatctatgatgaagtgaaattagcctagcatcgcttcatattagttacacctttgtgggtgtgacacgcactcacctttttattcatacttcctcagtcgcgataggttatttattgctactgaggaaggcgtatccctccagaacctcaaccttcggctatcagtgcttaaattgaatttgttaacttctgcccaaaagtaaaaaactgtttttgcttaaagcctgccttttgagttggattgaagggtaataagtttgaattgatagttaaaacaaccaaataacctgtggttttgttatttctcgggcatttcggttttctggaagttttctatgtgattgaatgttatgaattaaattagtagcattagcacaccaaatcgtacttgtaagccttatcagcttaaacgtcaccgcaaccatgtttacgatgttggcttggaagcagtataacgaatctcaacgcaagaaatggctgaaagattggattgagaaacaacctgcggtaactcaagcacccaagcccttggaaaagtcacttgataacccttcctagatgcaaagaaaaaccgccttcgggcggtttttcttttcttatgacttcatactgaatttagaattttctttttaagccccatcaacctgccgcagaaatgtcgtatttcttcatcttatggttgagagtgctcattggcagcgccaattgctcagcggccttcttggtgtgccaattacacgcatttaagcaatcgatgatcactgtacgctcgtattggctgacaacatccttaaggctctttgaggcgtcttctaaatccgcttgtgcctgccgctcgtaagcaggttggtctggggtaggtgaggctgctttcatgtcgatatttggcacatcttctgggatttgttcgccaaactcaatgtgagtaatggtttcaaaatcggacagtagcaccgagcgctcaatgatgttcttaagctcacgcacgttccctgggtattggtaggcaaggagctgtttacgtacgttggcacttaaccctggcgcttgcggcaaacctagcgtgttggttgtgtgttgcacgaaatgttcggcaaggggaataatgtctgacttgcgagctctcaatggtggtaatgtaatagggaacacattcaaacgatagaacaaatcggcacggaatccgccatctttgatttgctgcataagattacagtgggttgcagcgataacgcgaacgtcgacttcaatctctttgctggtaccgatagggcgaactttacgctcttgcagtacacgcagtaacttcgcttgtagtagcattggcatatcgccaatttcatctaggaaaagcgtgccgccgttggctgcttcaaacaaaccgactttgtctttgtccgcgcccgtgaatgaacctttcttatgcccaaacagttcagactctaacaactgttcgggaattgccgcacagttttgtacgatcaaaggctgttctgaacggttggagttttcgtgaatgtatttcgcgatcacctctttaccagcgcccgtttcaccacgcaacagaacgtctacaggaagagatagcaccttatcaagacgattcaagacattgagcatttcttcgctctctgcaataggaccttgataggctttctgagtacgttttttgagctgagtgttctcgcgcactagggcttgattatcggcactgaggcttttaatcacttgcccatattgctctaaccaaacggcttggcgaatattactcgcagccatacgacaaaactcagtcaaggctgcctcgttatcgatgacgttcaaatcgagtaagactaacagcccaatggttttgctttcgctatcaatcaaaggccaagccagtaagttctcgctttttagtcccaatgtctgctctgtttgataaatgctttcgcagtcgtagcctttgtatttgtataactcgttaatcaaaaccacttcaccattacgaatggcaaagttaaatgggtcgttctcactagcggaatcaatctgcaatgcctcccaagggtgggaaacaatcgttttatcgttatggtgagcggtacttggaatcaacgcctgtcctgtctggtccaaaacataaatgatgccgtgctttgctaaggtcatttgtctcgcagcggtcaacacggcatccaatgtttgggtaagctggctgcgatcagccaaagattggctgatcgcaagtagggcgttgctaagttcgctatggttagctgaacgcataggtcagtgttccttgctcatcgacagaaactgtgatttgcgtgtgtgaatcatccttatcatgcaccaatagctgtgtcgatagctgaggtagtaactgacggttgatgaccgcgtcaatgttacgtgcgcctgtttcagcaaggcgacagttacctagtacgaactcgaccaagctttcttcgtagctcagggacaacttgtgatggctatgtaggcgctgagaaactttgtttagtttgtggtggataatttctgtcattgcttcgtcagacagaggcacaaacggaagtacggacatacgtgccaacagcgccggcttgaagtgttggttcaaagtagggcggatggcttctgcgatgatgttagcatcaatttctttagactggtgaaccagagattcaatttcgtgcgtcgcaaggttgctggtcatgatgatcagcgtgttcttgaagtcgatggtgcggccttcgccgtcgttcagcgtacctttgtcgaacacttggtagaacaggttcaacacttctggatcggctttttctacttcatcaagaagtacaacagaatatggacgctgacgtaccgcttctgtcagcatgccgccttcaccgtaacctacgtaaccaggaggcgaaccgattaggcgagataccgtgtgcttctcttggaactcagacatgttaatggtcgtcataaagcgctcaccaccaaacatttgatcagcgatcgcacgagcagtttcggttttacccacgccacttgggccaactagcaggaatacgcctgtcggtgcgtctggattacccaagccagcttttgcggtttgaatgccttcagacagtgcatcgatagcgtattcttggcctttgatgctttgtgttaggctttctttcagttttagcgtggtttctgcttcgtcttgaagcatcttacccattggaatgccagtccagtctgagataacgtggctgacttcatctggcccaacttcaaagtgaactaatgggttgccattgcggattgcgtctaactgttcttggcatgctgaaatcgcaatgcggaccgcttcttcatccatttctgtatatggagaaacgtcttgttctgaatcttcgtctacaacaagctcttcttcatttactttgccaaagaccagttcatgtaagcgagagcgtagagcaatcatctcttggatctgctctttttctttgtgccattgcacttcttgttctgctagctcttcttttgtcgtttccattgccaattttaggtctggaatgttagcaaggctgtgcttgtcgccagtttgctgcagagcatcacgctcaagcgcttcaagctcgcgctgttgagctgcaagttcttgctgaagagtctcaacagaagcaggaatggcattaaggctgatgttcactcgtgcacatgccgtatctagtacatcgatcgctttgtctggcagttggcgaccagagatgtatcgagcagaaagcgctgccgctgcggtgatagcgtcgtcacgtacgtaaacattgtgagatttctcgtacgccgggcgcagaccacgaatgatcaatgctgcttgctcaggagacggttcgtcgagtttcaccagttggaaacgacgcgcaagagctggatctttctcaaagtactttttgtattccgaccatgttgttgctgcgattgtctttacttcaccacgtgctagggcaggttttaataagttagcagcatcgctaccgcccgcttggttgccgccgccaacaagggtgtgcgcttcatcgatgaacaagatgatcggtgttggcgagtttttcacttcatccaatactgcatttaggcgtttttcgaattcgccttttacgcttgcgccagcttgcagaagacccatgtccaagccgtaaagctcaacacctttaaggttgtctggtacgtcaccttgtacaatcttaagtgccaagccttctactactgcggttttaccaacgcccggctcaccaacggcaatagggttgtttttacggcgacgagcaagaatgtctacaatttggcggatttcttggtcacggcagaaaacaggatcgatttcgcctttgcgtgctttaccagtgaagtctgtcgtgaacttgctcagtgctgaaccatcttcacgagcttgtgttttctctgaagtcgcgacttgcgcctcaatagagtggcttgttagttctgcaaagttacgtttaaggctatcagggtttactgcttcaagaatagaggcgtaaccatgttggccataacgcagagggttgctaacaagagtaaggagtagggcgccagagcggatttgtgtttcggatagatctaaagaagaaaccagccagctttcttgtaaccattcgattaataaagcagaaaaaacaggtttgctgccatttcctttggcgttggtatcgagtgtggatcgaaccgattgtctcaataagttctcagaacaatcaaaatgacttaataagacgtcaaaatcactatttggcctttctaatagactcagtaaataatgctcaatttgaacttcgtttgctttttccgagactgcgagtgctgcggcatcttctaatgctactttagctatcgggtgtagacgctgaattaatgaagaaaggtttatatttatcatagccattttattctttagaggaaaagaggtgagagctattatactaaaagtggttacgccatttttttatcaagatattttaactatcgttttctggtgatttgaatggctatgttagtgggaatatttatcacaagtaattatgctttgctattgtgaattaattattggttaatacgactaaaatgccagtgttattaactttattttgttctttgtatatggttattttaactttttaaaattgaatcaatttattattatctgattccaatttattggttttttatccaataaattgtacgaaataaattaacctataatggggttttattgtaactcattgtttttgctctactttaaaagttggcacggagtttgattataccaagtagtcgcggtgataagcgatttggtgatttaaacaaaacagattttagaaaaggaaatagcgatgccaactccagcatatatgtctatcaacggtgaaactcaaggtcacattactaaagatacatactctgcagattcagtaggtaacacatggcaagaagctcacgttgatgagttcctagtacaagaacttgatcacgtgctaactgttccacgtgatccacaaagtggtcaacctactggtcaacgtgtacaccgtccacttgttgtgactaaagttcaagaccgttcttctccactactatttaacgcactagtgtctggtgaaaaacttcctgagtgtttgattcgtttctaccgtacttcagttcaaggcaagcaagagcactactactcaatcaagctgattgatgcgctattggtagatatccaaactcgtatgaaccactgtcaagatgcagcaacagctgatcgcgttacagaagaagttcttaagtttacttaccgtgcaatcgaagtaactcacgaaaactgtggtacagctggtaacgacgactggcgtgctccacgcgaagcataattgctttttgcgtaaagattcagggccaacaattgttggccctaattcgattatttgtatcaggtaaaagatatggtgaatgacgtagaatttaaatttgaagtgccagggtgtgggcacgagtttcgggtagaaagctttcaagtaaatgaagagctttcgaaaccttttcacatcagtctctcattactttcacttgatccagatatctcttttgattcgttaatacgaaaagcaggtacactgactctttatggacaaggcttaagcgcggcacgcatattcaatggtgtggtgaacgaagttcgttatttgggtacaggccgtcgcttctctcgctatcagttaattttggttccgcaggcttggtttttatcacagcgccaagactgtcgtattttccaacaaaaatcagcgaaagatataattacagaggtgcttgatgatggttcagtgaccgactatcgctttgaattgtctggtatttatccaccaaaggagtacgcactgcaataccgtgagtcagacctacactttgttcagcgtatgatggcagaacacggtatgtggtattatttcgaccacaccgattcaaaccacacaatgatcattgtcgatagtaatgatgccattgctccactggttagctctcctctcaatgcatcttatattgggccgatcgtatatcatgccgatagtgggggtgttgctgaccgtgagcatatctccgatctcgagttagttaaccgtgtgagaacgggtcaggttacttacactgactacaactacgagcagccaaagatcccgcaagagatgactcacgcaggtgatctggaccaagatttaaagcagtttgattatcctggacgttatgttgaccctgtaatggggcaggtgagaaccactgagtggatgtttgagcacattgtcgataatcagcaagtagaagcgagcagtgatgtgatgcgtttggcgtctggttacagctttaacatcagtgaccatcctcgctctgaaatcaaccgtgattacatcatgttatccgtcatgcacacagggcaagacccacaagtgcacgaagacgaagcgagtggcatgccaactacttactacaaccagttcacgtgtattccacgtgatgttgtattcaaagcgcccaagttggcggctccagtagtggatggtccgcaaactgcggttgtagttggtcctgcaggggaggagatttacaccgataaactaggacgaattaaggttcagttccattgggatcgctacggtaacaacgatgaacatgcaagttgctggattcgtgtgagtcaatcaatggctgctccaacgtggggtgcggtttacttgccacgtattggtcatgaagtcgtagtcaccttccttgaaggcgacccagatcgcccattggtgactggcgcagtttataacggtcttcacttccctccgtactcgttgcctgagaacaaaactcgtactacgttccgtacgcaaacgcacaaaggcaccggctacaacgaactgagttttgaagacgaagcgaaccaagaagaagtctacattcatgctcaaaaagacatgtcgaccaaagttctcaacaatcgttaccgagatatcggtcaggatgagttcttaaaggttgcgcgtcatcagacgaacgaagtccatggtgatcataaagaaaccattgatggccacaaaacaacccaagtaaacagcacttttaccgagacagtagagcaagacgttaccgttacatacaacgccaacgaaacccaatatgtgaagaacaattctgatttggaaattggagataaccgcacaaccaagatcggcaagaacgatgatttggatgtgggtgaaaacagtaacttaactgttggtgcatcaaaaagctcggacattggtgccgatgataaccaaacagtgggtggcaacttaaccgtatcggtaaaaggcaatacatcttacaaagccgatggcgcgacgcagatcatcagtggtgacaagattgtactgaaaacaggcggttcttctttagtgatgaacagtgatggttcaatcaagctctcagggtcttcaatcaccattgaaggcagcgataaagtcgtcgtgaaaggtggtaacgtagcgattaactaatgatggaaaaacaaactcttattaaagaaaccatcacgaaacagcaagccgatacaccggcttgcttttcgcgttttggaaccatagtcgccattaacgaatcaacaggcagtgttcgcgttgattttgatggcaacccatttaaccaaccacttaccgcaaggttagggcgtggctttcgccgctctgaattgcaaatggctatcgacaatcgattaaattgtcgaatcgaatttttgaacgacgacttaagcttgccccttgtgacggacatttttttctccattctggatgactctgatgagtttgtattgcgggcgaagaagatggtcattgagacggagcaagaactgatcgtcaaaagtggtgagacagaaactcgctacagcgggcgtgatggccgtattacaactaaagcgaagtacgttacttctcaagcagaaaaagcacaaaaaattcaaggcggaacgatcgcaattaactgatgactatcagtgttaactaattacatcgtgttaactaattgctatcgcgcgctaacaacggataagtagatgcgacaaagaattggtgtcgagcacctaaaggcacccataaccaatcaagaagtcgaaggtgcgttggccaaagctgaacgtgctgttaaagacctttcccaattgccagatacatggctggacttctgcaacgaaaagctggctatcacttctgagagcctcggttttttgatcaggcagcgtgttcaacttcataaacgtggttatccgagccgtgaactggattacctgaaattgatcgaacgccaaattgaagagctcaagaaagtgtacttgtcattctatcgattagctccaggtctgattcatcaattaagaggtcgagaaccagaaatctatgcttggctcacgctacaaaaagagctcggttctgagttcgataacctcttgtgtggattaagtttactcgaagagttagacgctcaaaccgcgatggtcattgttacgcaatcgccagttgaaaacatggatagcgtgatgtcggagctcattgagggcaaagcgaaatccagtacgttttactttgaatacttacgtgtccgccaggcattgagtgttgcgttgattaaacgttggcacaaagccgaaattattcgttctaatatcgcgctacctctactggcattgcaagatgtggaagaggggattgaatggattaatgacaatgcaaatactgaacaatatctgtttgaacgtttgatcactaaacgcgacagaggcacatggttccgtcagtgttttggtatcgagccgagcggtttgccttctgcacaagtgttgacctttgcaaagctattggagatcaaagagtttgaagcgtttgatatatcctcttcattagctcccgttgattttgctctgagtggcgattggaagttagtgccagaaatcatcgagcacttggaaacactggaagaggatgagggcgaaatttggattcaagctttgtatgtggtttacggaaagcttcttcccctgactcctcaaggtgttggtgtcgagtatgaatgggaagagatcgtcgatctgctcaatgagtgggtggaagatgaaaaatatatccaaaacctaccgagccgcttgggctacgcattgagcttcgaatctactttagcagcgatgcaagatgccaacgtcgatgtcttgtttcgagattggctatggcgacaaatttgcattcaatctcgagcctacgtgccttgggacatggcgatgccagtttaccaacaagattggaatttcaataacctaaaggccgcaccttctgcgagtgaacgattcaatttaaggaatagcaatgcagttatgggatattgaagcgtacccagagctatcgattaaaggccgttttcaacgtgacgaaaacggtgatgaagtttgggttgtggttgcgaaacgcacatggcaattcgacggtgaggtttggcacgaattgggtgacagcgaaatcttcgacgatccccagtatttgggcgaagaaggtttctctgcgatcaaggtcgaccaagagttcgcttataccaaaaacaatactgatgtgcttgtctacggcaaagctcgcagctacgcgaaaaagccagtgacataccaagaatgtcgtgtgctgattgatggtcatatcgataaaactctcgccgttcacggtgaacgcgtatgggttgagcatggcggcagcgtcacattgagcaaacctgtgccttttattgaaaaggatatcgattacacctgcgcaattggtggtgatttacgtaaccgaattggcggtggtgtggcggattcgaacaaagaactgcttgagcagaaagtgccttctgtgttctatccaaaagaagaatggagctctacatcgaaaaatctaagagtggctggctttggacctgttcctccattctttgaggctcgccaaacattggctggtacgtttgacgaagaatggattgagaaccgcaagcctatgcttccattggattttgatcgacgtttctttcaaagtgcgcctgctgatcaacaatgcaaaggctttcttaaaggtggggagcgcttgatgatgagtggtttctgtcatgatgacacgatttcattccgcattcccaaagaaaagtaccgcgcaatcgcgacgttcaaagacggcagttatccagttgacatggcgatgtacactttatttgttgatgctgaaaagaaaaccatatcaattagttacacagctgcatttccttgccagagcaaagagcatttattggtgtcgacgtctattacgaaagtggaagaggtaagcgaacatgcataactcaacaggtcgttacgtgctgggaatggaagtgatgacgcctactggcatgaacttggatattgctaccagtgtagcaaatgctgacttagccagattcgaccaagtagttggcgaggacggaattgagcgtttcacctttgccaaaattgaatacacaaaagaaagtgatttgttcgaaaaaacctgtgagttaacggcaaatttattggattcgcttttgacacagctgcctcgttctttgaagccgattccattacttattgctgtgcctacgacaatttcattagtgaagatccaagaatggcttggtgaaagtgactattctgactttctttctgtggtcgaagcagttcacgatagtggccctagcttcgtgctacaagccatgaagtcgttggataaatacgatgccatgatgtgtattagcgtcgattctatggtcaacagaatacaagaactcatcgacgataccatggtaatgagcaccaataatccatggggtgttattccaagtgagggtggcgcgggcctcattttttgccgaagaaataccgtagaaacactcaaacttaagcctttagcgcaacttggttatatcgatacagaactgaacacttcagaccgaagaggtatgtatcgtcttgtacagcgtgcgagtaaaaagcttactgctttcggtgaagtgtattctgatatgacgaacctgcgcgcgcacagtgaagattacggtttcgcgctgggtgccaaagctgagcgatttattaacccagaacaacctttgcttatcaacgagttatgggggacgatgggaaactgttcatctcttgctcttggagcatttgcggttaaatatcatcactttaaccagcccgtaacgctgctgatgtttgacttcgggggtgataaagcgctacttcaattactagcgtgctaatcattctttgtcgaaatatataatgaaaatgaagtcatattacgttgttgaaattttaatttctaaaacaacgtaattcaggtagtgaaagcgtaagtcaaagagtctaataacttagtaattataactaaaataataggcttactgttcgttttaaatactacccttgaaacaaggatgtttcagcaaatattatttcggtctaataatatttgttccataaaagagtgcaaagtgtctgatattcagggtgcagccaaaggatacattgatcagttggttgttaaagccattgagaaaaagaaactgaacgagcaaagattggcgcagaaaaataagccaaagcttgagtacgttcttttttcacaatttgatgtattagacacgttgccatctaaaaatggtagaacgacggtttaccacctcgcaaaaaaaggccaaccagaaaaacaaatttgctgcaaagtcgtcaatgaagacgccccagaagtcgcaaacaaaatgttggcgaatgaggcaagcagattagaactttctcagcatccaagtgttgcggactttattaagttcggcaacgagtttgaacgaccatatttaatgtatgagtggattcaaggcgaatcactagcggaaaaaatggaacgctattcttccaaaggctttcgtcatgatcatattgcatggttagtttatcaactcgcaggtgcgctagaatttatgcacactcgaggtatttgccatcttgatatcaaaccgtcgaatgttctggttagtgaaggcgactacgtcaagcttatcgactttggtgcagctcgttatatcggcgagattgaatctcacgcggaagcgagtttaagttacgcgtcgccactttatctcgagacgggaacaactgagccacaagacgacgtttattctcttgcattgcttagcggtcacttatttttaggggcgatttttggtgatgcttggcacaagcaattaaaagatcgcaaacgtcctgcattaattccgaaccatatttggaagttgctcaagcaagtgataaataacccaagagggcatggctacacagctatttcttttgctcagcaacttgcccgtattgatactcaatccatcgactctcattctaatgcgcccatttttagtagtttgcgtaatgcggatctactgctgactcattgtagacctcaagacaaagcgggctatggccgctttaaagtgcttgaagcgagcttggtattgagtgttatagctattacgggtaattacctttatcaacaaagccagccagaatggaaagcgattgtaaaatcagaacctacgatttctaaaaccgaaacaccgttggtgagtaatatcaaaccagcgcaaacggcgtcttttttggctcaacctccttggattattgaaaaggctcttaatgatatggaaaaagacgttgtgtcgacagcaccatatcgagaagcttatcaagtacagcaaatgaaactatctgcgctgtataaacaaaaccaagcacaattaatgtcttaccaagctttggcagatgacttgccaaaagagttggtggatttacgcaaagagctagtcttattacgagaaaaactcgttgatgatggtgcgttgtttccatacgcagataagactctgactcaagtaatggcagggttaaacacagcgtcagtaaactcgatgaaaatctccgctgttgctggaagcaaagatgaacgaatcgccaatttgatcttggatggtgaagcgaagttggctgatgaagagatgaaagcggcttggttattaaatcaatcggaggcttacttttacagccaagtgctgccaaacaaggtgatggcgaacatcaacgactctattgaacagcatgcaaaagatcattactacaccaaagccatagaagaggcgaaagccgctaaaacgtactttggaaatgcgccagaattaaacaaaaaggtgaaagacctgatcgttgctcgcagcgaatacattctgtttagcactgtgacggagcaatcggtgtttgataaacaaaaacttcacgattctctggctgaccttgagcgaaatgcgccgacaaagtttggtgaaattactgatctgcttgaaagcatggccacagactctatcgataaaagtcatcaactttctcgtcctgcacgtggtgcaattgcaattgaacgcgccttgagagattacctagcagaggagagaagttaatgtctgtgattgaaattgaacgtttacttgcgcctatctctgacgtaaatcctgtcggtgaagatgctcgttacgagttttgttatgaaatgatggaatccgaagtgaagaagttcggatccttgtttggcgaaacggtagattggagtgtcgtaaaaacgcacgccactgaagttcttgaacaccacagcaaagatttaaaagcaatttgttaccttgttcgcgcgcttacagaagagtttggtctgcaaggtttcgagcaaggtctaaagctgcttagtgaagcgttaaatcgctttggcgttgagctgtacccgtcgcggaaacggggtagagatggtgcggtagagtggttgaaccatcagtttaaattggtgagcagcaggtttgcagaaagcgctcagtcatgggatttggtttcaggttgtatttcgactatcgaagaagtccaacgtcagtacgacgatatttaccaagactccgaagcggacttctttgaaattcgtagtcaactcaatgcgctatctcaacaagcggctgtcgatgagcaaagcgatggtgcaatggctaccatcgaacaaacagctccgctaacggcttcacagcctgtagcagtagaaactccggcacaaagtgaagttcaaaccgttgtagcaaaagcggttccgtcggctcctaaacctgcaccagttaagaaaacagtaacaaaagaagtcgatgtcgatacggatttttcatcgccgaccgcttctaaacgtacacttaagaaagttgcggaagttatgattcatgcgaatccaagtgagccgctagcttaccgaatttatcgtcacctaacttgggatgatattgatggtcttcctgatcatcaaagcaatgaaacgccattgagtttggctgtgtcgtctgatcagcaggctgagtaccgcgataaagcctctcaagaaagcgacattgatacagttaagcgtttggagcgcacgttaaccgatgctccgttttggctgacgggtcactactttgtttattcgatgttaaacaatctaggctttaatgacgcagcgttcgctgtgaagcaagaagtgaagcgttttgttgattcattagagggcattgaactcctcacatttaagaattctataccatttgcagatgaagcaacgttgagctggttatcgactcaagctgcaacttcttcaagtgctcaatctgtcgttcaaacgattgtcatcacagatgaagactcactgccaatggaagatatcaccctagccaacttaggggagtatgccgcagaactcgcacacaaactcgaactggatttctcgggacgtgggcagtttatgctgcatttacaattgatagcagcttaccagtctgttggattatacccgttgtgtttgccttaccttgaaaaagcttgggaagtgcaacaagcgtttaatctcgcgagctgggaaccccacttatcgtcacagttagaagacctgattcgaaaaacactgcatcagttattcagaaacaaggatcttttgcctgaacagtacgaaaagtggaaagcaatttatgactaattaaagagttagataaggaactaattatgtcacgtgacggctcggtggctcctaaagagcgaattaatatccgttatgttccagcaactggtgatacgcaagaagacgttgagctgccgctgagcatgatggtggttggtgactttactgcacgtgcagatgagacgccaatcgaagaacgtaccccaattaacatcgataaagacaactttaatgacgtgttagaaggcatgtctccaaacgttaaagtaaatgttgaaaaccgcttatctgatgaagaaggcgcacagatcggtgttgacctaacgttccaaaatatgaaggacttctctccggaagcgatcgcgaaaagtgtacctgaacttaacagtcttcttgagcttcgcgaagcattggttgcactaaaaggtcctctaggtaacgtacctgcgttccgtaagaagattgcatcggttcttcaagacgaagaagcaagaaagaaactgttggatgaactaagcattggcaacgaccaagacgcgaaagaagagtaagggatatcatgtctgcagaagcacaagctccagaacaagaagcagccttagttgaatcaggctcacttctggatagcattcttaacgaaactcgtttaaagccaagtgatgaaggttttgatgttgctaaacgtggcgtagaagcattcattagtgagctactgagcagctcgaatacagagaaagtagaccaatctttggttgacttaatgatctctgaaatcgaccaaaaactgtctaagcaagttgatgcgattcttcacaacgaagaagttcaagcgattgagtcaacttggcgtggcctgaagtatcttgttgatcacactgatttccgtgagaacattcaaatcgaactaatttcagcgaagaaagacgaagttcttgatgattttgaagatgcaccggaagtggttaagtctggcctttacaagcagatctacactcgcgaatacggccaattcggtggtaaacctgtaggtgcggtaatctgtgactacaacatgtcagcgtcttctccagacattaagctgatggaatacatggcaaatgtgggcgcgatgtctcatgcacctttcataacgtcagcatcggctaagttctttggtctggatagctacgaagagctaccaaacctaaaagatttgaagtctgttttcgaaggtccacaatacacgaaatggcgtggcttacgtgaacacgaagatgctcgttacctaggtctatgtacgtctcgctttatgttacgtacaccttactctgtcgaagataacccaattaaggcgtttgactacgacgagcacgtaacagatagccatgataactacctatggggtaactcagcgtacgcgatggcttctaagatcagtgagtcatttgctaagtaccgttggtgtccaaatatcattggtcctcaaagtggcggttcggttcatgacttgccagtttataactttgaagcaatgggccaaatcgaaacgaagatcccaacagaaatcttggtatctgaccgtcgtgaatacgagctagccgaagagggctttatcgctctaacgatgcgcaaaggttctgacaacgcggcgttcttctctgcaaactctgttcaaaaaccaaaagtgtttgcaaacacgccagaaggcaaacaagcagagatgaactacaagctgggtactcagcttccatacatgttcattatcaaccgtctagcgcactacatcaaagtgctacaacgtgagcaaatcggttcatggaaagagcgttctgacctagaaatcgaattgaacaagtggatccgtcaatacgtttctgaccaagagaacccaccagcagaagtccgtggtcgtcgtccactgcgtgctgctaaggttgaggtatcagacgtagaaggcgatccaggctggtacaaagtatcaatgtctgtacgtcctcacttcaagtacatgggcgcaagtttcgacttgtctctagttggtaaactagaccaataacccattattgaaaccatcaagcagccattcttcactggctgcttttttcgtaggaccgaacatggaaaaaggttatcgtcttttagagcggattgaactaggtgagccaaaaaactgctacgagaaagtggtctcccacaagcatttgattgaatctattcatttgcatcttgcagatttgctgaatacgcattctggtaatgcgatgatcgatagtgaatatggtttgcccgattttaatgacgttctgtctaataatacgaatctcgttcgccacatccaaaagaacatcacagcgaccatagaacgctttgaacctcggctacttaacgtcgaagttcactatcgtgaagaccaccacaatccattacagctcggttttggtattcgcggagaagtttcccataacggcggtaaagttccaatgtcgatcgatgtctacatgggaaccgacggccaattcaacgtttagtaggtgtcacttgagtaatagcaaatactttcaagatgaactcacatatttgcgcgaatcgggtagtgaatttgccaaatatcatccgaagttaactcactttctctctgaaggaacattcgacccagatgtagagcgattgttggaaggtttcgcttttctgactggacgtattcgagagaagatcgatgacgaactgccagaactgacgcaatctctgatgactttgctgtggccgcattacatgcgttcaattccatctttgtgcatcagtgagctaaaaccccatacgggaagtgttactgagaaaacggtggtaaaacgtggcgcagaaatggccagtgagcaagtggagggcacgcaatgtcttttccgcacttgctacgatgtgaatctgtacccaatcacgatcaccaacattgaacaaaccaacagccgcaccagttcagcgattgatgtgacgttgtcgacggagcatggcctagagctgtctcgaattggtttagatacgttgcgtttgcacttgcatggtgagattcatattacccgcacagtcttcctctggctgttccgttacttggattacgtggagttggatgtcggtggcggctataaacatcgtttagggccagaatatgttaagccggtagggcatgaagaagacgaagcgcttctgccttacagcaaaaattcattcgcgggttatcgtttgctgcaagaattattctctttgccagacaagtttatgttcttcgacattacaggtttggagtggctaaagggtattccacaacgaagcacagtgaaaatcaaatttcactttaaacgcgcattgccgtcagaggttgtacttaaagataagcacctaaggttgcactgcacgccggcggtgaatttgtttgaaaaagatggcgacccaattcgtttggagcaccgtcgtaacgaatacaaagtccgtcctcaaagtaatacacaagagcactatgaagtgtattccattgagcaagttgagagttggagcaaagacgagcgtcgccgtaaaccgttaatcgaattcgaatcgttcgagcatcaaatcaaccaacgagacaagcgagaattctacaagtcgaaggtaggagagcgtgttagtggaagaggcttagagcgctatatctcgttccacactcataatggtgatattgcggatttaggtacggaaaccgtactcatgaaactgcaatgcagtaacgctgatttggccgaacgtctttcggtcggcgacattacttacgcgacccacaaatcgccaacgtatgcgacatttaaaaacatcaccaagccaacacaatcggtaagcccacaggtaaacggcgagcttcaatggcagttgattgccaacatgtcgctcaattatttgtctttggcaaacattgatgtattaaaagtgctgctctcgacctatgactttcattctcgtgttgacagacaagctcaccgagcatcgattcaccgactcgatggcattgtatcgtcagagatgaaaccaattgaccgtgtattccgtggcgtgtctgtgcgtggtaaccagttcaaactagtaaccaactctaagttcttcgtaaatgaaggcgatatgttcctgatggcaacggtactgaacgaattcatccgtttgtattcgagtgtaaactcctttacggagctagaagtatttgatgaggcaaccggcgaagtatacaactggaaaagcttaatcgggcagcagacgattctatgattaatacactttctgaaaaatcgaatgagtttagtttttatcaagcggttcttttgctcgagaagcactaccagtttgaaccaaactctaactttgtcgccgtgggagagaacaaatatttccaccaggagcgaattgagttcagcgtgtcgcctaatctatgcttccctaagagtgatatggaatttatcgttcatatggaacgaaatgggcagcagtattcacgagtcgagacgaacttccttggtttgcatggttcgagctctccattgcctgtctcttatacggagaaacttactgggcgtgaacctgatgacaacccggtaaaagatttctttgatttcttccataatcgctacaccagtttactctatcgtgtctggaagaagtaccgttatcatgtacagtaccaaagtggtgcgacagacgctttttctggacggatgctgcacttagctggtttatcaggtgttatgcaagattgtggtgttgcagagttagaccgtgcaaaagtcctgtcttacgttaatcaactgtcgacacgtacacgttctcctaagctaatttcaggtatcgtttcacattatttctcacttcctagcgttcgcatcgaagaatgggtttatcgccgtgtggagattgcagacagtcaacgaaataaactcaatcgagccaattgcatattaggtcaaagctttcatttagggcagagtatcgcagatttaaatggtaagtttaatctttgcattgatgacattgacttcgagacatttaagcagttttcttacgaaggtgagttgcataaaacgttggttggtttaatgcgattcatcttgcgtgacccgatgtcttgggatctcaaactgacagttaatctcgacagcattccaaataaccaacttggcaatggagaagggaacaacttagggcagacattttggttaggcaatcccacagatgaagatgtgaagatcagagtaattggtagcatttagatgctgtaggccaaaaagccgctcaatgagcggctttttgatgtgtgtgagattgggaaaactgtgttctgcctattctggttattatattgttatcaaatcgactctgggagcgtaaagttgtacaactcagccaaagtgaaagggtttttttgtgtcaagggagttatacggaggctcaaatccctccctgatacttttatgtcactaacgaagctaccgttatgagtgtttgtcagggtcgaatcacctagcaatctatatatcgcccattgaccactcttatttaaaacgtgttgtttaccttcgtctgtcacatcttggatcgtgatactaatgttgaagtcgccgttttgcggtggccactccatttcacgaattcggctcggaccgtgatagtagttgatatcagtgtcagagattttcaaactggctctgatagcacttgagtcaagatctaatactttcgcactgaatggaatataaactcggttagtactcttattgatcagcgtgtcgcggataacgttgtaattgcgcaattgcaccagtaaggctgaagataatggcattgtttcaccatccacctgttttggtacccacaagttggtatcgtagaacggtgcgaagtttttctgaataaatgtgtctaacaatccacctgaagcaaacaacagttcgaagtcttcgatagaaatctcctctgttgctgtgagatcaaatgggtatttatcaagtcctatctctttaaatttggtgtaaatttcactataccattgggtttgaatgccttttgctgattcggtgatcattaccgaccagctttgctttacgacatcaagtaaccaactgcgtactggctctggtgacttctgagcaatctgtttaagtttaattagcggatcggcttcagagctgctcattcggtgttgagcggctgcaagtgctgccatttgtggatcgggagcatccgcaatatctttcatgtaggtgcgaactcggcttaaagctgcgatagtttcgtcccatggagtaggggagttaggagtctcgcttacttgaagctgatttagaagatggaacgcttgttctacacgcttcatcaacaagtaatccggctgaaccacttcttcaacctcttctgacgcagagttcgccacttctgccagttttgggtctactttcgagatgagtacgtttttttctcctaccggtgaaaactgagtatttgcatagacctgctttaatacagtcgtcattggtgaagatggtccagagattaggtcgatagcatttgttagatcacccacattgctataactctgaatttttaactcactgagagcgttgcgccagtagttgatataatcatcggtgtactttttacgtacttgatttttaaacgattcttgctcttctttggttgggataacatagctggacaatcctagcacccagttatcactgatcacttgtttagacatcaagtcgacgcgagggcgatagaaggtactaaagccagtggaggtatagatcttatcgattactagtcggttgtcatctaacggctcgttaaatacgttgttaaattcaaagccaactgcacgttgtagatcgagtgtgccaagatcgatagtatctgcttggtttaaaatgcctgcgtaaacaagatccacatttgaatttgccaacaatgagcgacgagttgctcgaacgacatccatgttgatatctacaggtgagaattgagtgcggaagtaggcgtccaagtatcgcatagcttgatttaccacatccggcttatcgctcaagtctgtcagtgtttgcaatgtttggcgtcgtaaaaactgaatatctcgcttggtcggatccttcaacatcaagtagccttttaataacggcaacgttgctgtgacatcatgttggttgtgtgtcagttcaaaacggtagccttgctcaataacaggcataagttgttgagcaacctctttaatcaatgcttcatacgctaattttgtactttcgtttaacctgcttatatttagcggtattacttcttcatctaatgctcgtgatccttctaaccaagcattgtatgagggttggatagcacgagtagcactctgcaataactggttgaagcttgcttcttgttctgtttgaactttttcgtcgatattcagcaattggttaataacacgcaagttactgtctagtgttgtgacaaaaaaataagtaccgcctgccaataagacaacggatgccagcattgccaaatggctttgacgctgaatcatgcgtaggtaagtcttgttctcgcctgcgaagtcactctctgggaggatttgagagtcaagcaagaagcgagaaaagtacggtgtttcagttagttgcgtgtgctcagatgcgataattggtaaattgaagtaattactgcagcttttggctaacaagttgtacttgcgtccaccttgcaaagaagaacagaagaagatttcgcgaatatccaacgagtaaacgccattgtttgtatcgcaaagacgttcaagcacgtagctgatttcacgttgacagagttcaaactgttttgggaaggcaaggatggcttgcttttcgttgatatctgttgagtttgccgatgcgtctaaagcgttgctttctagcacttttaccagattatggaagctatctaggtagtattcggtaagtacacctttcgcttccttcaatggaatggacagaaattcaatacgtgtttttagtgaactagagtgaacgtattctttaaagccatctagtttgtcgagtttcgtcatcatcaaataaacaggtagtgcagacgaggtcttctcagtgatagagttcaaacgctgcaatagcgctgtcatgatgtaatctttctgctctggctcagatacaatcatgaattcgaaatcaacaaagaataagcaacctgaaaagggttttctcggacgatgacgaatcacctcatcaataaaagtattccagagagaagcatcactgcttgaaacttcctggaagatgaggcgttgatctggcttaacataaacggcaccatcagattcataccattcgaagaaatcgttttttgctgtgcgagaacggtccatcggcttaattgcgctggaattaaacaaaaactgacgtcgaccggcccctttattaccaaccatcagatagattggctttttgcccgcattagaaagaagagggcggatcaccattgaaagagcttcttcttccgtctctaaacgttgaacttttgagctgttttttttcttaagccatagggaaagctggaatgtaaaataaaaaccagtaataactcctaaaccaacccatagataaagcttatctgcgtcttcaagtatgaataaatataccgctgcaaatatcgcggctaataccaaagccactatggtagaaaccacgactggatttcttagtattttttgtttgaacataagcctgtaacttctttacttttatttattgtctttttctgcagcttgtagctggttgattttatataggtaagcgttcagcaaaaggagtgatcgttcaatttcctttctctgagagttcttttctgcatattcaattcgcccaagaaggggaaatagcgaattgctgtactcatatgctttcttagcgtccttgtctgcactttgttttgcaatgttggcaaagaccgttctagcatatgcaaaacgtttgtatatagcctcaaattcatcttcaaaataagaacgttggcctaaaaaattatcgcgaagttgttgtgcagaggatgagtctggatagagcttggtcaggtcattcgttaactgttcaatggtattgacctggctcactttgactggatctaatgtccaacctgacatcaaagtatgaagacgctcaaccgtgacagagcgcaagtcatcttggtaatttttgttttcgacactgagcgagttaacaatcgtcactaattcagtaagtgatgctgacccaattctttccttgagtattgtgtattgatgctgtttgtatatgaagtgagacgtaaaagctatgatcagcaatccgatcattgtatagacaatgaatcggcttgatttggcctcttcttccactttaggctgctctactttcttttgaacaggtctattgggcggtgctatttcgactcggatgcctgctgaaggaggatttgacgcaggttttgtttgtgaggttttctcttgtttcgccttttcttctttaatctcttcaatccactgatttaggggacgacgaatacgtccaaaagaaggtgccttaataccatagtgaaggcgaagttcctcttcgatacgtagacaaagctgatgagccgcttcaattagggggagttcttccggcaggactcggtgtttcttcattcgtttttcagtgtactcaaccatccattcaatcgcactaatacgggcattagcttttgtgtactcaggataggcggagaaccaataaacaacacacaagtcgagcacagaatttaagccctcaactaggccttttagtccgtcattattaatggctgccacagtaaagtaacagctacagcgaaagtctttgcctttcgttctaaggatttcttttgatagtgtctgaactgtcttccatgacacacggcctgttactttgttcaagccatttatttgacgttttatttcatcaaagtcgtctaagccatttggattggtaccgcttggattactttcatcgatcggtcttcttgtgaagtcgctgaaaaacataatgttaagttccaactgagttgataataaaataataaagatagatttttttattgtattcgcctgctatgccaaatgacatgcaatgcattttttctgattttatagataaaaaaagatttttagacaataaaaagccgataaatctaattaaaatccattttgtgtgatctttgttcctaatattaatatcaattggctaatttgcgtgtaggaaaaatcctatttttcattcattttgttagttttttatgatgtaaatgaacattttaataaacttaatattaggtgtttttttattatcaattgctgatatacggtctttgcttattcaattttctagaataatgcggctatttatcaaggttctataatgcgcctgttaatggttgaaatgcctattttgatagttaaagtgtgctttgttgtggtcgctttattttatatcttgaaaaagagagtgtaattgatatcgtccttcctccaaataataaaaaatagattttattggggaaatcaatgtcaaaatctagtcaagttggactcatttatgccatgtgttttttttctacttttgcggtggcaaaaggggagcgatatggtgcttcagtcaatatgatcactgtagaaagtattgactatgacgaaggctctagtggtgacgtagatgctttacgatacggtcttgttcatactcgcccaatcgatgaaaataacaaccgttggcgctggtggcttgggttgaattatctcgcggaagatgtcaatgccccttcaaacggcctttatcaagaagttacaaattacgagtttcgtgttgttcctcaatacgctctgagctcgtggagcatatttactccttatgtcggtgctggcttatcgtttggttactcgcagtattcaaaccgttggaaagtagaccaagatggctaccgttacgaaagtgtcgaagatacagatcagtttgagttgggtgcagtcgttacatttggtacggccatcaaattaggcagtaaccctgatgcgcacattcaaattattccccaagcttcctatattcttccagtcgttaatgacggaattggtggagttgagctttctgtttctctactcttttaaggtttagcaatgcaaacacatcagctagtcctatttatttctaaatgcccagaggaatatacgggcgcaaagcatattgaaatgccagaaggtggtggctcagttggacgagcgcctagctgcactctgtctttaacggaccataaccgttttatctctggtacacactgtttgataagtgtatacggtgacactttctacattagcgatgtgtcgacaaatggcactatggttaatggtaataagattctaaagaatcaaccgatatctattgttgaaggtgacgttgtttcattagggcagtacgagattggcgttgcattagagcatatatccgctgttcaagatattgccgccgatattgccccagaaagagtatcgaatgatccactcgtcaatctcggtgaggcggtcgtagaagaggaagaaaaagtcggtgcattagaagacttgttcatggagaccaagcaagatggtgttaatactgacgatcctattgcgcatctaaagttttcgatgcagcgtgaagacgactatcttatccgcgatgttgaaaagcctgagcctgcaccggctaagacaatcgaaaatacgcgacaagtcgttgatgacagtttcagtatccattctgaattcgatattccaaatttaatccctgaagattggctgggaggcgttgccacgagtaaagaatctgttgaaccagcggcagagcaagctaagcaagccgcgttcattccagaagattttgcgcacacgaagcccgtttcgcctcaggctagtgtggttcaaccagacgttaaccaacctcaacagcctcaatatgaagcgccacttagtcacgcgacgtcaaatgttactcaaagagcagagcctgttagccagaaatgggaagaggtaacacaagcctttgttccatcagcacaacctactcagccagaaacgaaagcgaaagaggcccaacaaggttttgtaagcaacgagtctctagcgattgagagtgtggctcattcttcagatatcggtaaagcgttttatgaaggcttgggtattagtaaccctgatctgatcagtaacgaagctctgctgtttaaacaaatgggggcatgtcttcgtttgtgtatcgataacttgcaaaaagacttgcatgaagttgagtcacttaaaggtgaacagggtttaagcgaagcggattcaaacttagcggaactgatgttaacgctaaatagtcagaacttgctgtctccgaatgaattggtggagcaaatgttggatgagctcaacgatcatcaaattatttttaacaaggcactcaatgagttgctaattgaacaatcagagacgaatgaccctgtcatgtttgcaaacgacgttgcaagtaaatccatgttcaccaccaaatcaaagttatggggtgagtatcttgagttctacgccaataaccgtcgtcaaatgaatgagacgtcattgaaaggtttgatcaagaaaaactacaccaaagcgattaagggaagccatgcgtagtattgcaatattgctcggatgcgccttgctatccggttgtgccatgtgggatcaatttaaagaatcaacaggcatcacgcctgaaacgtcctcgattgagttggtgattgaggcgtcttcggttctaaatgttcgtgaaggcggacaatcctctccggttattttgcgagttcacgaactgacgtcaccagtgttgtttcgtagcttagacttcttcgcattgtttgagaacgataaagcatcactgggtgatgaatacatcaaacgttatgaataccaaatgcagcccggtgaaaagatccacgaatttttggaactcgacccagcaacccgtgcggttggattctcagtcgcgtttcgcgatattgacggttcttcttggcgtaaagtcgaagtgattgaagagaagagcgagtactacataaaactcaagttagaaggcagtgagttaatttctgacaacactcgcggcattgaacaagtttatttttaaggaataaaagcatgtctttatacaatccagttgtttggcaagatggaatgttcatgaagccgcagcactttcagcaactcgatcgttcacaaagtaaactgtcgagcatgctgagcgcaaatgcatcacctttgcattggggcattaagcgcttagagattaactcgcaactactggcgttgggcaaaattggtatcactcgcgctgaaggtattcttcaagacagaacaccatttgagctgccgctgctggctgaacttccagaagtgaaagacgttgatccttcgattgctgacaaagtggtttacctttgttgtccgttaccttctgagcgctctgaactattcggcacgaagaaagacggcgcacgttacacgctggaatctcaagaggcggttgacgcttgttatgattcagaagacatggcaaatatcgtagtcggtaagcttaacttctgcttgatgtatgaccatgaagataagagtgcttacacatcaattccaattctgaagatctcagaagtgaagccagacggcagtgttatcttggatgaaagctttatcccgacttgtatcgatattcatgcctctaccgtgctaaacaagttcgcaaccgagtttgcctctatgctgaaacaccgtgcagaatccatcgttcagcgcttaggtgtggttgaccagcaaggcgtatcttcagtttctgatttcatgctgctgcaagctctaaaccgatacgagccgctattctggcactttgcgagcgcagaaggggttcatcctgaatcgttttatcgcattcttcttcaagcggaaggtgagctttcgactttgtgttcagcatctcgtcgaccaatcgagttcaccaagtacaaccatggcgacctaaccagctgtctatcgcgtacattagatagtgcgaagatgacactaagtgtgatgtctgagcagcgtgcaattccactgacgttgaaagaccaaaactacggtattagaacggcagcgattccagacagtaagattatcgacaccacgacctttattcttgcggttaaagccgacgttactttggatgttcttcacacccagtttgttagtcaaacgaagatcggttctatcgataacattcgtgacttgattaacctgcaactgccgggcatcgagattaaaccaatgccagtcgtacctcgcgcattgccttaccacgcaggttacacctacttcgaactggataagactggcgaagagtgggcagcactgaaaaacacggctgcaattgcggttcacgtagccggcgactttgctaacttgtcgctgcaactatgggcagttcgtctatgagttacgcagaaactgacgttactgtagtcttattccaaccagagccaggcaaaccgatggaggtgatgccagcgcctgacttgtctcgtaatatcgcggtacaagacctaaacatcgaaaacatgggcatcaacccgttggtcgaccagttctcatggttgatcgccagtttgtcttgtatgtcttcgattccttggcttgatgacccaatgccgtttcgtgaacaagtagctcgtgagataagaaaaggcgagcgtaagctcaatgaaatggaactggatcgcgcatccattttggtcatccgctactgtttgtgtgctgcgattgatgagtcggtctgccgacaagagtggggcgctaacagccattggagccagaacagtttgctgtcagagttccacaatgaaacatctggcggtgataagttcttcgttattctcgagcgattaaaagcagacccgcgcaaataccgtcacgttatcgagtttctgtatttgttgcttcaacttggctttcagggcaaatacggccgtgaagagcgcggtaacgaaaagctagcagaaatcggcaatacaatttatcgcttagtacgtgatgaacgcttagcagagcaagagaaagtctctctcgttaaccttaaggccaaataccttaagaagccgttaaaaagggtgatttcacccaaactgattttaggcatcagtgcgattaccttcgcaatcatgtatgccgcgacttacatcgtcatagatttaaagtttcagcaattacttgaagtgtatcgataaacagaagatctcgaaatgagatcttctgccagttttaaggtttattgtgaatgtcactagcgatttagtgaaattcactagtcatttagagggattgcaatatgcgatctcttttattattcaatcaacaaaattcaaatggattggttatgggcgtaacagttggtgcaaatggactttcgatagttcataaaggctcaggtggtgaagcgaatgcaacgctcccagatgtttgtttaacgaaggtcggtaagccgatcgttcctatcccttatggcaacaatgccaaatcagcagatcttgctggtggcactacgactatctctatggatggtggcaacagtgtcgcaattaaaggcagtacgttttcaaaaagtaccggtgatgcgggcggcgataaaaaaggcgtcgcatccggcacgatagaagccgaagcaaaattcatttcagcctcaccaacggttaagtttgaaggcaagggcgtatgtcgcctgtctgatcaaatgaccatgaacaaagccaacactatgtgtttgggcggcgcacaaaacccatcggtttctgttacggaagcagatgatggaacatttaccatagacattgattatcgatacccagatggagacccagtatacaaagccgatttcaagattattgacagtactggtacagaacatgttggcactttaagcgacaaaggtaaggggacgattacgggcttaccagcaggttctatcagtttattcctgggtgaggataagagagatatagagccaatccaaaagccgcaaccaaacaaggattatcttgaactgccggggcctttggacttagtagagtttgctggacgtggcttggttgaatattgggatgctgttgaaatgccgagtgattatagtcaatgggcttggggagcgatcatgggggacttcaatcaagatcgatctgctggccaaattgcttttgatagtgctgttacagctataccattcatcgatcaaattggggacggtcgagacattagtgccaacctttatgctttttataatgcagacgacataagagaagataaagaaaagtacacagatctcgttatcactctagttggctttattcccaccgcaggtagcttgataaaagggttatttaaggaacttaaaatcttgggcaaagccgctgatttagatatgattgctgctttccttagagccggagcaaaaggtgatgtggtcaaatggatgcaaagccttgatatgtccgtcatcaaaaatgatatatatggtcaactagatgatattactaaacaagtaaaaacgttaatggatacacttgagcaagagagccgtaaaagagggtatactgttgttgctgacgcctataaaaggtgtgtggaacagctagatgacttttttaagaaagctgacggaccaatatctaaagtcttaagcgattttgatgataggctcaagatgatcctgcctgaggcacctttagttacatcagggacttcctttagtatttctgcagggacggctcaaggaggaggcaaagctgaggtaattaagactcgtaataagacgaaaaagaagactgatacgtgtccgttgtgtaataaaaagattggaaaagggaagaaagagtgcgaaggggcgaaagtcggtagtatcagcaaagcagttatggacgatggttcctctcaaaaattatggaaagaattcgaaatacgaaatggttggcgagacaagacagagcacccttggtggcacggggcgggagcagtgcaggctgcagctcttattcctaaaaatgcattcaaactaaaaaatgcaaagagtaagcaaactaaagaaaaaatgatgtttcttagacgaattgctcagacgtgcgcttacaacatcgacttttggaaaaatggtattggccttcctaataagaaagaaacagcgtgttttttgaggaaacctcgacatgcaggcggtcacgatcgtaatgaatttaactacacgaacgaatgtgtaaagcttgttcatgacaagttaagctcagatatcaagaaaagaacaaaagaaggtagttgttccaagacaacaaactcttcgttgattatgaagttcaatgaatcaagcaaaaatatttttgataatattgctgactttatctggtttattactcgtgatggaaaaaactatgatcctaaagtagaaccttacgtgggctgtagttcaaattgcaaaaaaatacacccgattattaatataaacactaagcgacaaattgaaaactatgacttagagattggaaaataatgatttcagatattaatgaagagtattatgtacttgtgtgtgggtctggtaaagtaccttctccttatgtttcttgtagtaaaagccactatttgattttccaagaacctatgtcacttaaaatgccaatccggatagagtcagcagctgaagcccaagagaaagttccattaatcgaagctatggctgccccgagtgatcctgtactttcaggtagaataacacaagtcctcaactactttgatctttataaagttcaattgctcccagctatatatacccataatgatgatagcgagcactcttattcagtcttagttgttgataacgatattgatgcttacgattttgataacggcctatatattgatcgtttggatgatggtgaggttggaaacccaagaaactgtcgacttgaccccgaaaaactttcaaaaatcccgttagaaaaacgttatatttttaaaatacggggcatgatggattatttagtccacaaaacaattgccgaatctctagtcgcattgaatgcaagtggacttcacttagttccagtattggaatgggacattggttttggaatgaaaatttaatggataaatcaacgatgcatgactatgaggtttttcaccgtagtaaatactaccaaggaaataatcgccttatgcaggcaaataaagagagaatgatggagattcaaccagatttagtggtagatttctgtcaatgtccacgtttattttttgcagagaaatacgctgatattctatctgacatggaacctttgggaatagagcgaattaagaacccgtaccgacctcacctagatgcagaagtgcagattgacataggtgatgaagaagaacacgagatcattgacgatgttgattatgtattcttccacgtatttaaccgctatgacgtattagataatgaggctactgtgtggaattgcgaaactccctctattggtgctgtcgtaactcctgtattggatgaaagtaagctccatgctataccccttcagcagcgattgatatttaaactcgatcaagacccctcatttatgttcgtgcatgatagtgttgttgatgccatgctcaatgccaatatgcaagatatctgggttcgttgggcagattgattttgccacttccaaattcagcgccgaaaggcgcttttttttgagcacatagcttcaatttatgtaatgtgttgagtattttgtaagaggagacgcaagctgaacgcttagccttctttttattatttgtttaagaagcatgatgtgatgccaattagtgaagaatattatgtgttatcaaatgactactcaaaggtgaattttgtagtaaacatggaagatgaacgtggatttagtttgttaatggataaagcgatatctacatccatgcatgttaactttgaatgtattagcttagagaattttagcgctatagatgctccatttgatgtttctgtcaacacggagatattatttgacctggattttcttgaagtgttatcagaattaacactatacaaaaattcacttgttccagctaagattaaagataaagagtttgtttatcttcattgttataatattattgatatagaggatgaagttaagggggttaactttgatctattatcagaaatacccatcgaggaaaggttggtatttaagccctcttatggggctatcgttgtttttttttcataaatcaattatagaaaaaaatcaactctgttaagagtttttcatattcaaagcatcaaaaggtttccgattggtctgttgaatgggaaatgtcatgaattatacagagcttaaagttgatgacctagctagcttggataattataaactccgacttgttaaagttgaaccggatatatatcccaaacatacgggctaagtgtctttataaatgattatgtcaaagggaaaatacgtcatgttgacgctctgaatttaactgagagtgttatatcaatccgttgttttaaaccagattgtgttttctataatataattattcgtatttacgattgttcttaacctggtttatgttataaaatttggagctttccacgtttaaaatgcaaatagcatacataaatgaggactttaacgagggtgaatcattatttactgccgttgatagtcggtcgtctttctatgatgataatgatttcactcccacttcttgtaaaagtgagattgtgtggaaagatgatgatatctatgaaattgaagcattagatttaatcagaggtggtgcttttaatgtcactaagcctattgctgaactaattatgagtttcgacccatacggcgttgaagtttatcccgcaaaattaacctgttccggagacggagtgttaactgagcgctacataatagcagtagataacttagtagatgttgttgattatgataaaggcataaaagtgactgacgtaactactcgtttttacttatctaatgaaaagatcaaggaacttccggatagcaagaggcacgtttttaaacctgtcggaatgacaaaaacgttcttctcaatcgagttgtttgaagcgcttgtccgtttagatgaggagggtaaaataaatactagtatcgttgccttttcatttgatacatccgaagagacgccccgagtctagtataagtaggtaaatataataatgactaactatatagttttatctaaacaatctttgactgacttcccattccagcaaagccctaagccaatagtgccagtagagccagatttgcttttggaaatgaccttttctccaaagctatttatcatcagtgatattgcgtcaaaagtagagaaactggtcgttcatggagtagagtggttagacgctcgcgtggattgttctccaagccagccatctgatgatgaaattaaagtttacgaagattatcgtatgccttacatccatcaaacttataaattgacggataaagaaaaacaatacggcaagttgaactggttagacatcgagtcaatagagtttgacttctctaaattagaaaatattcctttagaagaacgattaatatttaagctagaagaagactttggcttcattttcattcaccaaagtgtgattgaccttcttaaaaaagacgtcaaagacgtttgggtaagggatgtatagaaagtgccgaaaggcgctttttcttttaattacaatccaattactattcttaatagttgatgttatgtagataaaattcgcaatgggcttgtgggtaactgttcaattgtgaattcctaccaatttcacttttatctgaaatattgtcatcacagcagtcggctgaacaagtgaagtaccactt

SEQ ID NO: 2 - DNA, 56968 bp, Vibrio parahaemolyticus
  source 1..56968: mol_type = genomic DNA
agtgagcagataatgactaaaagatacgcctgcaataatagtgagtgcgatggtaataagaggcagagcaaggttatgattacgtgaacttaatattaaacttgtcattaaatgaaaactgtgcttagaagctgaaaattgaggggagtataatatttaaaataaaatcttacacgtcactaacgacaattcctttgtttttactaactaaataatgatagatatgaaatagcactaagaatagggttgaaaagtgtctgtttttttagtttttttgctttattttatggttattttgtcgcgttgagaaaattccacacaataatataatctcggcaaaaatatactctgccagattattatgttcactaaaaatacgctaaaatcaacgttaggtctcatggctacttcactattattgagtgcatgtggcggaggggacggtggcgataatacgccacctgcgaaggaatacaggaaccatctcaggtaatgtttttgacgctccagtcaatggtgcaaaaattgaggtgtttgagtttaaagatggaaagctaggaaggaagctagcgagcacatcttccaatgcatttggtgattacaaagtcgaatttgaatcgtcttcgatgccattgtatgtcgtcgcacaacaaggtagttacaccgatcctttaacaaaagagattgtgtcctcgagtgttggtaaaacgttgcgtttagaaggcgtggtgaacttcagtgaaggtagtaaccaaaagttgatgttgacgcctctgaccaatattgtttccggtttcgcaaaatataaaattgcaggcggcgtgtctggtccggacgctgtctctcaagctcttgatagtattaatggcatgtacggttttgatgtcaatgaaaccacaccaattgatattactaaaggtgggcagagtagctttgcaacgccggggcatcagtatggtgcattgcttactgcatactcttcttactcttatgacatgatccaaaggtacggtcaatccgaagacaatgtatatacatcaatgcatttagctgatgtacagtttcgtgatgtcattgcagacggccttcttgatggcctagaaataagcgaagcaacgggagctgtcacaccacttacatttggtcagcagaaaatcaccagtgatgtgtacactcaagaactctctcagcaagttttgatagttgtaaatgaccctacactcaacatttcaggaacaaacgctgacgattacgttgcctttagccaacaaataaatagccttggaaccgctggcgaaacagatggtgtcattccacctcgtgatgacacagatattgataccattccgccaactgcaactcgaacagacaatgatgtattggcaggtaaagacatcgtagatatcaaaatcaatgacgatattggtgttgagtcagtgtctgcatttgttgaatacaaactcaacggtgcatggcaaggtgagtttcaatgtaatgatcagctgacgtcaggcagtaagttttgttcggtggatgcgcaagggtttgaggttggtcttcgtgaaacgaacatcaaagtaatcattgatacaaaagcgatcgatgctgtttatgtcgaccaagaaactggcctttccaaggttactgctgcacgtttagtcttatacactgcagatgtgctcggaaataaattgcttcctggcggtgtaaaggtcattatgtcaatttctcttgggataacgacgcgccagttatcaccgtgacctcgtctagcgctatcaataatgagcttcaagaatatgtacttaaaggtcttgtaaaagaagcgtctcaggacattaagtccgtcagtgtgtcatttaagggtggtttgcctgacgatgttgcttgttctcccgttacgtcagagtcgggaagtgcctgtgaatttagcaaagcgtatttaacagaccagttcttatcaactaccacgtttgatataaaagccactgatacaaaagacaatgtcggtgagtttcaacatgcggtgagccgagatgatcaagcacctgcacagatcatcacatacccggaaggtacgcccatgagttacgtcaacgtgagccttaatggagaacgaaccacttacgagggcgtatattcacaagatacttatacagctgataacgtgcagtccagccgagattacttgaaaatagattatgtctatgcaagcgcaggtcttaaatccattagtgggattgatttcgcaaacttcaatgttaacttattgaaagaaaaaaagattccttacgttcgtgtcaaaatatctgatgcgagttcagagactgtattaggttcaagtgctgacaaacttaagctcgtcgtgaagtattacgtaagtcaaaacaatgacaacaactatgcgttccaaaatacaacgcagacgttagcttcgacggacgccataaccgcaagcattcctcacgaaaccattttggatagtagcggacgagttgatgaagttatttactatgtcccttttgtaaaggagatcctaggtgatggcttcaaaaacgtctcagaaaacgcgagccaaaaactggtaattcaagctattgatgagtcagagaatgaaagcacgactcaagaagtttatttcagaagcagtttcgacttgccgacgatgactatcgtgacacctttcattggtgcacgtgttcaattggaaggtttaaaccctaacggagagtttacgtcattagctagttgctccacgattcagcaaccggaaagtgatgagtctgccgcgtttgatgttgcttcatgtgagactacaaccgatgttgttaattatgattttatgcgagttcgcttaattggtgttgatggaactgatccgcattactatcaatggaaggataataaaagcgcaagagctagcgttaacttaaacgatgcaaatataggcgcatacttcgaactgaacggttcgaatacttattatgttacggagttatctacttaccaaacaggcctatttgactatcgctggaatcaagttgaggccggagacaaaacctcagagcgcgcggctctcattcttacggatgtgaagtatgcattgtccggtggaaacgattcatttttcggttttgatccaacagtgacatcttatgcgactaacgagatgttagaaaacccaattccagatgaactgtccaacgactacttgcatcgattcctcgttgaagcgatcgatgatttagctcaagacacgccacgtaataactcttctgattttgcttctgctatctatgatgatctgagctatgacggtaaggcaaacggtattggatctggaggtaatctaattaaattagatagttatgagtttagttctgacacttaccgtaaagatctcgctcagtcttactacaacatcatgacggaaaaatacggaattccaaccaatattgcccagctttatgctgacgatatatcgcaggcaaatccaaagctagacgaagacgctatttttgatagtgagggagaaagtatagataaagaagcgcctaaacaaacgttaacaatagagactggccgagaaactatggtcggcaacaaacgttacgtagcaggcgaagttgtttctaaaattgtcttggaagatccatcaggtatcgttgaaaatacggaaaatgcgcctacattcaagagcatgtggtatgcacagagcaagcctaataccgcgataccattggaactgagaattgatgagtctggcaacagcaataaattccgcaaagagtatttgtttacactcaatacgcaatcaacagacctaatagacatcatcaagtttgctctggagacaacggcggtcgatacccaaggtaacgcttataccgatctaaacccatatattgaaactctatatgttgataatgactatcccgcagcgacttacattccaccaaacgacttggaagggaacccaattggcgaggatatttatttaaatgcgaattcagttcatgagttgacatttaaacttcaagatcttgtgggggaccaactggataagcggagtttatcgtttgtcgatctctcaaatggtacaacggtcgattacacaagcgatcagttttccagtaatacgcaagattcatttaaggttaaattatgtacaggtactctttgtgcgagccaaggtaaagtcgtttatccgggagatggtaactggactgtcaatgtaaatgcagaagacaatttggggaactttgttaatcgtcaaactacaaattcaccaaaattcaatatcttaattgactctgaagggccgattgttaatggaggaacaatcaataagcgtttgggtggaaactcatcttgggtgccggatatttcttggggtaacttgagtccaggcagtgtagttgaaattgatatgaagacggagtttggtaagcgtattactttgcaacattatgagactgaacctacagacagtgtggagccttatctattcggagagcaaccaaacattgaggttcgtttaatagccggggcgtttaagtatgaagagacgaactcgttttacgtaaaagcgaaagacagtgcttaccctcaagcagagtcgacaagcgaatttgtttttcaagtagacaacaaaggcccggtaattgaactgtctgaaccttggatcgtagacaaaaactctgctggtaactacgtcctgggacctgattttcaagtgaagctgagttcagtcattgatgattctaaagtaagtaacgtcgagctgtggcaacaagctggtgactctgcaatacagtcaattgtacctagagatccaacacaaccatttgaaatggaaattggtcgtatacaaagtgacagtatcaccattgatgcaaataagagggcgaacctgttcattaaggcgatagatgagtatggattcgaatctcagacagagccacgaacgataattattgaccgagatggtcctacgttaactctgaatgggtttaactcggaaagttattatcgtggtaactacgtgttttcactttcagctcaagatctgagcgaaacgggaacgccttcatcaaatggtgtgaacaagacgacattggaatattggagctttatcgatacaccttctgataccggcacacaagtagatgaagatatgaaagtgagcttagaaggacagaatgatggtgatagttcaacccgtacaatcaaactcaggggcagcgatgttcgcgggaatacgacagaggtagattacgcgattaaagtgcaaaacaagttgccaacaattcatagtgtgtctttttcctatgaagatggaacgccgattactggtgggttagttacgcgcaacgatcctgttgttattacccttgatgcggaagatgcaagtggaattcctcaaatcgacggcacttatagatacggcgatcaaaccaataatatacagtttagtgaagatgaaacgggtaagtggaagggaagactaacttctgcagaattatctcaggatggaagctatgaactaaagttcaaagtttataacaatgttttgtataactcaggcgaaacacggccagtagtagaacgttcggaaagtattaatgttcaacgcgaaggggtcgtgttatctgtcgcgcaacctgaaaattttcaggctcatatctcgggtaagacgttgagtgtcgagttcaaccctgtgggtgatgttaaggctcggactctagaatgttgggtaagagaaaattacacctcaagcgaagctccaaatgatggattgagcaatgcttactctggcgttatcactaatccacaggaaagttataagtgtagtgtgacgacagatgtaaacatgtctactgcacctgtcgttctaattactaggacagtgggccagaacgatacagagacggtgagcaaattcagctttaatatgatggatattgaagcaccagcagcattagaggatacctatctatttacaggtaacgaagttgaatttgatgccgatggaaacaaaatgttgagcttaacattgagcttcaaagataacttatcaggggtgaatactgggccaacgaaacgaccgttgttagttagaaaccgttttaatataacgtttgaaccaaaaagttgtagcgtgagtgctggggttactacgtgtacttacaccgaacgttattcggatatcatcgcaacgggggtatcacaacatctatataccattaaaaatttatctgatatggcaggtaatacatcctcagatcattctctagaactattgttgccacctccaggttcagttgatattggaatcacaagcccaacggatgacagtatcattcgaggcgctcagctggtgatgaactttaaagtgaaggttggggcacaatcaattttagaaaatgtgattgcaacagtggatggggattcatataacctcaatgatcatccagggaacttttcacgatttaacgattgtggagagggctattcctgttcgacatttgaagcgcctctaagtgcgactttggatggacaagtgatcaaaccgagcatccaagctattgatgtgtggggagactcaagtagcgataacgtcagtgtgatggtcgataacagcaaaccaataattgatagtgacgctgttgtcagtgaatcccctacagattcaagcttggttaggttccagttttctattagggatgaaggaagcggtattgcaagcgtcaaatacacaaccgtcaaccctagccatgtttttaataaagaggaaaatgcagataactcacatctttatttcgagctgccaaaaagtgagctgcaagggctgaatcaaatccgagtaagcattgaagcaacggataaagtgggctggagtgaaagtaagactgtggatattgacgtgtctgtacctactattacggtctcatttaatggaatgactagcttgcaaggaggcaaactagcttttacaaaagaaagtcagttattcaatgtgacttcaaaagaaggacatgatgtaaaggcatcaaattactctatcgatttagttccaagtacaggtgatacgctaaatttttcagggaatatcgtctcatcgacagggtctgatactatggtttttgacgctgaagatcagaccgaatatatgtataaagtgacggtcaccgactcaatagggcgttctattacagactttgaactttttagtacaacatatggtgccaaaggcatcacatctatcgttgactacgagttaccagcaatatctggtgtttctgctgatcaagtatctatgacaccaacggacggtaaatatgatttagatgtaacagcgtatgttacagataaaaacttgagttcagtgacatctacagcagataacgggagtggggtaaaagtgggaccacaatcagttacagagcctgttagtgtagatgatccttatgtgatgcactactcattgactcctggtacctatacgctacaagtagccgcgttggatttggcgaaccataagactgagaaatcaacaagcgctaccgtaaaggcagcaaccgttccagagttaacaatatcaactgcatcacctagcccttcggctggtggagttgaaatcccgcttacatttactttttcagaagaggttaaagaatttagtttctccgatgtgaaattagaagcgagtgataaaaacgaagctggagagcttcagcaagtatcatggaacaccagtgacaatataacttggaccgttacctacacaactccagaaaagcaaaataaagaaataatcatacgggtcgacgatgatagttatgagagcgtcaactctatccccggaaaaggggatagtttgattttggaggtagaaggagtattaccaacgctgtcaaaagtgacttttgatcctaagcatcaagcgataggacagtcggtgaacatcacacttgagttcgataaggaagtgcaagaagcgacagcaaaactgggaaataatgatgttacttcactcgttgctacagcgaataagaaaatttggacggggagggctgaagtccccaatattccagaactcaatgtcggcctatttgtaagtaagtacaaagacttgattggtaatatgggcgaagatgattcttccaacgcaataccaatcacaccaaccttggcgatcacgcctgtaggcaatgtggatagcagcaatgccgccgcattgcaaatcaccggtacttcaagccgctttgatggccaaaccgtgagcgtagaaatcaaagcgcaaggcagtgaaacggttatcgccagcggcagtgccacggtacaaagtggtggcgcttggacgagcaacgcgatggacatcagtggtgagccaaatggcacctacaccgtcgttgtcacaggcaccaatgcctcaaatgtggaagcaacagaaagcacaaacttcacgttggcacaggcgttgccaacactgactaacgcaacattcaatccgacgcatcaagcggaaggacaaagtgtgacggtcaaactggagttcgataaagcgctgcaagcagcaagcgcagagctgggtggttccgccgtcacactgactaagacagcggatgcgaaagtctggaccggtgacgttgtcgtaccagtgtcgtcagagctaaccgtaggcttggtggtgaaagattaccaagacctttcggggaatacgggcgcagaagaccgcaaccactcgatgccgatcacaccaaccttggcgatcacgccagtaggcaatgtggatagcagcaatgccgccgcattgcagatcaccggcacttcaagccgctttgatggccaaaccgtgagcgtagaaatcaaagcgcaaggcagtgaaacggttatcgttagcggcagtgccacggtacaaagtggtggcgcttggacgagcaacgcgatggacatcagtggtgagccaaatggcacctacaccgtggtggtgacggggaccaacgcatcgaatgtcgaggccacagaaacgagcaccttcacgctatcgcaggcattgccaaccttgagtaacgcgacctttaacccaacgcaccaagcggaaggacaaagtgtgacggtcacactggagtttgataaagcgctgcaagcagcaagcgcagagctgggtggttccgccgtcacactgactaagacagcggatgcgaaagtctggaccggtgatgttgtcgtgccagtgtcgtcagagctaaccgtaggcttggtggtgaaagactaccaagacctttcggggaatacgggcgcagaagaccgcagccactcgatgccgatcacaccaaccttggcgatcacgccagtaggcaatgtggatagcagcaatgccgccgcattgcagatcaccggcacttcaagccgctttgatggccaaaccgtgagcgtagaaatcaaagcgcaaggcagtgaaacggttatcgctagcggcagtgccacggtacaaagtggtcgcgcttggacgagcaacgcgatggacatcagtggtgagccaaatggcacctacaccgtggtggtgacggggaccaatgcctcaaacgtggaagcaacagaaagcacaaacttcacgttggcacaggcgttgcctacactgaccaacgcaacattcaatccgacgcatcaagcggaaggacaaagtgtcgcggtcagactggagttcgataaagcgctgcaagccgcaagcgcagagcttggtggttccgccgtcacactgaccaagacagcggatgcgaaagtctggaccggtgatgttgtcgtgccagtgtcgtcagagctaaccgtaggcttggtggtgaaagactaccaagatctttcggggaatacgggcgcagaagaccgcagccactcgatgccgatcacaccaaccttggcgatcatgccagtaggcactgtggatagcagtaacgccgccgcattgcagatcaccggtacttcaagccgctttgatggccaaaccgtgagtttagaaatcaaagcgcaaggcagtgaaacggttatcgttagcggcagtgccacggtacaaagtggtggcgcttggacgagcaacgcgatggacatcagtggtgagccaaatggcacctacaccgtggtggtgacggggaccaatgcctcaaacgtggaagcaacagaaagcacaaacttcacgttggcacaggcgttgcctacactgaccaacgcaacattcaatccgacgcatcaagcggaaggacaaagtgtgacggtcacactggagttcgataaagcgctgcaagccgcaagcgcagagctgggtggttccgccgtcacactgaccaagacagcggatgcgaaagtctggaccggtgatgttgtcgtgccagtgtcgtcagagctaaccgtgggcttggtggtgaaagactaccaagatctttcggggaatacgggcgcagaagaccgcagccactcgatgccgatcacaccaaccttggcgatcacgccagtaggcaatgcggatagcagtaacgccgccgcattgcaaatcactggcacttcaagccgctttgatggccaaaccgtgagcgtagaaatcaaagcgcaaggcagtgaaacggttatcgctagcggcagtgccacggtacaaagtggtggcgcttggacgagcaacgcgatggacatcagtggtgagccaaatggcacctacaccgtggtggtgacggggaccaacgcatcgaatgtcgaggccacagaaccgagcaccttcacgctattgcaggcattgccaaccttgagtaacgcgacctttaacccaacgcaccaagcggaaggacaaagtgtgacggtcacactggagtttgataaagcgctgcaagcagcaagcgcagagctgggtggttccgccgtcacactgaccaagacagcggatgcgaaagtctggaccggtgatgttgtcgtgccagtgtcgtcagagctaaccgtaggcttggtggtgaaagattaccaagacctttcggggaatacgggcgcagaagaccgcagccactcgatgccgatcacaccaaccttggcgatcacgccagtaggcaatgtggatagcagtaacgccgccgcattgcagatcaccggtacttcaagccgctttgatggccaaaccgtgagcgtagaaatcaaagcgcaaggcagtgaaacggttatcgccagcggcagtgccacggtacaaagtggtggcgcttggacgagcaacgcgatggacatcagtggtgagccaaatggcacctacaccgtggtggtggcggggaccaacgcatcgaatgtcgaggccacagaaacgagcaccttcatgctatcgcaagcattgccaaccttgagcgacgcgacctttaacccaacgcaccaagcggaaggacagagcgtgaccgtcagactggagttcgacaaagaactccaagcggtaagcgcggcgttgggcggttcggcgctggcattaacccaaacggctgacgcgagtgtctggaccggtgatgctgttgtgccagtaacgtcagagctgaccgttggattggtggtgaaagactaccaagacttgtcaggcaacacaggcgcagaagatagcacgtattcgatgccaattaccccaacgatcacgatcggaaccatcagtgatgtatcgggtaatacaacagtaactatcaatggcacaactactcgttttgaggcgggggagataatagcactcaaagctgttgataccgatagcttggtggttctaggcagtgcaacagtgttagttgatggtacatggaccgttgatcttgatttgtctactttgaaagacggagtaataaccgtttatgcaaatggtgccaattcactctttgcgaccgctgatgaggtgaatacaacattcaattactctagtactacagcgcttgttgttccaagctattgggaaagatattccgcggagttaccttcccagaaagcagcctaaaggaaagcgagcattaagctcgctttctttttgcttgtatttaagtgatctagttcaaatgattatgcttttttcataaatgaatcagataactttatcgccttaatacaaggtgaaacatctagaatgcctagaccatttttgtttttacgaggcaatactgtgattgtgatggtaattgatgatcttcaatggtgaatatgccgtgttgataatatctattaatattatatagttacatgcacgtctataccctaggtgtagagttctgcattaggtagtgattactacctgagttgtgtatctatgatgaagtgaaattagcctagcatcgcttcatattagttacacctttgtgggtgtgacacgcactcacctttttattcatacttcctcagtcgcgataggttatttattgctactgaggaaggcgtatccctccagaacctcaaccttcggctatcagtgcttaaattgaatttgttaacttctgcccaaaagtaaaaaactgtttttgcttaaagcctgccttttgagttggattgaagggtaataagtttgaattgatagttaaaacaaccaaataacctgtggttttgttatttctcgggcatttcggttttctggaagttttctatgtgattgaatgttatgaattaaattagtagcattagcacaccaaatcgtacttgtaagccttatcagcttaaacgtcaccgcaaccatgtttacgatgttggttgtggagaattcaaatgcatttagtttgcaaatttatacctagctcgaagctttcctccaacgagctatcctacgtccttacacctgatgagtgtatagggcagctttcgcgtgtgcgaaattcagaagacattctcagaaaccttcctaaagagcttgctcagaagatctcaatctctgccaagaagtctagctccgggttgctaacggctattcgacacgagctaggaaatgggaactgggttgcgttgtcttctttctctcgtagaacgccgttaacagatacccaattacaaagtttcccaagactaaaagcacagcttgaatcggtctcatcgactggtgagagcaaagtttataaagcgggttataagcaggtaaaggacgacgtgactttggttcgtagctacacgcatgtcccttctgagccaagtccagaccaaaaaatcgttgtcgagtttgcagggcagtggtctagcaacgcggcatgtttgatgttgggaaaaaccgaagctcaaaaagaaaaagttactgtaggcaaggctgatacagaaaacaaacaccgcagcctcgcgacatttaaagatctcgaagctgagggcaaaacgctctatatcaaaatcccttgttccgaccagcctcagccaatcttgcttaaactcgccgaagacctgcaacccgttgataaagaaactcaaatggatgagtgggataacgtgttggtgcctgtggttccgctgcacttccctggtagcgataaatcagatgaagctgcagaagtgtttaagtcaggctacgtgtatgttgtttggaacaataaaatttggcgcgaagtggcaattacggaaaatggttacttttcagatacggacattaatagtgttcgtgaaggctctagacccaaaaggcacgccgatatctatatgaccaatcctgaaacaggcggtgttttcgcttatgagcctttccaaatcgtgcagaacggcaaggtggtaagtgaaggctctttgaatggctctggagaggctcgagtgttcaacttagttgaggaagaagttgagatagtcatgaccggctatgagccacaaatcaaagaaaagattgaaacaaatttgagccctattaatgcttctagcccagtggggcggagcgcgcaaggttaccctctcccgcatatatggcttccttacaagattaaaggtgagcctcaagaagtatatttggcttataacagtaaacgattgagcgagagtgaactatctgaactagaaagtgatcccggaaccaaggccataaaagtcactgatcttaaccattacagcagtgagaaaagctttaaaatgggagacggttctgttcgtctgctctcagtattaccaagtgctgcgacctcaaaacctgaaaaatacgcaatgttacgaagccagataaataaaaatgtagcggtcgtgtatttacttaaatctgtcgagatagttttcgaatatccgggctatacgacgctggacgagtcagatgattactttgagttaaggcaaagcgatggtgattggtcgcagcgagtgtgcttgcgccaatgtataaaaaaagagaatggttctaggttgattcgttttactggttggcctgcagaggttaaagaagtggatcttctccgtggttatcagggcaatagtcatcacggaagagataataaaacggtcatttttgctcaaacacccattgcggatttactggcttataagaaaaaagatcagccaagttaacaaaacaacttattcgagttgcgttgtcacactattaaacgcgatcgagaaagataacgaagagaatataagatgaatctacgtcatacattatgcttgattactttagcggtcttatctggctgtaatcaagaatcggccaaggaaccagaacaacacgcgccaaaacctcagacggttcaagaccgaatcgcagaaaaaaatgctcaacagtgggaagatggtcgtgtttatatcacagaaaacaattacgttatttatgacaaagagacgggtaaacctaaacactttatttttaaagctaaagaatggccacgtgaagatctcaccaatgaaggctcttatgatttaccacgtgtagtgtttcgttgggataacgcccgaaatgaagcgaaagaccactatgatcaaagggataaagtgtggagtatcaaaacggatgggacggacttacgtctggtcgcagatgagtttgttggcaaagttcgtctaatgcgtgtctctccaaataatcgctacttggctttggcctattcggcctcagaagggatgtttaaagtaattaaagatcttaagacgggtgagtatattgaactaggtcgttcacgtggctacccagagtttctatgggctgaagacagcagctatctttactacgtagacaagtttaaagactggaagtacacgttagcgacgggtgaaaaagaagaaacggaagttaattttaatgaatactctgttatatataatggaaaacgaattgttgttgttgcatatggagtagcagttttcgatgagcagacgaatgaactgctttattctgttgcacctaaaaaaagaggaggtgatttggatgccaaggagttccgtaagaaagcaattagcccaacaggacgttatgtttggtcagaaaccagaacgcaccgttatcttattgacgttaaaaatagaaccttccagtcggaagaaattgttccaagagcggtaggtaagttacgttattttgaaattctaggtaaagatgtaaattacacacgagatggagcggctagagttatattgagcaagacaacagaaaacaatgaatttggaccatacctaaaatgggagcaaattgggacaggccatagtgctggtaattcttctctatacaacgcttttgccaataatggtgattttgttaaagagggggatttatgagccttgtagagaactgcaatgcaataccgctacatgctagttattatttatttggcgcttactttgcagccgaagaagatgaaaataacgacaaaacttacccttcatggcactccaaaacgagtctagttggagaaaatatttccaatattcgcccgttagcgaaagtgccaatgcttaacgtgactgataatgaaaaccctaaactgtgggcaactaatcatgagtttaagttatgttcactgggtaacggaaagcccgatcaatatggggcttttgaggtcctgacaccagagcacacaattcatgctttaggtagggatctacccaaagagggtgagcagaccttcactttccgctttccgcccttgcctttaatagcttcactttctgagttaaaccccaaagagttagctacaaaagttgcagagttgagtagccagttggaaaacagcgaagcaaaactgatcttgacagagatcagcattgaacaattggctaagccttacgaaccagctgcgaacgggggactcagcgaagaacaggcagaatataacaagtggtatgaagatgtaagtgaatcgggagagacactaaaaggcttcgttttacctgttgtggcgagttgcgctgtagttttttctgatcaaggttatgaaagtgcgacggttactttgcaaaaatttgattcaaccaaagaaggtggggaatacaaaacaataggttcttcagtacaagcagcattacctgaagatgagcattcagaagaaaatgctaagcaatacccatgtgcgatgtttcacgttgatcctgctgattttaaagaaggcttatacagtatccgtgtcgaatttattccaagccagatgaaaaatggcgttccacagtttattaaagacgctcatattcagcaattttctgaaggtttagatacagtctacacttacgagctacgtgaacagattaccttttcttcaagcactccgttagggaaatttgctattgtcagtggcgatatgatctccctcgaggaatccttgatttatcaataccctcagtactttggaaatatgaaacattaccttcacggtaatgtacaaggtgcaactgccaatacgccagcttcaagaagtattgcgttactcaagaatatccgagatgtgtctgctcagctaggagctgggctgctgagcggcagcttgcaagacactctagaccgtgatggtcgccaagcagtgtttaataacgtctcgaaactgtattgggaagcagttaagtccgagatgccgcctgcgatgaaagcggcgggcgaattgtattacggtatttacagtacgaaagaagcgcttgataagttgcaggaactccacactccagctatcgcgggtattggttggaaggcgctatttcacgataaggtttttaacaccaatgaagctcgggcaagcgccctagctctttctttagaatcaagtctgggtaaccgacgtgtggttgccgggcgagttgcggaagcttggctttctggtggtaaaaccgttggttttaatggcttcggaacgggattgacagcacttagtttgtataacaaagctaaacagctggttaatcaaaataaaaaggttgataaagcagagcaaggaacagaagaagttacaaaagactaccttgctcaaattccagtttggaagacagctaaccatattcaggaagataaactcgaacaagctttaaaagatgcgcgtgatcaatctggcaatcagattgacgccattctcgtaaatagctctagaggcgcaggaataaaaatagaatttgcctttaattcgcgtgaaaaagagttaaaggatcatgcatctatatttaaacagcttgctgcttctcttgccaacgttttagaggatgagccaaaccttagagtagagattgaagggcatgcttgccaggtcggtaccgagaaagcaaacatgcaggtctcggcagagcgcgcagaatatgcgaaaaaactgctcatgaaggagtttcctgtttttgacggtcgtgtttcgatagccgcttttggtgaaagtaaaccggtctatgttcccaagcaaggtgaaaaagtcgaccgcaataaccctaatctaagacaaaaccgtcgagttgtgattcgtatttacttacagtctctgaacgtattctttcatcctagtcgctatggttcgcatacaatggaacgctctaggcttgctctagaagctgagatggttgcgcaagataaagcagaagtcgccctgcgaatggcaatttttgacagtgtcgttgacgtcgcttcctatattcctgctatagctcctgcggcaagagggtttatgcttgccatagagggttcaaagacagcaatatctgcggtgaaagtgattgatagtctaatgttggactccttcctagaagagttaaagcaaagccatgaggttgtcagtgagcttgaaaggctggcaaaaatacatatcgagttacttaacgatttgcgacgcagtaaggtagatgtaggtattgaatgtaggtcttatcaggaactcattactcaccttgagagcgaagaagttcgtaaggaggtgttaaaacgctaccaactacgagcgcttgctattaatggcttagtgttattactcgccgacctaggagctcaaaccaaaaggatttctaacatctcgtttaggttcttggttgagcgttacaaggtgaaggaatatattgagaattacattttgagcgatgattggtctgtacatacgatcaaaggaaacaccttagcggctaactggaagaaccgttgtgataacgagtactacaccgaattgtcgacaaagatgccacaaggtgcgcagcattggcctgctccaataacaactaaaagcaacgtttctcatctctctataaccgaaaacagggatagctttaaagcgtcaggtgcatttaatcaaatcttcccgatacaaactatgttgtttgaacacccaaatgaaacgatgtttgataactttaccgagtcatttaacccgcagggaagtgagttaaagaaagatgaaatcggctattgtcgaatacttgttcagtcagctcgttatactgaaagtgatgaaccagaatggatgtcatatgacagttggattcagcaatctagcaacagtcgcctaggtccatacgataaagtgaaagtgcagctggtacttaaaaaagaacacaagtatgcgaagccagttactattggctacgatcgcgtcgacgggctcgaaaatattaaaggtccagctttcagtgactggatgctgccaatgaaggttagtgacttcaacgctgatcctgatgggcgaataagtgctttttacaagcaacagggggttgatgacggcgctacgttgattgctattgagcatattccttcattccgcttcggtgatgtgatgatcgacggtatgaaaccaatgacctctaaagcaaagctaatgttctctgatgcaatgttcagtatggcagtttccggagcagcgatatcgggtcagccaagtgactattttgcagaagcggattcctttaaacgctatgtaaaaggcggaggcttccaaaatatgcgttactgcttgggaattagaacgaatcaagggcaacatagtttttatttaccacaagagtacagtctggggcagaagtttgatgatgctgaagatgagttgcaaattggcgctttaagtacggaaactagactggttttacttgatacctatttgggagagcagcaactgcgtctacgtgaacaggaccttatggtagagagctttaccttaatggcaagtgagggagacaaaaataccatccctatacttcatggcattaaacagcaagtggtggctatagacaccaaagcgtctggtttgaactttttcagtgaaagaaaatggtaccaatcggctgataatatctggcgtgatggtttctcatggggaaacaaaaagaaagacgatcctgccagtatttatgtgctgatttcgggtgaaagtcatgaaaaagcactttatgaacgtttgggtatgccgtgggaagcagtgaatatgtgtattcaactggcgctcgacggaaatgatggcaagccagttaaaggtcctctttacttcaccgatatgtatcatattggtgagtttgcccataaaaacgaagaatggatgtttactgaatctatttcagaaaaccatcgagaacaagttgatgccatgagtgagtttataggtgaagcagtaagcagtctcactaaagagaatgatctatctaagagcaaaaactatacgatgtactgcatgaggtttaacttggaatatgtcgcaccaacgggagttaggttaaaaggcctaagaccgtttggtggtgtaattggtggtaaagcgaacttggagctttcggttgctcatctaaaacaagtcggattaaatggagttgataactatgatattggtcgtgatgctgtaatttctattccttctttaaatgcggatagtgctttggcaggtaatttctttagtgatatgccttggctaattgagaaagagtcgacagaggaaggcgtagacaagtctgcggctgaaacttggaagcagtataacgaatctcaacgcaagaaatggctgaaagattggattgagaaacaacctgcggtaactcaagcacccaagcccttggaaaagtcacttgataacccttcctagatgcaaagaaaaaccgccttcgggcggtttttcttttcttatgacttcatactgaatttagaattttctttttaagccccatcaacctgccgcagaaatgtcgtatttcttcatcttatggttgagagtgctcattggcagcgccaattgctcagcggccttcttggtgtgccaattacacgcatttaagcaatcgatgatcactgtacgctcgtattggctgacaacatccttaaggctctttgaggcgtcttctaaatccgcttgtgcctgccgctcgtaagcaggttggtctggggtaggtgaggctgctttcatgtcgatatttggcacatcttctgggatttgttcgccaaactcaatgtgagtaatggtttcaaaatcggacagtagcaccgagcgctcaatgatgttcttaagctcacgcacgttccctgggtattggtaggcaaggagctgtttacgtacgttggcacttaaccctggcgcttgcggcaaacctagcgtgttggttgtgtgttgcacgaaatgttcggcaaggggaataatgtctgacttgcgagctctcaatggtggtaatgtaatagggaacacattcaaacgatagaacaaatcggcacggaatccgccatctttgatttgctgcataagattacagtgggttgcagcgataacgcgaacgtcgacttcaatctctttgctggtaccgatagggcgaactttacgctcttgcagtacacgcagtaacttcgcttgtagtagcattggcatatcgccaatttcatctaggaaaagcgtgccgccgttggctgcttcaaacaaaccgactttgtctttgtccgcgcccgtgaatgaacctttcttatgcccaaacagttcagactctaacaactgttcgggaattgccgcacagttttgtacgatcaaaggctgttctgaacggttggagttttcgtgaatgtatttcgcgatcacctctttaccagcgcccgtttcaccacgcaacagaacgtctacaggaagagatagcaccttatcaagacgattcaagacattgagcatttcttcgctctctgcaataggaccttgataggctttctgagtacgttttttgagctgagtgttctcgcgcactagggcttgattatcggcactgaggcttttaatcacttgcccatattgctctaaccaaacggcttggcgaatattactcgcagccatacgacaaaactcagtcaaggctgcctcgttatcgatgacgttcaaatcgagtaagactaacagcccaatggttttgctttcgctatcaatcaaaggccaagccagtaagttctcgctttttagtcccaatgtctgctctgtttgataaatgctttcgcagtcgtagcctttgtatttgtataactcgttaatcaaaaccacttcaccattacgaatggcaaagttaaatgggtcgttctcactagcggaatcaatctgcaatgcctcccaagggtgggaaacaatcgttttatcgttatggtgagcggtacttggaatcaacgcctgtcctgtctggtccaaaacataaatgatgccgtgctttgctaaggtcatttgtctcgcagcggtcaacacggcatccaatgtttgggtaagctggctgcgatcagccaaagattggctgatcgcaagtagggcgttgctaagttcgctatggttagctgaacgcataggtcagtgttccttgctcatcgacagaaactgtgatttgcgtgtgtgaatcatccttatcatgcaccaatagctgtgtcgatagctgaggtagtaactgacggttgatgaccgcgtcaatgttacgtgcgcctgtttcagcaaggcgacagttacctagtacgaactcgaccaagctttcttcgtagctcagggacaacttgtgatggctatgtaggcgctgagaaactttgtttagtttgtggtggataatttctgtcattgcttcgtcagacagaggcacaaacggaagtacggacatacgtgccaacagcgccggcttgaagtgttggttcaaagtagggcggatggcttctgcgatgatgttagcatcaatttctttagactggtgaaccagagattcaatttcgtgcgtcgcaaggttgctggtcatgatgatcagcgtgttcttgaagtcgatggtgcggccttcgccgtcgttcagcgtacctttgtcgaacacttggtagaacaggttcaacacttctggatcggctttttctacttcatcaagaagtacaacagaatatggacgctgacgtaccgcttctgtcagcatgccgccttcaccgtaacctacgtaaccaggaggcgaaccgattaggcgagataccgtgtgcttctcttggaactcagacatgttaatggtcgtcataaagcgctcaccaccaaacatttgatcagcgatcgcacgagcagtttcggttttacccacgccacttgggccaactagcaggaatacgcctgtcggtgcgtctggattacccaagccagcttttgcggtttgaatgccttcagacagtgcatcgatagcgtattcttggcctttgatgctttgtgttaggctttctttcagttttagcgtggtttctgcttcgtcttgaagcatcttacccattggaatgccagtccagtctgagataacgtggctgacttcatctggcccaacttcaaagtgaactaatgggttgccattgcggattgcgtctaactgttcttggcatgctgaaatcgcaatgcggaccgcttcttcatccatttctgtatatggagaaacgtcttgttctgaatcttcgtctacaacaagctcttcttcatttactttgccaaagaccagttcatgtaagcgagagcgtagagcaatcatctcttggatctgctctttttctttgtgccattgcacttcttgttctgctagctcttcttttgtcgtttccattgccaattttaggtctggaatgttagcaaggctgtgcttgtcgccagtttgctgcagagcatcacgctcaagcgcttcaagctcgcgctgttgagctgcaagttcttgctgaagagtctcaacagaagcaggaatggcattaaggctgatgttcactcgtgcacatgccgtatctagtacatcgatcgctttgtctggcagttggcgaccagagatgtatcgagcagaaagcgctgccgctgcggtgatagcgtcgtcacgtacgtaaacattgtgagatttctcgtacgccgggcgcagaccacgaatgatcaatgctgcttgctcaggagacggttcgtcgagtttcaccagttggaaacgacgcgcaagagctggatctttctcaaagtactttttgtattccgaccatgttgttgctgcgattgtctttacttcaccacgtgctagggcaggttttaataagttagcagcatcgctaccgcccgcttggttgccgccgccaacaagggtgtgcgcttcatcgatgaacaagatgatcggtgttggcgagtttttcacttcatccaatactgcatttaggcgtttttcgaattcgccttttacgcttgcgccagcttgcagaagacccatgtccaagccgtaaagctcaacacctttaaggttgtctggtacgtcaccttgtacaatcttaagtgccaagccttctactactgcggttttaccaacgcccggctcaccaacggcaatagggttgtttttacggcgacgagcaagaatgtctacaatttggcggatttcttggtcacggcagaaaacaggatcgatttcgcctttgcgtgctttaccagtgaagtctgtcgtgaacttgctcagtgctgaaccatcttcacgagcttgtgttttctctgaagtcgcgacttgcgcctcaatagagtggcttgttagttctgcaaagttacgtttaaggctatcagggtttactgcttcaagaatagaggcgtaaccatgttggccataacgcagagggttgctaacaagagtaaggagtagggcgccagagcggatttgtgtttcggatagatctaaagaagaaaccagccagctttcttgtaaccattcgattaataaagcagaaaaaacaggtttgctgccatttcctttggcgttggtatcgagtgtggatcgaaccgattgtctcaataagttctcagaacaatcaaaatgacttaataagacgtcaaaatcactatttggcctttctaatagactcagtaaataatgctcaatttgaacttcgtttgctttttccgagactgcgagtgctgcggcatcttctaatgctactttagctatcgggtgtagacgctgaattaatgaagaaaggtttatatttatcatagccattttattctttagaggaaaagaggtgagagctattatactaaaagtggttacgccatttttttatcaagatattttaactatcgttttctggtgatttgaatggctatgttagtgggaatatttatcacaagtaattatgctttgctattgtgaattaattattggttaatacgactaaaatgccagtgttattaactttattttgttctttgtatatggttattttaactttttaaaattgaatcaatttattattatctgattccaatttattggttttttatccaataaattgtacgaaataaattaacctataatggggttttattgtaactcattgtttttgctctactttaaaagttggcacggagtttgattataccaagtagtcgcggtgataagcgatttggtgatttaaacaaaacagattttagaaaaggaaatagcgatgccaactccagcatatatgtctatcaacggtgaaactcaaggtcacattactaaagatacatactctgcagattcagtaggtaacacatggcaagaagctcacgttgatgagttcctagtacaagaacttgatcacgtgctaactgttccacgtgatccacaaagtggtcaacctactggtcaacgtgtacaccgtccacttgttgtgactaaagttcaagaccgttcttctccactactatttaacgcactagtgtctggtgaaaaacttcctgagtgtttgattcgtttctaccgtacttcagttcaaggcaagcaagagcactactactcaatcaagctgattgatgcgctattggtagatatccaaactcgtatgaaccactgtcaagatgcagcaacagctgatcgcgttacagaagaagttcttaagtttacttaccgtgcaatcgaagtaactcacgaaaactgtggtacagctggtaacgacgactggcgtgctccacgcgaagcataattgctttttgcgtaaagattcagggccaacaattgttggccctaattcgattatttgtatcaggtaaaagatatggtgaatgacgtagaatttaaatttgaagtgccagggtgtgggcacgagtttcgggtagaaagctttcaagtaaatgaagagctttcgaaaccttttcacatcagtctctcattactttcacttgatccagatatctcttttgattcgttaatacgaaaagcaggtacactgactctttatggacaaggcttaagcgcggcacgcatattcaatggtgtggtgaacgaagttcgttatttgggtacaggccgtcgcttctctcgctatcagttaattttggttccgcaggcttggtttttatcacagcgccaagactgtcgtattttccaacaaaaatcagcgaaagatataattacagaggtgcttgatgatggttcagtgaccgactatcgctttgaattgtctggtatttatccaccaaaggagtacgcactgcaataccgtgagtcagacctacactttgttcagcgtatgatggcagaacacggtatgtggtattatttcgaccacaccgattcaaaccacacaatgatcattgtcgatagtaatgatgccattgctccactggttagctctcctctcaatgcatcttatattgggccgatcgtatatcatgccgatagtgggggtgttgctgaccgtgagcatatctccgatctcgagttagttaaccgtgtgagaacgggtcaggttacttacactgactacaactacgagcagccaaagatcccgcaagagatgactcacgcaggtgatctggaccaagatttaaagcagtttgattatcctggacgttatgttgaccctgtaatggggcaggtgagaaccactgagtggatgtttgagcacattgtcgataatcagcaagtagaagcgagcagtgatgtgatgcgtttggcgtctggttacagctttaacatcagtgaccatcctcgctctgaaatcaaccgtgattacatcatgttatccgtcatgcacacagggcaagacccacaagtgcacgaagacgaagcgagtggcatgccaactacttactacaaccagttcacgtgtattccacgtgatgttgtattcaaagcgcccaagttggcggctccagtagtggatggtccgcaaactgcggttgtagttggtcctgcaggggaggagatttacaccgataaactaggacgaattaaggttcagttccattgggatcgctacggtaacaacgatgaacatgcaagttgctggattcgtgtgagtcaatcaatggctgctccaacgtggggtgcggtttacttgccacgtattggtcatgaagtcgtagtcaccttccttgaaggcgacccagatcgcccattggtgactggcgcagtttataacggtcttcacttccctccgtactcgttgcctgagaacaaaactcgtactacgttccgtacgcaaacgcacaaaggcaccggctacaacgaactgagttttgaagacgaagcgaaccaagaagaagtctacattcatgctcaaaaagacatgtcgaccaaagttctcaacaatcgttaccgagatatcggtcaggatgagttcttaaaggttgcgcgtcatcagacgaacgaagtccatggtgatcataaagaaaccattgatggccacaaaacaacccaagtaaacagcacttttaccgagacagtagagcaagacgttaccgttacatacaacgccaacgaaacccaatatgtgaagaacaattctgatttggaaattggagataaccgcacaaccaagatcggcaagaacgatgatttggatgtgggtgaaaacagtaacttaactgttggtgcatcaaaaagctcggacattggtgccgatgataaccaaacagtgggtggcaacttaaccgtatcggtaaaaggcaatacatcttacaaagccgatggcgcgacgcagatcatcagtggtgacaagattgtactgaaaacaggcggttcttctttagtgatgaacagtgatggttcaatcaagctctcagggtcttcaatcaccattgaaggcagcgataaagtcgtcgtgaaaggtggtaacgtagcgattaactaatgatggaaaaacaaactcttattaaagaaaccatcacgaaacagcaagccgatacaccggcttgcttttcgcgttttggaaccatagtcgccattaacgaatcaacaggcagtgttcgcgttgattttgatggcaacccatttaaccaaccacttaccgcaaggttagggcgtggctttcgccgctctgaattgcaaatggctatcgacaatcgattaaattgtcgaatcgaatttttgaacgacgacttaagcttgccccttgtgacggacatttttttctccattctggatgactctgatgagtttgtattgcgggcgaagaagatggtcattgagacggagcaagaactgatcgtcaaaagtggtgagacagaaactcgctacagcgggcgtgatggccgtattacaactaaagcgaagtacgttacttctcaagcagaaaaagcacaaaaaattcaaggcggaacgatcgcaattaactgatgactatcagtgttaactaattacatcgtgttaactaattgctatcgcgcgctaacaacggataagtagatgcgacaaagaattggtgtcgagcacctaaaggcacccataaccaatcaagaagtcgaaggtgcgttggccaaagctgaacgtgctgttaaagacctttcccaattgccagatacatggctggacttctgcaacgaaaagctggctatcacttctgagagcctcggttttttgatcaggcagcgtgttcaacttcataaacgtggttatccgagccgtgaactggattacctgaaattgatcgaacgccaaattgaagagctcaagaaagtgtacttgtcattctatcgattagctccaggtctgattcatcaattaagaggtcgagaaccagaaatctatgcttggctcacgctacaaaaagagctcggttctgagttcgataacctcttgtgtggattaagtttactcgaagagttagacgctcaaaccgcgatggtcattgttacgcaatcgccagttgaaaacatggatagcgtgatgtcggagctcattgagggcaaagcgaaatccagtacgttttactttgaatacttacgtgtccgccaggcattgagtgttgcgttgattaaacgttggcacaaagccgaaattattcgttctaatatcgcgctacctctactggcattgcaagatgtggaagaggggattgaatggattaatgacaatgcaaatactgaacaatatctgtttgaacgtttgatcactaaacgcgacagaggcacatggttccgtcagtgttttggtatcgagccgagcggtttgccttctgcacaagtgttgacctttgcaaagctattggagatcaaagagtttgaagcgtttgatatatcctcttcattagctcccgttgattttgctctgagtggcgattggaagttagtgccagaaatcatcgagcacttggaaacactggaagaggatgagggcgaaatttggattcaagctttgtatgtggtttacggaaagcttcttcccctgactcctcaaggtgttggtgtcgagtatgaatgggaagagatcgtcgatctgctcaatgagtgggtggaagatgaaaaatatatccaaaacctaccgagccgcttgggctacgcattgagcttcgaatctactttagcagcgatgcaagatgccaacgtcgatgtcttgtttcgagattggctatggcgacaaatttgcattcaatctcgagcctacgtgccttgggacatggcgatgccagtttaccaacaagattggaatttcaataacctaaaggccgcaccttctgcgagtgaacgattcaatttaaggaatagcaatgcagttatgggatattgaagcgtacccagagctatcgattaaaggccgttttcaacgtgacgaaaacggtgatgaagtttgggttgtggttgcgaaacgcacatggcaattcgacggtgaggtttggcacgaattgggtgacagcgaaatcttcgacgatccccagtatttgggcgaagaaggtttctctgcgatcaaggtcgaccaagagttcgcttataccaaaaacaatactgatgtgcttgtctacggcaaagctcgcagctacgcgaaaaagccagtgacataccaagaatgtcgtgtgctgattgatggtcatatcgataaaactctcgccgttcacggtgaacgcgtatgggttgagcatggcggcagcgtcacattgagcaaacctgtgccttttattgaaaaggatatcgattacacctgcgcaattggtggtgatttacgtaaccgaattggcggtggtgtggcggattcgaacaaagaactgcttgagcagaaagtgccttctgtgttctatccaaaagaagaatggagctctacatcgaaaaatctaagagtggctggctttggacctgttcctccattctttgaggctcgccaaacattggctggtacgtttgacgaagaatggattgagaaccgcaagcctatgcttccattggattttgatcgacgtttctttcaaagtgcgcctgctgatcaacaatgcaaaggctttcttaaaggtggggagcgcttgatgatgagtggtttctgtcatgatgacacgatttcattccgcattcccaaagaaaagtaccgcgcaatcgcgacgttcaaagacggcagttatccagttgacatggcgatgtacactttatttgttgatgctgaaaagaaaaccatatcaattagttacacagctgcatttccttgccagagcaaagagcatttattggtgtcgacgtctattacgaaagtggaagaggtaagcgaacatgcataactcaacaggtcgttacgtgctgggaatggaagtgatgacgcctactggcatgaacttggatattgctaccagtgtagcaaatgctgacttagccagattcgaccaagtagttggcgaggacggaattgagcgtttcacctttgccaaaattgaatacacaaaagaaagtgatttgttcgaaaaaacctgtgagttaacggcaaatttattggattcgcttttgacacagctgcctcgttctttgaagccgattccattacttattgctgtgcctacgacaatttcattagtgaagatccaagaatggcttggtgaaagtgactattctgactttctttctgtggtcgaagcagttcacgatagtggccctagcttcgtgctacaagccatgaagtcgttggataaatacgatgccatgatgtgtattagcgtcgattctatggtcaacagaatacaagaactcatcgacgataccatggtaatgagcaccaataatccatggggtgttattccaagtgagggtggcgcgggcctcattttttgccgaagaaataccgtagaaacactcaaacttaagcctttagcgcaacttggttatatcgatacagaactgaacacttcagaccgaagaggtatgtatcgtcttgtacagcgtgcgagtaaaaagcttactgctttcggtgaagtgtattctgatatgacgaacctgcgcgcgcacagtgaagattacggtttcgcgctgggtgccaaagctgagcgatttattaacccagaacaacctttgcttatcaacgagttatgggggacgatgggaaactgttcatctcttgctcttggagcatttgcggttaaatatcatcactttaaccagcccgtaacgctgctgatgtttgacttcgggggtgataaagcgctacttcaattactagcgtgctaatcattctttgtcgaaatatataatgaaaatgaagtcatattacgttgttgaaattttaatttctaaaacaacgtaattcaggtagtgaaagcgtaagtcaaagagtctaataacttagtaattataactaaaataataggcttactgttcgttttaaatactacccttgaaacaaggatgtttcagcaaatattatttcggtctaataatatttgttccataaaagagtgcaaagtgtctgatattcagggtgcagccaaaggatacattgatcagttggttgttaaagccattgagaaaaagaaactgaacgagcaaagattggcgcagaaaaataagccaaagcttgagtacgttcttttttcacaatttgatgtattagacacgttgccatctaaaaatggtagaacgacggtttaccacctcgcaaaaaaaggccaaccagaaaaacaaatttgctgcaaagtcgtcaatgaagacgccccagaagtcgcaaacaaaatgttggcgaatgaggcaagcagattagaactttctcagcatccaagtgttgcggactttattaagttcggcaacgagtttgaacgaccatatttaatgtatgagtggattcaaggcgaatcactagcggaaaaaatggaacgctattcttccaaaggctttcgtcatgatcatattgcatggttagtttatcaactcgcaggtgcgctagaatttatgcacactcgaggtatttgccatcttgatatcaaaccgtcgaatgttctggttagtgaaggcgactacgtcaagcttatcgactttggtgcagctcgttatatcggcgagattgaatctcacgcggaagcgagtttaagttacgcgtcgccactttatctcgagacgggaacaactgagccacaagacgacgtttattctcttgcattgcttagcggtcacttatttttaggggcgatttttggtgatgcttggcacaagcaattaaaagatcgcaaacgtcctgcattaattccgaaccatatttggaagttgctcaagcaagtgataaataacccaagagggcatggctacacagctatttcttttgctcagcaacttgcccgtattgatactcaatccatcgactctcattctaatgcgcccatttttagtagtttgcgtaatgcggatctactgctgactcattgtagacctcaagacaaagcgggctatggccgctttaaagtgcttgaagcgagcttggtattgagtgttatagctattacgggtaattacctttatcaacaaagccagccagaatggaaagcgattgtaaaatcagaacctacgatttctaaaaccgaaacaccgttggtgagtaatatcaaaccagcgcaaacggcgtcttttttggctcaacctccttggattattgaaaaggctcttaatgatatggaaaaagacgttgtgtcgacagcaccatatcgagaagcttatcaagtacagcaaatgaaactatctgcgctgtataaacaaaaccaagcacaattaatgtcttaccaagctttggcagatgacttgccaaaagagttggtggatttacgcaaagagctagtcttattacgagaaaaactcgttgatgatggtgcgttgtttccatacgcagataagactctgactcaagtaatggcagggttaaacacagcgtcagtaaactcgatgaaaatctccgctgttgctggaagcaaagatgaacgaatcgccaatttgatcttggatggtgaagcgaagttggctgatgaagagatgaaagcggcttggttattaaatcaatcggaggcttacttttacagccaagtgctgccaaacaaggtgatggcgaacatcaacgactctattgaacagcatgcaaaagatcattactacaccaaagccatagaagaggcgaaagccgctaaaacgtactttggaaatgcgccagaattaaacaaaaaggtgaaagacctgatcgttgctcgcagcgaatacattctgtttagcactgtgacggagcaatcggtgtttgataaacaaaaacttcacgattctctggctgaccttgagcgaaatgcgccgacaaagtttggtgaaattactgatctgcttgaaagcatggccacagactctatcgataaaagtcatcaactttctcgtcctgcacgtggtgcaattgcaattgaacgcgccttgagagattacctagcagaggagagaagttaatgtctgtgattgaaattgaacgtttacttgcgcctatctctgacgtaaatcctgtcggtgaagatgctcgttacgagttttgttatgaaatgatggaatccgaagtgaagaagttcggatccttgtttggcgaaacggtagattggagtgtcgtaaaaacgcacgccactgaagttcttgaacaccacagcaaagatttaaaagcaatttgttaccttgttcgcgcgcttacagaagagtttggtctgcaaggtttcgagcaaggtctaaagctgcttagtgaagcgttaaatcgctttggcgttgagctgtacccgtcgcggaaacggggtagagatggtgcggtagagtggttgaaccatcagtttaaattggtgagcagcaggtttgcagaaagcgctcagtcatgggatttggtttcaggttgtatttcgactatcgaagaagtccaacgtcagtacgacgatatttaccaagactccgaagcggacttctttgaaattcgtagtcaactcaatgcgctatctcaacaagcggctgtcgatgagcaaagcgatggtgcaatggctaccatcgaacaaacagctccgctaacggcttcacagcctgtagcagtagaaactccggcacaaagtgaagttcaaaccgttgtagcaaaagcggttccgtcggctcctaaacctgcaccagttaagaaaacagtaacaaaagaagtcgatgtcgatacggatttttcatcgccgaccgcttctaaacgtacacttaagaaagttgcggaagttatgattcatgcgaatccaagtgagccgctagcttaccgaatttatcgtcacctaacttgggatgatattgatggtcttcctgatcatcaaagcaatgaaacgccattgagtttggctgtgtcgtctgatcagcaggctgagtaccgcgataaagcctctcaagaaagcgacattgatacagttaagcgtttggagcgcacgttaaccgatgctccgttttggctgacgggtcactactttgtttattcgatgttaaacaatctaggctttaatgacgcagcgttcgctgtgaagcaagaagtgaagcgttttgttgattcattagagggcattgaactcctcacatttaagaattctataccatttgcagatgaagcaacgttgagctggttatcgactcaagctgcaacttcttcaagtgctcaatctgtcgttcaaacgattgtcatcacagatgaagactcactgccaatggaagatatcaccctagccaacttaggggagtatgccgcagaactcgcacacaaactcgaactggatttctcgggacgtgggcagtttatgctgcatttacaattgatagcagcttaccagtctgttggattatacccgttgtgtttgccttaccttgaaaaagcttgggaagtgcaacaagcgtttaatctcgcgagctgggaaccccacttatcgtcacagttagaagacctgattcgaaaaacactgcatcagttattcagaaacaaggatcttttgcctgaacagtacgaaaagtggaaagcaatttatgactaattaaagagttagataaggaactaattatgtcacgtgacggctcggtggctcctaaagagcgaattaatatccgttatgttccagcaactggtgatacgcaagaagacgttgagctgccgctgagcatgatggtggttggtgactttactgcacgtgcagatgagacgccaatcgaagaacgtaccccaattaacatcgataaagacaactttaatgacgtgttagaaggcatgtctccaaacgttaaagtaaatgttgaaaaccgcttatctgatgaagaaggcgcacagatcggtgttgacctaacgttccaaaatatgaaggacttctctccggaagcgatcgcgaaaagtgtacctgaacttaacagtcttcttgagcttcgcgaagcattggttgcactaaaaggtcctctaggtaacgtacctgcgttccgtaagaagattgcatcggttcttcaagacgaagaagcaagaaagaaactgttggatgaactaagcattggcaacgaccaagacgcgaaagaagagtaagggatatcatgtctgcagaagcacaagctccagaacaagaagcagccttagttgaatcaggctcacttctggatagcattcttaacgaaactcgtttaaagccaagtgatgaaggttttgatgttgctaaacgtggcgtagaagcattcattagtgagctactgagcagctcgaatacagagaaagtagaccaatctttggttgacttaatgatctctgaaatcgaccaaaaactgtctaagcaagttgatgcgattcttcacaacgaagaagttcaagcgattgagtcaacttggcgtggcctgaagtatcttgttgatcacactgatttccgtgagaacattcaaatcgaactaatttcagcgaagaaagacgaagttcttgatgattttgaagatgcaccggaagtggttaagtctggcctttacaagcagatctacactcgcgaatacggccaattcggtggtaaacctgtaggtgcggtaatctgtgactacaacatgtcagcgtcttctccagacattaagctgatggaatacatggcaaatgtgggcgcgatgtctcatgcacctttcataacgtcagcatcggctaagttctttggtctggatagctacgaagagctaccaaacctaaaagatttgaagtctgttttcgaaggtccacaatacacgaaatggcgtggcttacgtgaacacgaagatgctcgttacctaggtctatgtacgtctcgctttatgttacgtacaccttactctgtcgaagataacccaattaaggcgtttgactacgacgagcacgtaacagatagccatgataactacctatggggtaactcagcgtacgcgatggcttctaagatcagtgagtcatttgctaagtaccgttggtgtccaaatatcattggtcctcaaagtggcggttcggttcatgacttgccagtttataactttgaagcaatgggccaaatcgaaacgaagatcccaacagaaatcttggtatctgaccgtcgtgaatacgagctagccgaagagggctttatcgctctaacgatgcgcaaaggttctgacaacgcggcgttcttctctgcaaactctgttcaaaaaccaaaagtgtttgcaaacacgccagaaggcaaacaagcagagatgaactacaagctgggtactcagcttccatacatgttcattatcaaccgtctagcgcactacatcaaagtgctacaacgtgagcaaatcggttcatggaaagagcgttctgacctagaaatcgaattgaacaagtggatccgtcaatacgtttctgaccaagagaacccaccagcagaagtccgtggtcgtcgtccactgcgtgctgctaaggttgaggtatcagacgtagaaggcgatccaggctggtacaaagtatcaatgtctgtacgtcctcacttcaagtacatgggcgcaagtttcgacttgtctctagttggtaaactagaccaataacccattattgaaaccatcaagcagccattcttcactggctgcttttttcgtaggaccgaacatggaaaaaggttatcgtcttttagagcggattgaactaggtgagccaaaaaactgctacgagaaagtggtctcccacaagcatttgattgaatctattcatttgcatcttgcagatttgctgaatacgcattctggtaatgcgatgatcgatagtgaatatggtttgcccgattttaatgacgttctgtctaataatacgaatctcgttcgccacatccaaaagaacatcacagcgaccatagaacgctttgaacctcggctacttaacgtcgaagttcactatcgtgaagaccaccacaatccattacagctcggttttggtattcgcggagaagtttcccataacggcggtaaagttccaatgtcgatcgatgtctacatgggaaccgacggccaattcaacgtttagtaggtgtcacttgagtaatagcaaatactttcaagatgaactcacatatttgcgcgaatcgggtagtgaatttgccaaatatcatccgaagttaactcactttctctctgaaggaacattcgacccagatgtagagcgattgttggaaggtttcgcttttctgactggacgtattcgagagaagatcgatgacgaactgccagaactgacgcaatctctgatgactttgctgtggccgcattacatgcgttcaattccatctttgtgcatcagtgagctaaaaccccatacgggaagtgttactgagaaaacggtggtaaaacgtggcgcagaaatggccagtgagcaagtggagggcacgcaatgtcttttccgcacttgctacgatgtgaatctgtacccaatcacgatcaccaacattgaacaaaccaacagccgcaccagttcagcgattgatgtgacgttgtcgacggagcatggcctagagctgtctcgaattggtttagatacgttgcgtttgcacttgcatggtgagattcatattacccgcacagtcttcctctggctgttccgttacttggattacgtggagttggatgtcggtggcggctataaacatcgtttagggccagaatatgttaagccggtagggcatgaagaagacgaagcgcttctgccttacagcaaaaattcattcgcgggttatcgtttgctgcaagaattattctctttgccagacaagtttatgttcttcgacattacaggtttggagtggctaaagggtattccacaacgaagcacagtgaaaatcaaatttcactttaaacgcgcattgccgtcagaggttgtacttaaagataagcacctaaggttgcactgcacgccggcggtgaatttgtttgaaaaagatggcgacccaattcgtttggagcaccgtcgtaacgaatacaaagtccgtcctcaaagtaatacacaagagcactatgaagtgtattccattgagcaagttgagagttggagcaaagacgagcgtcgccgtaaaccgttaatcgaattcgaatcgttcgagcatcaaatcaaccaacgagacaagcgagaattctacaagtcgaaggtaggagagcgtgttagtggaagaggcttagagcgctatatctcgttccacactcataatggtgatattgcggatttaggtacggaaaccgtactcatgaaactgcaatgcagtaacgctgatttggccgaacgtctttcggtcggcgacattacttacgcgacccacaaatcgccaacgtatgcgacatttaaaaacatcaccaagccaacacaatcggtaagcccacaggtaaacggcgagcttcaatggcagttgattgccaacatgtcgctcaattatttgtctttggcaaacattgatgtattaaaagtgctgctctcgacctatgactttcattctcgtgttgacagacaagctcaccgagcatcgattcaccgactcgatggcattgtatcgtcagagatgaaaccaattgaccgtgtattccgtggcgtgtctgtgcgtggtaaccagttcaaactagtaaccaactctaagttcttcgtaaatgaaggcgatatgttcctgatggcaacggtactgaacgaattcatccgtttgtattcgagtgtaaactcctttacggagctagaagtatttgatgaggcaaccggcgaagtatacaactggaaaagcttaatcgggcagcagacgattctatgattaatacactttctgaaaaatcgaatgagtttagtttttatcaagcggttcttttgctcgagaagcactaccagtttgaaccaaactctaactttgtcgccgtgggagagaacaaatatttccaccaggagcgaattgagttcagcgtgtcgcctaatctatgcttccctaagagtgatatggaatttatcgttcatatggaacgaaatgggcagcagtattcacgagtcgagacgaacttccttggtttgcatggttcgagctctccattgcctgtctcttatacggagaaacttactgggcgtgaacctgatgacaacccggtaaaagatttctttgatttcttccataatcgctacaccagtttactctatcgtgtctggaagaagtaccgttatcatgtacagtaccaaagtggtgcgacagacgctttttctggacggatgctgcacttagctggtttatcaggtgttatgcaagattgtggtgttgcagagttagaccgtgcaaaagtcctgtcttacgttaatcaactgtcgacacgtacacgttctcctaagctaatttcaggtatcgtttcacattatttctcacttcctagcgttcgcatcgaagaatgggtttatcgccgtgtggagattgcagacagtcaacgaaataaactcaatcgagccaattgcatattaggtcaaagctttcatttagggcagagtatcgcagatttaaatggtaagtttaatctttgcattgatgacattgacttcgagacatttaagcagttttcttacgaaggtgagttgcataaaacgttggttggtttaatgcgattcatcttgcgtgacccgatgtcttgggatctcaaactgacagttaatctcgacagcattccaaataaccaacttggcaatggagaagggaacaacttagggcagacattttggttaggcaatcccacagatgaagatgtgaagatcagagtaattggtagcatttagatgctgtaggccaaaaagccgctcaatgagcggctttttgatgtgtgtgagattgggaaaactctatcgactgatcggctgatgaagtacaaccgtagtgtctagtttttgtatcccattaaaagactcaatttcgttgcaaaggtggtgaatagactgaaggtctggtgcttcaatcaaagcaataatatcgaagctgccgccaactacgctcgtaagacgtacttcaggaatttcctttagggcgttagttacttcgtttttctttcctttttcacatgagataagtaaatagctagcagcttttctcccgtcactttcaatccgaatttgtgcggtatagccttttataataccggttttttccaacctattgatcctttctgcgacagcagtacgggacaaaccgatatttctagagagatttgcaatagactcacgcccgtttgttaacaggctggaaagtattttgcgatctaacttatctaagccttgtaaagtcatattagaaatcatgtgttctgcctattctggttattatattgttatcaaatcgactctgggagcgtaaagttgtacaactcagccaaagtgaaagggtttttttgtgtcaagggagttatacggaggctcaaatccctccctgatacttttatgtcactaacgaagctaccgttatgagtgtttgtcagggtcgaatcacctagcaatctatatatcgcccattgaccactcttatttaaaacgtgttgtttaccttcgtctgtcacatcttggatcgtgatactaatgttgaagtcgccgttttgcggtggccactccatttcacgaattcggctcggaccgtgatagtagttgatatcagtgtcagagattttcaaactggctctgatagcacttgagtcaagatctaatactttcgcactgaatggaatataaactcggttagtactcttattgatcagcgtgtcgcggataacgttgtaattgcgcaattgcaccagtaaggctgaagataatggcattgtttcaccatccacctgttttggtacccacaagttggtatcgtagaacggtgcgaagtttttctgaataaatgtgtctaacaatccacctgaagcaaacaacagttcgaagtcttcgatagaaatctcctctgttgctgtgagatcaaatgggtatttatcaagtcctatctctttaaatttggtgtaaatttcactataccattgggtttgaatgccttttgctgattcggtgatcattaccgaccagctttgctttacgacatcaagtaaccaactgcgtactggctctggtgacttctgagcaatctgtttaagtttaattagcggatcggcttcagagctgctcattcggtgttgagcggctgcaagtgctgccatttgtggatcgggagcatccgcaatatctttcatgtaggtgcgaactcggcttaaagctgcgatagtttcgtcccatggagtaggggagttaggagtctcgcttacttgaagctgatttagaagatggaacgcttgttctacacgcttcatcaacaagtaatccggctgaaccacttcttcaacctcttctgacgcagagttcgccacttctgccagttttgggtctactttcgagatgagtacgtttttttctcctaccggtgaaaactgagtatttgcatagacctgctttaatacagtcgtcattggtgaagatggtccagagattaggtcgatagcatttgttagatcacccacattgctataactctgaatttttaactcactgagagcgttgcgccagtagttgatataatcatcggtgtactttttacgtacttgatttttaaacgattcttgctcttctttggttgggataacatagctggacaatcctagcacccagttatcactgatcacttgtttagacatcaagtcgacgcgagggcgatagaaggtactaaagccagtggaggtatagatcttatcgattactagtcggttgtcatctaacggctcgttaaatacgttgttaaattcaaagccaactgcacgttgtagatcgagtgtgccaagatcgatagtatctgcttggtttaaaatgcctgcgtaaacaagatccacatttgaatttgccaacaatgagcgacgagttgctcgaacgacatccatgttgatatctacaggtgagaattgagtgcggaagtaggcgtccaagtatcgcatagcttgatttaccacatccggcttatcgctcaagtctgtcagtgtttgcaatgtttggcgtcgtaaaaactgaatatctcgcttggtcggatccttcaacatcaagtagccttttaataacggcaacgttgctgtgacatcatgttggttgtgtgtcagttcaaaacggtagccttgctcaataacaggcataagttgttgagcaacctctttaatcaatgcttcatacgctaattttgtactttcgtttaacctgcttatatttagcggtattacttcttcatctaatgctcgtgatccttctaaccaagcattgtatgagggttggatagcacgagtagcactctgcaataactggttgaagcttgcttcttgttctgtttgaactttttcgtcgatattcagcaattggttaataacacgcaagttactgtctagtgttgtgacaaaaaaataagtaccgcctgccaataagacaacggatgccagcattgccaaatggctttgacgctgaatcatgcgtaggtaagtcttgttctcgcctgcgaagtcactctctgggaggatttgagagtcaagcaagaagcgagaaaagtacggtgtttcagttagttgcgtgtgctcagatgcgataattggtaaattgaagtaattactgcagcttttggctaacaagttgtacttgcgtccaccttgcaaagaagaacagaagaagatttcgcgaatatccaacgagtaaacgccattgtttgtatcgcaaagacgttcaagcacgtagctgatttcacgttgacagagttcaaactgttttgggaaggcaaggatggcttgcttttcgttgatatctgttgagtttgccgatgcgtctaaagcgttgctttctagcacttttaccagattatggaagctatctaggtagtattcggtaagtacacctttcgcttccttcaatggaatggacagaaattcaatacgtgtttttagtgaactagagtgaacgtattctttaaagccatctagtttgtcgagtttcgtcatcatcaaataaacaggtagtgcagacgaggtcttctcagtgatagagttcaaacgctgcaatagcgctgtcatgatgtaatctttctgctctggctcagatacaatcatgaattcgaaatcaacaaagaataagcaacctgaaaagggttttctcggacgatgacgaatcacctcatcaataaaagtattccagagagaagcatcactgcttgaaacttcctggaagatgaggcgttgatctggcttaacataaacggcaccatcagattcataccattcgaagaaatcgttttttgctgtgcgagaacggtccatcggcttaattgcgctggaattaaacaaaaactgacgtcgaccggcccctttattaccaaccatcagatagattggctttttgcccgcattagaaagaagagggcggatcaccattgaaagagcttcttcttccgtctctaaacgttgaacttttgagctgttttttttcttaagccatagggaaagctggaatgtaaaataaaaaccagtaataactcctaaaccaacccatagataaagcttatctgcgtcttcaagtatgaataaatataccgctgcaaatatcgcggctaataccaaagccactatggtagaaaccacgactggatttcttagtattttttgtttgaacataagcctgtaacttctttacttttatttattgtctttttctgcagcttgtagctggttgattttatataggtaagcgttcagcaaaaggagtgatcgttcaatttcctttctctgagagttcttttctgcatattcaattcgcccaagaaggggaaatagcgaattgctgtactcatatgctttcttagcgtccttgtctgcactttgttttgcaatgttggcaaagaccgttctagcatatgcaaaacgtttgtatatagcctcaaattcatcttcaaaataagaacgttggcctaaaaaattatcgcgaagttgttgtgcagaggatgagtctggatagagcttggtcaggtcattcgttaactgttcaatggtattgacctggctcactttgactggatctaatgtccaacctgacatcaaagtatgaagacgctcaaccgtgacagagcgcaagtcatcttggtaatttttgttttcgacactgagcgagttaacaatcgtcactaattcagtaagtgatgctgacccaattctttccttgagtattgtgtattgatgctgtttgtatatgaagtgagacgtaaaagctatgatcagcaatccgatcattgtatagacaatgaatcggcttgatttggcctcttcttccactttaggctgctctactttcttttgaacaggtctattgggcggtgctatttcgactcggatgcctgctgaaggaggatttgacgcaggttttgtttgtgaggttttctcttgtttcgccttttcttctttaatctcttcaatccactgatttaggggacgacgaatacgtccaaaagaaggtgccttaataccatagtgaaggcgaagttcctcttcgatacgtagacaaagctgatgagccgcttcaattagggggagttcttccggcaggactcggtgtttcttcattcgtttttcagtgtactcaaccatccattcaatcgcactaatacgggcattagcttttgtgtactcaggataggcggagaaccaataaacaacacacaagtcgagcacagaatttaagccctcaactaggccttttagtccgtcattattaatggctgccacagtaaagtaacagctacagcgaaagtctttgcctttcgttctaaggatttcttttgatagtgtctgaactgtcttccatgacacacggcctgttactttgttcaagccatttatttgacgttttatttcatcaaagtcgtctaagccatttggattggtaccgcttggattactttcatcgatcggtcttcttgtgaagtcgctgaaaaacataatgttaagttccaactgagttgataataaaataataaagatagatttttttattgtattcgcctgctatgccaaatgacatgcaatgcattttttctgattttatagataaaaaaagatttttagacaataaaaagccgataaatctaattaaaatccattttgtgtgatctttgttcctaatattaatatcaattggctaatttgcgtgtaggaaaaatcctatttttcattcattttgttagttttttatgatgtaaatgaacattttaataaacttaatattaggtgtttttttattatcaattgctgatatacggtctttgcttattcaattttctagaataatgcggctatttatcaaggttctataatgcgcctgttaatggttgaaatgcctattttgatagttaaagtgtgctttgttgtggtcgctttattttatatcttgaaaaagagagtgtaattgatatcgtccttcctccaaataataaaaaatagattttattggggaaatcaatgtcaaaatctagtcaagttggactcatttatgccatgtgttttttttctacttttgcggtggcaaaaggggagcgatatggtgcttcagtcaatatgatcactgtagaaagtattgactatgacgaaggctctagtggtgacgtagatgctttacgatacggtcttgttcatactcgcccaatcgatgaaaataacaaccgttggcgctggtggcttgggttgaattatctcgcggaagatgtcaatgccccttcaaacggcctttatcaagaagttacaaattacgagtttcgtgttgttcctcaatacgctctgagctcgtggagcatatttactccttatgtcggtgctggcttatcgtttggttactcgcagtattcaaaccgttggaaagtagaccaagatggctaccgttacgaaagtgtcgaagatacagatcagtttgagttgggtgcagtcgttacatttggtacggccatcaaattaggcagtaaccctgatgcgcacattcaaattattccccaagcttcctatattcttccagtcgttaatgacggaattggtggagttgagctttctgtttctctactcttttaaggtttagcaatgcaaacacatcagctagtcctatttatttctaaatgcccagaggaatatacgggcgcaaagcatattgaaatgccagaaggtggtggctcagttggacgagcgcctagctgcactctgtctttaacggaccataaccgttttatctctggtacacactgtttgataagtgtatacggtgacactttctacattagcgatgtgtcgacaaatggcactatggttaatggtaataagattctaaagaatcaaccgatatctattgttgaaggtgacgttgtttcattagggcagtacgagattggcgttgcattagagcatatatccgctgttcaagatattgccgccgatattgccccagaaagagtatcgaatgatccactcgtcaatctcggtgaggcggtcgtagaagaggaagaaaaagtcggtgcattagaagacttgttcatggagaccaagcaagatggtgttaatactgacgatcctattgcgcatctaaagttttcgatgcagcgtgaagacgactatcttatccgcgatgttgaaaagcctgagcctgcaccggctaagacaatcgaaaatacgcgacaagtcgttgatgacagtttcagtatccattctgaattcgatattccaaatttaatccctgaagattggctgggaggcgttgccacgagtaaagaatctgttgaaccagcggcagagcaagctaagcaagccgcgttcattccagaagattttgcgcacacgaagcccgtttcgcctcaggctagtgtggttcaaccagacgttaaccaacctcaacagcctcaatatgaagcgccacttagtcacgcgacgtcaaatgttactcaaagagcagagcctgttagccagaaatgggaagaggtaacacaagcctttgttccatcagcacaacctactcagccagaaacgaaagcgaaagaggcccaacaaggttttgtaagcaacgagtctctagcgattgagagtgtggctcattcttcagatatcggtaaagcgttttatgaaggcttgggtattagtaaccctgatctgatcagtaacgaagctctgctgtttaaacaaatgggggcatgtcttcgtttgtgtatcgataacttgcaaaaagacttgcatgaagttgagtcacttaaaggtgaacagggtttaagcgaagcggattcaaacttagcggaactgatgttaacgctaaatagtcagaacttgctgtctccgaatgaattggtggagcaaatgttggatgagctcaacgatcatcaaattatttttaacaaggcactcaatgagttgctaattgaacaatcagagacgaatgaccctgtcatgtttgcaaacgacgttgcaagtaaatccatgttcaccaccaaatcaaagttatggggtgagtatcttgagttctacgccaataaccgtcgtcaaatgaatgagacgtcattgaaaggtttgatcaagaaaaactacaccaaagcgattaagggaagccatgcgtagtattgcaatattgctcggatgcgccttgctatccggttgtgccatgtgggatcaatttaaagaatcaacaggcatcacgcctgaaacgtcctcgattgagttggtgattgaggcgtcttcggttctaaatgttcgtgaaggcggacaatcctctccggttattttgcgagttcacgaactgacgtcaccagtgttgtttcgtagcttagacttcttcgcattgtttgagaacgataaagcatcactgggtgatgaatacatcaaacgttatgaataccaaatgcagcccggtgaaaagatccacgaatttttggaactcgacccagcaacccgtgcggttggattctcagtcgcgtttcgcgatattgacggttcttcttggcgtaaagtcgaagtgattgaagagaagagcgagtactacataaaactcaagttagaaggcagtgagttaatttctgacaacactcgcggcattgaacaagtttatttttaaggaataaaagcatgtctttatacaatccagttgtttggcaagatggaatgttcatgaagccgcagcactttcagcaactcgatcgttcacaaagtaaactgtcgagcatgctgagcgcaaatgcatcacctttgcattggggcattaagcgcttagagattaactcgcaactactggcgttgggcaaaattggtatcactcgcgctgaaggtattcttcaagacagaacaccatttgagctgccgctgctggctgaacttccagaagtgaaagacgttgatccttcgattgctgacaaagtggtttacctttgttgtccgttaccttctgagcgctctgaactattcggcacgaagaaagacggcgcacgttacacgctggaatctcaagaggcggttgacgcttgttatgattcagaagacatggcaaatatcgtagtcggtaagcttaacttctgcttgatgtatgaccatgaagataagagtgcttacacatcaattccaattctgaagatctcagaagtgaagccagacggcagtgttatcttggatgaaagctttatcccgacttgtatcgatattcatgcctctaccgtgctaaacaagttcgcaaccgagtttgcctctatgctgaaacaccgtgcagaatccatcgttcagcgcttaggtgtggttgaccagcaaggcgtatcttcagtttctgatttcatgctgctgcaagctctaaaccgatacgagccgctattctggcactttgcgagcgcagaaggggttcatcctgaatcgttttatcgcattcttcttcaagcggaaggtgagctttcgactttgtgttcagcatctcgtcgaccaatcgagttcaccaagtacaaccatggcgacctaaccagctgtctatcgcgtacattagatagtgcgaagatgacactaagtgtgatgtctgagcagcgtgcaattccactgacgttgaaagaccaaaactacggtattagaacggcagcgattccagacagtaagattatcgacaccacgacctttattcttgcggttaaagccgacgttactttggatgttcttcacacccagtttgttagtcaaacgaagatcggttctatcgataacattcgtgacttgattaacctgcaactgccgggcatcgagattaaaccaatgccagtcgtacctcgcgcattgccttaccacgcaggttacacctacttcgaactggataagactggcgaagagtgggcagcactgaaaaacacggctgcaattgcggttcacgtagccggcgactttgctaacttgtcgctgcaactatgggcagttcgtctatgagttacgcagaaactgacgttactgtagtcttattccaaccagagccaggcaaaccgatggaggtgatgccagcgcctgacttgtctcgtaatatcgcggtacaagacctaaacatcgaaaacatgggcatcaacccgttggtcgaccagttctcatggttgatcgccagtttgtcttgtatgtcttcgattccttggcttgatgacccaatgccgtttcgtgaacaagtagctcgtgagataagaaaaggcgagcgtaagctcaatgaaatggaactggatcgcgcatccattttggtcatccgctactgtttgtgtgctgcgattgatgagtcggtctgccgacaagagtggggcgctaacagccattggagccagaacagtttgctgtcagagttccacaatgaaacatctggcggtgataagttcttcgttattctcgagcgattaaaagcagacccgcgcaaataccgtcacgttatcgagtttctgtatttgttgcttcaacttggctttcagggcaaatacggccgtgaagagcgcggtaacgaaaagctagcagaaatcggcaatacaatttatcgcttagtacgtgatgaacgcttagcagagcaagagaaagtctctctcgttaaccttaaggccaaataccttaagaagccgttaaaaagggtgatttcacccaaactgattttaggcatcagtgcgattaccttcgcaatcatgtatgccgcgacttacatcgtcatagatttaaagtttcagcaattacttgaagtgtatcgataaacagaagatctcgaaatgagatcttctgccagttttaaggtttattgtgaatgtcactagcgatttagtgaaattcactagtcatttagagggattgcaatatgcgatctcttttattattcaatcaacaaaattcaaatggattggttatgggcgtaacagttggtgcaaatggactttcgatagttcataaaggctcaggtggtgaagcgaatgcaacgctcccagatgtttgtttaacgaaggtcggtaagccgatcgttcctatcccttatggcaacaatgccaaatcagcagatcttgctggtggcactacgactatctctatggatggtggcaacagtgtcgcaattaaaggcagtacgttttcaaaaagtaccggtgatgcgggcggcgataaaaaaggcgtcgcatccggcacgatagaagccgaagcaaaattcatttcagcctcaccaacggttaagtttgaaggcaagggcgtatgtcgcctgtctgatcaaatgaccatgaacaaagccaacactatgtgtttgggcggcgcacaaaacccatcggtttctgttacggaagcagatgatggaacatttaccatagacattgattatcgatacccagatggagacccagtatacaaagccgatttcaagattattgacagtactggtacagaacatgttggcactttaagcgacaaaggtaaggggacgattacgggcttaccagcaggttctatcagtttattcctgggtgaggataagagagatatagagccaatccaaaagccgcaaccaaacaaggattatcttgaactgccggggcctttggacttagtagagtttgctggacgtggcttggttgaatattgggatgctgttgaaatgccgagtgattatagtcaatgggcttggggagcgatcatgggggacttcaatcaagatcgatctgctggccaaattgcttttgatagtgctgttacagctataccattcatcgatcaaattggggacggtcgagacattagtgccaacctttatgctttttataatgcagacgacataagagaagataaagaaaagtacacagatctcgttatcactctagttggctttattcccaccgcaggtagcttgataaaagggttatttaaggaacttaaaatcttgggcaaagccgctgatttagatatgattgctgctttccttagagccggagcaaaaggtgatgtggtcaaatggatgcaaagccttgatatgtccgtcatcaaaaatgatatatatggtcaactagatgatattactaaacaagtaaaaacgttaatggatacacttgagcaagagagccgtaaaagagggtatactgttgttgctgacgcctataaaaggtgtgtggaacagctagatgacttttttaagaaagctgacggaccaatatctaaagtcttaagcgattttgatgataggctcaagatgatcctgcctgaggcacctttagttacatcagggacttcctttagtatttctgcagggacggctcaaggaggaggcaaagctgaggtaattaagactcgtaataagacgaaaaagaagactgatacgtgtccgttgtgtaataaaaagattggaaaagggaagaaagagtgcgaaggggcgaaagtcggtagtatcagcaaagcagttatggacgatggttcctctcaaaaattatggaaagaattcgaaatacgaaatggttggcgagacaagacagagcacccttggtggcacggggcgggagcagtgcaggctcaccatcttattcctaaaaatgcattcaaactaaaaaatgcaaagagtaagcaaactaaagaaaaaatgatgtttcttagacgaattgctcagacgtgcgcttacaacatcgacttttggaaaaatggtattggccttcctaataagaaagaaacagcgtgttttttgaggaaacctcgacatgcaggcggtcacgatcgtaatgaatttaactacacgaacgaatgtgtaaagcttgttcatgacaagttaagctcagatatcaagaaaagaacaaaagaaggtagttgttccaagacaacaaactcttcgttgattatgaagttcaatgaatcaagcaaaaatatttttgataatattgctgactttatctggtttattactcgtgatggaaaaaactatgatcctaaagtagaaccttacgtgggctgtagttcaaattgcaaaaaaatacacccgattattaatataaacactaagcgacaaattgaaaactatgacttagagattggaaaataatgatttcagatattaatgaagagtattatgtacttgtgtgtgggtctggtaaagtaccttctccttatgtttcttgtagtaaaagccactatttgattttccaagaacctatgtcacttaaaatgccaatccggatagagtcagcagctgaagcccaagagaaagttccattaatcgaagctatggctgccccgagtgatcctgtactttcaggtagaataacacaagtcctcaactactttgatctttataaagttcaattgctcccagctatatatacccataatgatgatagcgagcactcttattcagtcttagttgttgataacgatattgatgcttacgattttgataacggcctatatattgatcgtttggatgatggtgaggttggaaacccaagaaactgtcgacttgaccccgaaaaactttcaaaaatcccgttagaaaaacgttatatttttaaaatacggggcatgatggattatttagtccacaaaacaattgccgaatctctagtcgcattgaatgcaagtggacttcacttagttccagtattggaatgggacattggttttggaatgaaaatttaatggataaatcaacgatgcatgactatgaggtttttcaccgtagtaaatactaccaaggaaataatcgccttatgcaggcaaataaagagagaatgatggagattcaaccagatttagtggtagatttctgtcaatgtccacgtttattttttgcagagaaatacgctgatattctatctgacatggaacctttgggaatagagcgaattaagaacccgtaccgacctcacctagatgcagaagtgcagattgacataggtgatgaagaagaacacgagatcattgacgatgttgattatgtattcttccacgtatttaaccgctatgacgtattagataatgaggctactgtgtggaattgcgaaactccctctattggtgctgtcgtaactcctgtattggatgaaagtaagctccatgctataccccttcagcagcgattgatatttaaactcgatcaagacccctcatttatgttcgtgcatgatagtgttgttgatgccatgctcaatgccaatatgcaagatatctgggttcgttgggcagattgattttgccacttccaaattcagcgccgaaaggcgcttttttttgagcacatagcttcaatttatgtaatgtgttgagtattttgtaagaggagacgcaagctgaacgcttagccttctttttattatttgtttaagaagcatgatgtgatgccaattagtgaagaatattatgtgttatcaaatgactactcaaaggtgaattttgtagtaaacatggaagatgaacgtggatttagtttgttaatggataaagcgatatctacatccatgcatgttaactttgaatgtattagcttagagaattttagcgctatagatgctccatttgatgtttctgtcaacacggagatattatttgacctggattttcttgaagtgttatcagaattaacactatacaaaaattcacttgttccagctaagattaaagataaagagtttgtttatcttcattgttataatattattgatatagaggatgaagttaagggggttaactttgatctattatcagaaatacccatcgaggaaaggttggtatttaagccctcttatggggctatcgttgtttttttttcataaatcaattatagaaaaaaatcaactctgttaagagtttttcatattcaaagcatcaaaaggtttccgattggtctgttgaatgggaaatgtcatgaattatacagagcttaaagttgatgacctagctagcttggataattataaactccgacttgttaaagttgaaccggatatatatcccaaacatacgggctaagtgtctttataaatgattatgtcaaagggaaaatacgtcatgttgacgctctgaatttaactgagagtgttatatcaatccgttgttttaaaccagattgtgttttctataatataattattcgtatttacgattgttcttaacctggtttatgttataaaatttggagctttccacgtttaaaatgcaaatagcatacataaatgaggactttaacgagggtgaatcattatttactgccgttgatagtcggtcgtctttctatgatgataatgatttcactcccacttcttgtaaaagtgagattgtgtggaaagatgatgatatctatgaaattgaagcattagatttaatcagaggtggtgcttttaatgtcactaagcctattgctgaactaattatgagtttcgacccatacggcgttgaagtttatcccgcaaaattaacctgttccggagacggagtgttaactgagcgctacataatagcagtagataacttagtagatgttgttgattatgataaaggcataaaagtgactgacgtaactactcgtttttacttatctaatgaaaagatcaaggaacttccggatagcaagaggcacgtttttaaacctgtcggaatgacaaaaacgttcttctcaatcgagttgtttgaagcgcttgtccgtttagatgaggagggtaaaataaatactagtatcgttgccttttcatttgatacatccgaagagacgccccgagtctagtataagtaggtaaatataataatgactaactatatagttttatctaaacaatctttgactgacttcccattccagcaaagccctaagccaatagtgccagtagagccagatttgcttttggaaatgaccttttctccaaagctatttatcatcagtgatattgcgtcaaaagtagagaaactggtcgttcatggagtagagtggttagacgctcgcgtggattgttctccaagccagccatctgatgatgaaattaaagtttacgaagattatcgtatgccttacatccatcaaacttataaattgacggataaagaaaaacaatacggcaagttgaactggttagacatcgagtcaatagagtttgacttctctaaattagaaaatattcctttagaagaacgattaatatttaagctagaagaagactttggcttcattttcattcaccaaagtgtgattgaccttcttaaaaaagacgtcaaagacgtttgggtaagggatgtatagaaagtgccgaaaggcgctttttcttttaattacaatccaattactattcttaatagttgatgttatgtagataaaattcgcaatgggcttgtgggtaactgttcaattgtgaattcctaccaatttcacttttatctgaaatattgtcatcacagcagtcggctgaacaagtgaagtaccactt

SEQ ID NO: 3 - DNA, 453 bp, synthetic construct
  source 1..453: mol_type = other DNA
atgatttctaatatgactttacaaggcttagataagttagatcgcaaaatactttccagcctgttaacaaacgggcgtgagtctattgcaaatctctctagaaatatcggtttgtcccgtactgctgtcgcagaaaggatcaataggttggaaaaaaccggtattataaaaggctataccgcacaaattcggattgaaagtgacgggagaaaagctgctagctatttacttatctcatgtgaaaaaggaaagaaaaacgaagtaactaacgccctaaaggaaattcctgaagtacgtcttacgagcgtagttggcggcagcttcgatattattgctttgattgaagcaccagaccttcagtctattcaccacctttgcaacgaaattgagtcttttaatgggatacaaaaactagacactacggttgtacttcatcagccgatcagtcgatag

SEQ ID NO: 4 - DNA, 5207 bp, Vibrio parahaemolyticus
  source 1..5207: mol_type = genomic DNA
atggaacttaacattatgtttttcagcgacttcacaagaagaccgatcgatgaaagtaatccaagcggtaccaatccaaatggcttagacgactttgatgaaataaaacgtcaaataaatggcttgaacaaagtaacaggccgtgtgtcatggaagacagttcagacactatcaaaagaaatccttagaacgaaaggcaaagactttcgctgtagctgttactttactgtggcagccattaataatgacggactaaaaggcctagttgagggcttaaattctgtgctcgacttgtgtgttgtttattggttctccgcctatcctgagtacacaaaagctaatgcccgtattagtgcgattgaatggatggttgagtacactgaaaaacgaatgaagaaacaccgagtcctgccggaagaactccccctaattgaagcggctcatcagctttgtctacgtatcgaagaggaacttcgccttcactatggtattaaggcaccttcttttggacgtattcgtcgtcccctaaatcagtggattgaagagattaaagaagaaaaggcgaaacaagagaaaacctcacaaacaaaacctgcgtcaaatcctccttcagcaggcatccgagtcgaaatagcaccgcccaatagacctgttcaaaagaaagtagagcagcctaaagtggaagaagaggccaaatcaagccgattcattgtctatacaatgatcggattgctgatcatagcttttacgtctcacttcatatacaaacagcatcaatacacaatactcaaggaaagaattgggtcagcatcacttactgaattagtgacgattgttaactcgctcagtgtcgaaaacaaaaattaccaagatgacttgcgctctgtcacggttgagcgtcttcatactttgatgtcaggttggacattagatccagtcaaagtgagccaggtcaataccattgaacagttaacgaatgacctgaccaagctctatccagactcatcctctgcacaacaacttcgcgataattttttaggccaacgttcttattttgaagatgaatttgaggctatatacaaacgttttgcatatgctagaacggtctttgccaacattgcaaaacaaagtgcagacaaggacgctaagaaagcatatgagtacagcaattcgctatttccccttcttgggcgaattgaatatgcagaaaagaactctcagagaaaggaaattgaacgatcactccttttgctgaacgcttacctatataaaatcaaccagctacaagctgcagaaaaagacaataaataaaagtaaagaagttacaggcttatgttcaaacaaaaaatactaagaaatccagtcgtggtttctaccatagtggctttggtattagccgcgatatttgcagcggtatatttattcatacttgaagacgcagataagctttatctatgggttggtttaggagttattactggtttttattttacattccagctttccctatggcttaagaaaaaaaacagctcaaaagttcaacgtttagagacggaagaagaagctctttcaatggtgatccgccctcttctttctaatgcgggcaaaaagccaatctatctgatggttggtaataaaggggccggtcgacgtcagtttttgtttaattccagcgcaattaagccgatggaccgttctcgcacagcaaaaaacgatttcttcgaatggtatgaatctgatggtgccgtttatgttaagccagatcaacgcctcatcttccaggaagtttcaagcagtgatgcttctctctggaatacttttattgatgaggtgattcgtcatcgtccgagaaaacccttttcaggttgcttattctttgttgatttcgaattcatgattgtatctgagccagagcagaaagattacatcatgacagcgctattgcagcgtttgaactctatcactgagaagacctcgtctgcactacctgtttatttgatgatgacgaaactcgacaaactagatggctttaaagaatacgttcactctagttcactaaaaacacgtattgaatttctgtccattccattgaaggaagcgaaaggtgtacttaccgaatactacctagatagcttccataatctggtaaaagtgctagaaagcaacgctttagacgcatcggcaaactcaacagatatcaacgaaaagcaagccatccttgccttcccaaaacagtttgaactctgtcaacgtgaaatcagctacgtgcttgaacgtctttgcgatacaaacaatggcgtttactcgttggatattcgcgaaatcttcttctgttcttctttgcaaggtggacgcaagtacaacttgttagccaaaagctgcagtaattacttcaatttaccaattatcgcatctgagcacacgcaactaactgaaacaccgtacttttctcgcttcttgcttgactctcaaatcctcccagagagtgacttcgcaggcgagaacaagacttacctacgcatgattcagcgtcaaagccatttggcaatgctggcatccgttgtcttattggcaggcggtacttatttttttgtcacaacactagacagtaacttgcgtgttattaaccaattgctgaatatcgacgaaaaagttcaaacagaacaagaagcaagcttcaaccagttattgcagagtgctactcgtgctatccaaccctcatacaatgcttggttagaaggatcacgagcattagatgaagaagtaataccgctaaatataagcaggttaaacgaaagtacaaaattagcgtatgaagcattgattaaagaggttgctcaacaacttatgcctgttattgagcaaggctaccgttttgaactgacacacaaccaacatgatgtcacagcaacgttgccgttattaaaaggctacttgatgttgaaggatccgaccaagcgagatattcagtttttacgacgccaaacattgcaaacactgacagacttgagcgataagccggatgtggtaaatcaagctatgcgatacttggacgcctacttccgcactcaattctcacctgtagatatcaacatggatgtcgttcgagcaactcgtcgctcattgttggcaaattcaaatgtggatcttgtttacgcaggcattttaaaccaagcagatactatcgatcttggcacactcgatctacaacgtgcagttggctttgaatttaacaacgtatttaacgagccgttagatgacaaccgactagtaatcgataagatctatacctccactggctttagtaccttctatcgccctcgcgtcgacttgatgtctaaacaagtgatcagtgataactgggtgctaggattgtccagctatgttatcccaaccaaagaagagcaagaatcgtttaaaaatcaagtacgtaaaaagtacaccgatgattatatcaactactggcgcaacgctctcagtgagttaaaaattcagagttatagcaatgtgggtgatctaacaaatgctatcgacctaatctctggaccatcttcaccaatgacgactgtattaaagcaggtctatgcaaatactcagttttcaccggtaggagaaaaaaacgtactcatctcgaaagtagacccaaaactggcagaagtggcgaactctgcgtcagaagaggttgaagaagtggttcagccggattacttgttgatgaagcgtgtagaacaagcgttccatcttctaaatcagcttcaagtaagcgagactcctaactcccctactccatgggacgaaactatcgcagctttaagccgagttcgcacctacatgaaagatattgcggatgctcccgatccacaaatggcagcacttgcagccgctcaacaccgaatgagcagctctgaagccgatccgctaattaaacttaaacagattgctcagaagtcaccagagccagtacgcagttggttacttgatgtcgtaaagcaaagctggtcggtaatgatcaccgaatcagcaaaaggcattcaaacccaatggtatagtgaaatttacaccaaatttaaagagataggacttgataaatacccatttgatctcacagcaacagaggagatttctatcgaagacttcgaactgttgtttgcttcaggtggattgttagacacatttattcagaaaaacttcgcaccgttctacgataccaacttgtgggtaccaaaacaggtggatggtgaaacaatgccattatcttcagccttactggtgcaattgcgcaattacaacgttatccgcgacacgctgatcaataagagtactaaccgagtttatattccattcagtgcgaaagtattagatcttgactcaagtgctatcagagccagtttgaaaatctctgacactgatatcaactactatcacggtccgagccgaattcgtgaaatggagtggccaccgcaaaacggcgacttcaacattagtatcacgatccaagatgtgacagacgaaggtaaacaacacgttttaaataagagtggtcaatgggcgatatatagattgctaggtgattcgaccctgacaaacactcataacggtagcttcgttagtgacataaaagtatcagggagggatttgagcctccgtataactcccttgacacaaaaaaaccctttcactttggctgagttgtacaactttacgctcccagagtcgatttgataacaatataataaccagaataggcagaacacatgatttctaatatgactttacaaggcttagataagttagatcgcaaaatactttccagcctgttaacaaacgggcgtgagtctattgcaaatctctctagaaatatcggtttgtcccgtactgctgtcgcagaaaggatcaataggttggaaaaaaccggtattataaaaggctataccgcacaaattcggattgaaagtgacgggagaaaagctgctagctatttacttatctcatgtgaaaaaggaaagaaaaacgaagtaactaacgccctaaaggaaattcctgaagtacgtcttacgagcgtagttggcggcagcttcgatattattgctttgattgaagcaccagaccttcagtctattcaccacctttgcaacgaaattgagtcttttaatgggatacaaaaactagacactacggttgtacttcatcagccgatcagtcgatag

SEQ ID NO: 5 - DNA, 7706 bp, Vibrio parahaemolyticus
  source 1..7706: mol_type = genomic DNA
ttgtggagaattcaaatgcatttagtttgcaaatttatacctagctcgaagctttcctccaacgagctatcctacgtccttacacctgatgagtgtatagggcagctttcgcgtgtgcgaaattcagaagacattctcagaaaccttcctaaagagcttgctcagaagatctcaatctctgccaagaagtctagctccgggttgctaacggctattcgacacgagctaggaaatgggaactgggttgcgttgtcttctttctctcgtagaacgccgttaacagatacccaattacaaagtttcccaagactaaaagcacagcttgaatcggtctcatcgactggtgagagcaaagtttataaagcgggttataagcaggtaaaggacgacgtgactttggttcgtagctacacgcatgtcccttctgagccaagtccagaccaaaaaatcgttgtcgagtttgcagggcagtggtctagcaacgcggcatgtttgatgttgggaaaaaccgaagctcaaaaagaaaaagttactgtaggcaaggctgatacagaaaacaaacaccgcagcctcgcgacatttaaagatctcgaagctgagggcaaaacgctctatatcaaaatcccttgttccgaccagcctcagccaatcttgcttaaactcgccgaagacctgcaacccgttgataaagaaactcaaatggatgagtgggataacgtgttggtgcctgtggttccgctgcacttccctggtagcgataaatcagatgaagctgcagaagtgtttaagtcaggctacgtgtatgttgtttggaacaataaaatttggcgcgaagtggcaattacggaaaatggttacttttcagatacggacattaatagtgttcgtgaaggctctagacccaaaaggcacgccgatatctatatgaccaatcctgaaacaggcggtgttttcgcttatgagcctttccaaatcgtgcagaacggcaaggtggtaagtgaaggctctttgaatggctctggagaggctcgagtgttcaacttagttgaggaagaagttgagatagtcatgaccggctatgagccacaaatcaaagaaaagattgaaacaaatttgagccctattaatgcttctagcccagtggggcggagcgcgcaaggttaccctctcccgcatatatggcttccttacaagattaaaggtgagcctcaagaagtatatttggcttataacagtaaacgattgagcgagagtgaactatctgaactagaaagtgatcccggaaccaaggccataaaagtcactgatcttaaccattacagcagtgagaaaagctttaaaatgggagacggttctgttcgtctgctctcagtattaccaagtgctgcgacctcaaaacctgaaaaatacgcaatgttacgaagccagataaataaaaatgtagcggtcgtgtatttacttaaatctgtcgagatagttttcgaatatccgggctatacgacgctggacgagtcagatgattactttgagttaaggcaaagcgatggtgattggtcgcagcgagtgtgcttgcgccaatgtataaaaaaagagaatggttctaggttgattcgttttactggttggcctgcagaggttaaagaagtggatcttctccgtggttatcagggcaatagtcatcacggaagagataataaaacggtcatttttgctcaaacacccattgcggatttactggcttataagaaaaaagatcagccaagttaacaaaacaacttattcgagttgcgttgtcacactattaaacgcgatcgagaaagataacgaagagaatataagatgaatctacgtcatacattatgcttgattactttagcggtcttatctggctgtaatcaagaatcggccaaggaaccagaacaacacgcgccaaaacctcagacggttcaagaccgaatcgcagaaaaaaatgctcaacagtgggaagatggtcgtgtttatatcacagaaaacaattacgttatttatgacaaagagacgggtaaacctaaacactttatttttaaagctaaagaatggccacgtgaagatctcaccaatgaaggctcttatgatttaccacgtgtagtgtttcgttgggataacgcccgaaatgaagcgaaagaccactatgatcaaagggataaagtgtggagtatcaaaacggatgggacggacttacgtctggtcgcagatgagtttgttggcaaagttcgtctaatgcgtgtctctccaaataatcgctacttggctttggcctattcggcctcagaagggatgtttaaagtaattaaagatcttaagacgggtgagtatattgaactaggtcgttcacgtggctacccagagtttctatgggctgaagacagcagctatctttactacgtagacaagtttaaagactggaagtacacgttagcgacgggtgaaaaagaagaaacggaagttaattttaatgaatactctgttatatataatggaaaacgaattgttgttgttgcatatggagtagcagttttcgatgagcagacgaatgaactgctttattctgttgcacctaaaaaaagaggaggtgatttggatgccaaggagttccgtaagaaagcaattagcccaacaggacgttatgtttggtcagaaaccagaacgcaccgttatcttattgacgttaaaaatagaaccttccagtcggaagaaattgttccaagagcggtaggtaagttacgttattttgaaattctaggtaaagatgtaaattacacacgagatggagcggctagagttatattgagcaagacaacagaaaacaatgaatttggaccatacctaaaatgggagcaaattgggacaggccatagtgctggtaattcttctctatacaacgcttttgccaataatggtgattttgttaaagagggggatttatgagccttgtagagaactgcaatgcaataccgctacatgctagttattatttatttggcgcttactttgcagccgaagaagatgaaaataacgacaaaacttacccttcatggcactccaaaacgagtctagttggagaaaatatttccaatattcgcccgttagcgaaagtgccaatgcttaacgtgactgataatgaaaaccctaaactgtgggcaactaatcatgagtttaagttatgttcactgggtaacggaaagcccgatcaatatggggcttttgaggtcctgacaccagagcacacaattcatgctttaggtagggatctacccaaagagggtgagcagaccttcactttccgctttccgcccttgcctttaatagcttcactttctgagttaaaccccaaagagttagctacaaaagttgcagagttgagtagccagttggaaaacagcgaagcaaaactgatcttgacagagatcagcattgaacaattggctaagccttacgaaccagctgcgaacgggggactcagcgaagaacaggcagaatataacaagtggtatgaagatgtaagtgaatcgggagagacactaaaaggcttcgttttacctgttgtggcgagttgcgctgtagttttttctgatcaaggttatgaaagtgcgacggttactttgcaaaaatttgattcaaccaaagaaggtggggaatacaaaacaataggttcttcagtacaagcagcattacctgaagatgagcattcagaagaaaatgctaagcaatacccatgtgcgatgtttcacgttgatcctgctgattttaaagaaggcttatacagtatccgtgtcgaatttattccaagccagatgaaaaatggcgttccacagtttattaaagacgctcatattcagcaattttctgaaggtttagatacagtctacacttacgagctacgtgaacagattaccttttcttcaagcactccgttagggaaatttgctattgtcagtggcgatatgatctccctcgaggaatccttgatttatcaataccctcagtactttggaaatatgaaacattaccttcacggtaatgtacaaggtgcaactgccaatacgccagcttcaagaagtattgcgttactcaagaatatccgagatgtgtctgctcagctaggagctgggctgctgagcggcagcttgcaagacactctagaccgtgatggtcgccaagcagtgtttaataacgtctcgaaactgtattgggaagcagttaagtccgagatgccgcctgcgatgaaagcggcgggcgaattgtattacggtatttacagtacgaaagaagcgcttgataagttgcaggaactccacactccagctatcgcgggtattggttggaaggcgctatttcacgataaggtttttaacaccaatgaagctcgggcaagcgccctagctctttctttagaatcaagtctgggtaaccgacgtgtggttgccgggcgagttgcggaagcttggctttctggtggtaaaaccgttggttttaatggcttcggaacgggattgacagcacttagtttgtataacaaagctaaacagctggttaatcaaaataaaaaggttgataaagcagagcaaggaacagaagaagttacaaaagactaccttgctcaaattccagtttggaagacagctaaccatattcaggaagataaactcgaacaagctttaaaagatgcgcgtgatcaatctggcaatcagattgacgccattctcgtaaatagctctagaggcgcaggaataaaaatagaatttgcctttaattcgcgtgaaaaagagttaaaggatcatgcatctatatttaaacagcttgctgcttctcttgccaacgttttagaggatgagccaaaccttagagtagagattgaagggcatgcttgccaggtcggtaccgagaaagcaaacatgcaggtctcggcagagcgcgcagaatatgcgaaaaaactgctcatgaaggagtttcctgtttttgacggtcgtgtttcgatagccgcttttggtgaaagtaaaccggtctatgttcccaagcaaggtgaaaaagtcgaccgcaataaccctaatctaagacaaaaccgtcgagttgtgattcgtatttacttacagtctctgaacgtattctttcatcctagtcgctatggttcgcatacaatggaacgctctaggcttgctctagaagctgagatggttgcgcaagataaagcagaagtcgccctgcgaatggcaatttttgacagtgtcgttgacgtcgcttcctatattcctgctatagctcctgcggcaagagggtttatgcttgccatagagggttcaaagacagcaatatctgcggtgaaagtgattgatagtctaatgttggactccttcctagaagagttaaagcaaagccatgaggttgtcagtgagcttgaaaggctggcaaaaatacatatcgagttacttaacgatttgcgacgcagtaaggtagatgtaggtattgaatgtaggtcttatcaggaactcattactcaccttgagagcgaagaagttcgtaaggaggtgttaaaacgctaccaactacgagcgcttgctattaatggcttagtgttattactcgccgacctaggagctcaaaccaaaaggatttctaacatctcgtttaggttcttggttgagcgttacaaggtgaaggaatatattgagaattacattttgagcgatgattggtctgtacatacgatcaaaggaaacaccttagcggctaactggaagaaccgttgtgataacgagtactacaccgaattgtcgacaaagatgccacaaggtgcgcagcattggcctgctccaataacaactaaaagcaacgtttctcatctctctataaccgaaaacagggatagctttaaagcgtcaggtgcatttaatcaaatcttcccgatacaaactatgttgtttgaacacccaaatgaaacgatgtttgataactttaccgagtcatttaacccgcagggaagtgagttaaagaaagatgaaatcggctattgtcgaatacttgttcagtcagctcgttatactgaaagtgatgaaccagaatggatgtcatatgacagttggattcagcaatctagcaacagtcgcctaggtccatacgataaagtgaaagtgcagctggtacttaaaaaagaacacaagtatgcgaagccagttactattggctacgatcgcgtcgacgggctcgaaaatattaaaggtccagctttcagtgactggatgctgccaatgaaggttagtgacttcaacgctgatcctgatgggcgaataagtgctttttacaagcaacagggggttgatgacggcgctacgttgattgctattgagcatattccttcattccgcttcggtgatgtgatgatcgacggtatgaaaccaatgacctctaaagcaaagctaatgttctctgatgcaatgttcagtatggcagtttccggagcagcgatatcgggtcagccaagtgactattttgcagaagcggattcctttaaacgctatgtaaaaggcggaggcttccaaaatatgcgttactgcttgggaattagaacgaatcaagggcaacatagtttttatttaccacaagagtacagtctggggcagaagtttgatgatgctgaagatgagttgcaaattggcgctttaagtacggaaactagactggttttacttgatacctatttgggagagcagcaactgcgtctacgtgaacaggaccttatggtagagagctttaccttaatggcaagtgagggagacaaaaataccatccctatacttcatggcattaaacagcaagtggtggctatagacaccaaagcgtctggtttgaactttttcagtgaaagaaaatggtaccaatcggctgataatatctggcgtgatggtttctcatggggaaacaaaaagaaagacgatcctgccagtatttatgtgctgatttcgggtgaaagtcatgaaaaagcactttatgaacgtttgggtatgccgtgggaagcagtgaatatgtgtattcaactggcgctcgacggaaatgatggcaagccagttaaaggtcctctttacttcaccgatatgtatcatattggtgagtttgcccataaaaacgaagaatggatgtttactgaatctatttcagaaaaccatcgagaacaagttgatgccatgagtgagtttataggtgaagcagtaagcagtctcactaaagagaatgatctatctaagagcaaaaactatacgatgtactgcatgaggtttaacttggaatatgtcgcaccaacgggagttaggttaaaaggcctaagaccgtttggtggtgtaattggtggtaaagcgaacttggagctttcggttgctcatctaaaacaagtcggattaaatggagttgataactatgatattggtcgtgatgctgtaatttctattccttctttaaatgcggatagtgctttggcaggtaatttctttagtgatatgccttggctaattgagaaagagtcgacagaggaaggcgtagacaagtctgcggctgaaacttggaagcagtataacgaatctcaacgcaagaaatggctgaaagattggattgagaaacaacctgcggtaactcaagcacccaagcccttggaaaagtcacttgataacccttcctag

SEQ ID NO: 6 - DNA, 2205 bp, synthetic construct
  misc_feature 1..2205: note = T6SS1 antibacterial effector-encoding gene: vp1415 includingmutation in the catalytic AHH to AAA
  source 1..2205: mol_type = other DNA
atgcgatctcttttattattcaatcaacaaaattcaaatggattggttatgggcgtaacagttggtgcaaatggactttcgatagttcataaaggctcaggtggtgaagcgaatgcaacgctcccagatgtttgtttaacgaaggtcggtaagccgatcgttcctatcccttatggcaacaatgccaaatcagcagatcttgctggtggcactacgactatctctatggatggtggcaacagtgtcgcaattaaaggcagtacgttttcaaaaagtaccggtgatgcgggcggcgataaaaaaggcgtcgcatccggcacgatagaagccgaagcaaaattcatttcagcctcaccaacggttaagtttgaaggcaagggcgtatgtcgcctgtctgatcaaatgaccatgaacaaagccaacactatgtgtttgggcggcgcacaaaacccatcggtttctgttacggaagcagatgatggaacatttaccatagacattgattatcgatacccagatggagacccagtatacaaagccgatttcaagattattgacagtactggtacagaacatgttggcactttaagcgacaaaggtaaggggacgattacgggcttaccagcaggttctatcagtttattcctgggtgaggataagagagatatagagccaatccaaaagccgcaaccaaacaaggattatcttgaactgccggggcctttggacttagtagagtttgctggacgtggcttggttgaatattgggatgctgttgaaatgccgagtgattatagtcaatgggcttggggagcgatcatgggggacttcaatcaagatcgatctgctggccaaattgcttttgatagtgctgttacagctataccattcatcgatcaaattggggacggtcgagacattagtgccaacctttatgctttttataatgcagacgacataagagaagataaagaaaagtacacagatctcgttatcactctagttggctttattcccaccgcaggtagcttgataaaagggttatttaaggaacttaaaatcttgggcaaagccgctgatttagatatgattgctgctttccttagagccggagcaaaaggtgatgtggtcaaatggatgcaaagccttgatatgtccgtcatcaaaaatgatatatatggtcaactagatgatattactaaacaagtaaaaacgttaatggatacacttgagcaagagagccgtaaaagagggtatactgttgttgctgacgcctataaaaggtgtgtggaacagctagatgacttttttaagaaagctgacggaccaatatctaaagtcttaagcgattttgatgataggctcaagatgatcctgcctgaggcacctttagttacatcagggacttcctttagtatttctgcagggacggctcaaggaggaggcaaagctgaggtaattaagactcgtaataagacgaaaaagaagactgatacgtgtccgttgtgtaataaaaagattggaaaagggaagaaagagtgcgaaggggcgaaagtcggtagtatcagcaaagcagttatggacgatggttcctctcaaaaattatggaaagaattcgaaatacgaaatggttggcgagacaagacagagcacccttggtggcacggggcgggagcagtgcaggctgcagctcttattcctaaaaatgcattcaaactaaaaaatgcaaagagtaagcaaactaaagaaaaaatgatgtttcttagacgaattgctcagacgtgcgcttacaacatcgacttttggaaaaatggtattggccttcctaataagaaagaaacagcgtgttttttgaggaaacctcgacatgcaggcggtcacgatcgtaatgaatttaactacacgaacgaatgtgtaaagcttgttcatgacaagttaagctcagatatcaagaaaagaacaaaagaaggtagttgttccaagacaacaaactcttcgttgattatgaagttcaatgaatcaagcaaaaatatttttgataatattgctgactttatctggtttattactcgtgatggaaaaaactatgatcctaaagtagaaccttacgtgggctgtagttcaaattgcaaaaaaatacacccgattattaatataaacactaagcgacaaattgaaaactatgacttagagattggaaaataa

SEQ ID NO: 7 - DNA, 1426 bp, synthetic construct
  misc_feature 1..1426: note = Exogenous effector and immunity pair Tme/i1 BB22OP - C-terminallinker + Myc-His6 sequence
  source 1..1426: mol_type = other DNA
atgagatatgaatccgctcctgtagccccaaaagaaagacaagaaacaactagacagcgagcagctcgccaacgtagagagcgtcaggaagagttgaggtataccgcgagcgatgaaaagcgatgggcagaaattagagagagggttcttagagatagacaagaggctggattaggtcaaatctctcaaggtttaaaaacgcctcaaattctaagcgattctatcgggctacagtgttcacttgacgatgaaatggcgaagaatcatcagcatgtacttattactgatttagaagatgcgcacaagatattgcataacgtttggctagattctggtaaagacgttgcagctcttgcagcgagtattaatacagcaaaatcatggttcaaccacacagagccgatgtttgacgccgcgtctattgctaagcaattcggggatatgaacattaaggcggatcttgttgagagcagaggaaaaatctttgttgcattcaatgggacagatcagaacgggaaagtactcaaacatgcgttagtgaatggctctcgtatcaacatgaacggaaaaaaatacccgcttaattcatttaaagcaatgcaaactggttttagcccgaaatctagggttgcaaactttaaaggtgcaggagttttaacgtttgttgtatcggcgagtattgctactagtgacctagtgttcaaagatgattaccacctagtggattggtttgggaatgttggctctgacatgttcaaatttatgctccaaagcgcagttggtgaagccgcattgtttgctgctgctgtcttaggtcaaccgataattatcggtgcaattgctgtaacagcgacatatcttctaattgaatgggcttggggcgaatacaaaatttctcaaacgatagttgaaaggttagaaagtgcgatataaaactaaaatccgtttggctggcttattttttgtttcactaatgctgttgtgttggttttatccttcttatggcgtttatttggattaccttgagctattgaatcgagccgatactgtgcaagtttccgttattaccttgtgggttcctgttggtgtttttggggctttagtgtgtgtgctttttatgacacctaaagcattgctatatgggaagaagattaatgaactttattctcaaagagctatgcagttagctaacaaaattagtatttattttgcattggcaggcgtggcatttgctgctggctggacatatcactctatcgacttgttagaaaaatatgggtacgtatacagccgagatttaacaaaaatcactccaacaggagtgcacttgatgtacgttaaagctcgaaagcttgggcccgaacaaaaactcatctcagaagaggatctgaatagcgccgtcgaccatcatcatcatcatcattga

SEQ ID NO: 8 - DNA, 2971 bp, synthetic construct
  misc_feature 1..2971: note = Exogenous effector and immunity pair VPA1263/Vti2RIMD 2260133 -linker + C-terminal Myc-His6 sequence
  source 1..2971: mol_type = other DNA
atgtcctataaagttcttccactgtcggaagtaatgtctcatgaatttggattgatagagggcaacttccataatatttcggaagcggatatcgcctctgctattccttcacatcttacttttgaacaactaaagcagcatctctacgaaggtaatctcgcactcgttagtgatacgcctcaaactcctgctctgcttagttacaacgacccaataggtcctaaaacttggcggctgaattctgaggttatctctgaactttcggatgatgcggctaataatttgttagcaataacgaaaaccaccagagcaacgggaggttaccgctcttgtatcggggaaacctcgacgctcgaacgagtttacacgccgcagccaatcgcaaccgaaagtgaagaggaaatcaccaaggaatttgagtacagctttgaagttggctgctcggatgccaccataaagaaaatggtgcacagtgactttgctttagcgaaaactgaaaaagagaacgcggtaacccgctgggaacaaaccaataccgagcaaggcacccgctacactgcgttatgtgtatttgatgagccaaagcgtttaaatatccacatagctgatgacaatctcgggctgacgccactggaagccgtcacattacaaaaagccggcagctgtaaaacggacgaaggttttatacctgttgtccctgcagtgcgattgggcgaacgtttaggtttgcctacagaaggttattactaccactttaatgatggcgaacttgtccaagaatacaaaattctaggagaagaaaagtgggcgttttacgcaacgcaatcgacacaagacaagctcaatgatgagcgtggttttaccaaagaccaaagcgcaattttggtgtattggaaattagctgatcaaatcatcgagaatcaatacatcatttatcttgagcgccaaatcacccgagaagagcttgataatctaagtgaagattggctgtgtgaaaacgggataaagttggatatccccgcattgtttgatgcggtcaaacagccggaagaagcgcgctcagaaggtaacaacgatgagacgcaagcggaacaaaccgcagcgacgcacaccgtaacggtaggggaagactggcaaagcatcgctgaactatatggtatgggagctaaagccttgctcttgcttaacccagtgtttgaggccgaccctctgtcacttgcggtaggtgatgaaattattgttgctgagaaacagcaacaacaagcaccggacaaaaagaatacctttccgccgctgcgcccacaaacttacaacaacatccgcaacagtcattatcagcacagtgatccattgctcgggttgaccaaatatagagccattaacactgccgattgcatagaaggcgatgtggtaatcctcaatttaaaagacgccacaagctcgatcgtgttcgctaaatcctgcaccagaccagagggatgtattgaaattggcgaccaacaagagtcaataagtaactttggtccttggtcattcttctttgcacaagccaatgcaaacccagctgctgtcatacccgccattcaagcgacacaagcacaaatggctatgggcagttccgcagctgtcgccggttccccagagcaaatgcagcaaacccaaaccgcagcgatgcagttagataaactggccggcacattgaaagaaaaaatcgtcgagggctaccgctggcaggtcgaaggtattggcgctttgtttgccatgcagcaaagcttatttggcgataacacccaatacacagaccaagatttacgccaagtaaccacggcgcaaagccgcgttcgtgtccacattaccgagccccaaggtggtgagttttatccgcacgtacagggctatcatgtagatgacacacgaatccccatcaaatatgtgaaacaagggaacaatggtcagttgtccgttgcgattgaagagaacggaccaaccatctattggacaccagaagaaaacggtgaagcaagttggcaaactacccccgatcatagtgatgggtttgaaaaagacgatatacttgtcacgccaattcactcggatagtgatgcaaacgtcaccgttacgcctgcgccagaagagaaagactggcgcgatgcgatcttggtattccctgaaagctcaggtattgctccgctctatgtggtttacaaagaaagcccgagagataagcccggagtagttactggtaagggcgaagatatctttggtatctggttagctgatgcgggtaaagacttgggtgctccaataccttcccaaattgctgataagctaagaggaagagagttcagtagctttgatgcatttagagaaggcttttggtacgccgttggtgaagaccaaactttaatcaaccagttcagccgtgcgaatcaacgtttgatcaagagaggccgttctcctttttcgctaccaagtgaacaagttggagggagacaaaggtatgagttacaccacaaagaagagatacagtatggtggagaagtattccatgtagataatttatgtgtccttaccccgaaacgacatatcagcatacataaggatcaataatatgaagcttgtaattaaagatgccttacaagactacaccgaggcggaattctatcgcttcttagaagaatttttcgaagataccgacactaacgacttagatgatgaagcttatgacttgcatatatcaaatctatccgctcatttttcagagttagtatctcaccctgatggtaatgctctgatcttccacccttccccaggtagggaggactctcctgacggtgttattttagaaataaaagagtggtgcgctaagcaaggagaaccttgcttcaaagagaagcttgggcccgaacaaaaactcatctcagaagaggatctgaatagcgccgtcgaccatcatcatcatcatcattga

SEQ ID NO: 9 - DNA, 3307 bp, synthetic construct
  misc_feature 1..3307: note = Exogenous effector and immunity pair Va02265/012G01
  source 1..3307: mol_type = other DNA
atgaaagtatatgatatgccaatagaattccctaagccgaaacggttaacaagtgctggatatgccccagttcaggaggatacccctctcgctcccccaactccaaccccagcgctcgaacctgagcctgtagcagggtttgagtgccgtattaagatctatgcaccaaagtccgatctctctagcctgaaattaggtgtcttctctcttagtaaatctgataatgaggagagcattaaacgctggcactctgaagaggcgttagactcggcaaagtataccgtgctcacaggagagtgccatgtcgaagaatcaaaaaagctgactcatgagtttcatcagaattttggcatcagcacatcctttgacgttacgccaagatctgtcggctcagggtactttaatgctgagtttatccccttgttgccatctctagagagcgagtctaaattaggttggtctttgcatggcttctattactacttctacgaaggtaagttgttgtatgaactcgatatactaggagacggcaagtggggttggcatattactcaatcttcatctgatggcatatctaacaccctcgcaagcaatcagatttttaattctctactggttccagtgaaaattgaaggaaaagaaccagggaaacaacacatactctattcccgtgctaagttgaccaacgaacaactgggcgaagtgagtatggcttggctcgacgaaaatgcgacactgattgatacggctcttcttgtttcttctttacaacagcatcttctaccaagagaaacaccttctgatatcaacgataccgctgccaaagaaattcaaacccataaagtgaatatgaaggataatggacagcatgaaagttggagtgacattgcagggtattacgaccttagcccgaaagaactcctcaaattaaactcatcattcgaacaagatccgatggctttgagagctggcgatgttcttattgttgaaaaatcggatgttgtcgatgaagctcaagaaaccaaagaatctccctcaccgttaacggagataaaagttggcgagagctatgtattaggtaatacatgggggaagtacaatcagccactgctttctccgtcaatgaaacacatctttgagcttcaaggtgtttctcagacaacaccagtcctcaatgtaaaaaaagtcgaagaaaaagtactacggattggggttttctttgatggaactgggcaaaaccatctgaatgatctctacaaagaaacacgtggagtaaaatcacgcactaatgtggcacggctatttgaaatttacccattagatgatgataccaataaaatttatattgcaggggtcgggactgtggatggagcgtggcaaaccccagccttgatagatgaaggaaaagatgaggtcgatctagctcaggcgtttggtgtcagccccaataatttagagaaaacatggacgttcattgatagggcttataaccttgtcgctgacgatactggggcattttataagtggcaaaattggacaaaacaattttatggaattatcactgatttgatccgccagcaaaaataccaagctataacacatattgagttcgatgtatttggttttagtcgcggagcggctttagctcgtcactttgttaatgccgttaaagatgggttacctgattataataagccgcgtgatggagagtcattcggtgaagttttcccaaacttattaacacaccaaaatgatatcagtagccagcttaaacatggttattatcctgacgagacacgtacctgtagtgttcgctttgttgggttgttcgataccgtaggctcgttctatctagcaggaaacaacgatgaaggcaattttgaactaggacttgatactagttgtgcggagcgtgtattccagataagcgcccatcacgaatacaggaaaaacttccctctaacgtcactgctacctagcgatagcattctaccttctaacttttatgaggaggtgtttgctggctgtcacacagacattggtggcggttatccttcaaaagagcagtatgcaaagcaaggtttacctgaaaggtatggaatgcctctgtctgcaacttataatcgtgagttaataaaaacagaatccataagggaagatatacaagcacaacagaatgtttatggagccgccgcatacagtgagcgcctacacaagcaagctcaagatgcttggaatttggaatgcagaactaagggtatatatggtgttgtgacactggtgaatggcatcctttattattacgagttacagcctattagcaatgctctttcggctttgccattagaaagaatgaaacagcaggcagaggttgcaggtgttaattgggatgacgaaaagctcgcctatcaacaagattttagtcaattgacaggaaatgatgtggctttaaaaaccttgaatgaaaagttattagctcaaccgttaggaagcattgcaccaacacattggcagggtgagattagccagtatggaaaggaatggatacatcggcctcatgatgcactaatcaacgcggggtgtgagactgctatggacaatcttgttaacgcggtaacgaaagaaggaaaccaactcaaacgagtggtatttgataatgaagtatagatttttaattgctgtaagcatgtgctttttgcttaatggttgctttgagagtaagccagtttcagttgcgagcattggcaatgcagggtctttaagcggtcctcattttattcgtgatatgtgggtcagtgatatatatggtggaggtgcaaactttgcgtatgggagcgttcagggctgttgtgctagcgcaagcattggcattggtatccctagcgccataaaaggtaaatgggaaaatggagttggtggcggctcatcccgtgaaaagtggcaaaagcgttggaagcccgaatggggtaatttcgatgagaattatgatactaaatggtttcgaattgatgatgcgatagatagtgagcttgccaagaaaaaaattgaaacaatgaaccattactacaagaaccatcatgcaaacccagtcatgaagcttcttgtaaataatgatagggtcttacttttgtacgcttacggctgtactccagaaataaacgatgactgcacggtcagagagggagcagaccctaatggatgggttcaagtagcaccgtatagcaagcataagaactcaactgtggttgttctttatgaaggcagaggtgaggtctcagacaaaccatttacggataatacaccttag

SEQ ID NO: 10 - DNA, 879 bp, Escherichia coli
  source 1..879: mol_type = genomic DNA
atggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataa

SEQ ID NO: 11 - DNA, 2283 bp, synthetic construct
  misc_feature 1..2283: note = cassette including the regulator AraC, the AraC-responsivepromoter, and the vp1407 gene
  source 1..2283: mol_type = other DNA
atgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgatttctaatatgactttacaaggcttagataagttagatcgcaaaatactttccagcctgttaacaaacgggcgtgagtctattgcaaatctctctagaaatatcggtttgtcccgtactgctgtcgcagaaaggatcaataggttggaaaaaaccggtattataaaaggctataccgcacaaattcggattgaaagtgacgggagaaaagctgctagctatttacttatctcatgtgaaaaaggaaagaaaaacgaagtaactaacgccctaaaggaaattcctgaagtacgtcttacgagcgtagttggcggcagcttcgatattattgctttgattgaagcaccagaccttcagtctattcaccacctttgcaacgaaattgagtcttttaatgggatacaaaaactagacactacggttgtacttcatcagccgatcagtcgatagaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttcta

SEQ ID NO: 12 - DNA, 7037 bp, synthetic construct
  misc_feature 1..7037: note = cassette including the regulator AraC, the AraC-responsivepromoter, and the vp1409-7 3-gene operon.
  source 1..7037: mol_type = other DNA
atgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggaacttaacattatgtttttcagcgacttcacaagaagaccgatcgatgaaagtaatccaagcggtaccaatccaaatggcttagacgactttgatgaaataaaacgtcaaataaatggcttgaacaaagtaacaggccgtgtgtcatggaagacagttcagacactatcaaaagaaatccttagaacgaaaggcaaagactttcgctgtagctgttactttactgtggcagccattaataatgacggactaaaaggcctagttgagggcttaaattctgtgctcgacttgtgtgttgtttattggttctccgcctatcctgagtacacaaaagctaatgcccgtattagtgcgattgaatggatggttgagtacactgaaaaacgaatgaagaaacaccgagtcctgccggaagaactccccctaattgaagcggctcatcagctttgtctacgtatcgaagaggaacttcgccttcactatggtattaaggcaccttcttttggacgtattcgtcgtcccctaaatcagtggattgaagagattaaagaagaaaaggcgaaacaagagaaaacctcacaaacaaaacctgcgtcaaatcctccttcagcaggcatccgagtcgaaatagcaccgcccaatagacctgttcaaaagaaagtagagcagcctaaagtggaagaagaggccaaatcaagccgattcattgtctatacaatgatcggattgctgatcatagcttttacgtctcacttcatatacaaacagcatcaatacacaatactcaaggaaagaattgggtcagcatcacttactgaattagtgacgattgttaactcgctcagtgtcgaaaacaaaaattaccaagatgacttgcgctctgtcacggttgagcgtcttcatactttgatgtcaggttggacattagatccagtcaaagtgagccaggtcaataccattgaacagttaacgaatgacctgaccaagctctatccagactcatcctctgcacaacaacttcgcgataattttttaggccaacgttcttattttgaagatgaatttgaggctatatacaaacgttttgcatatgctagaacggtctttgccaacattgcaaaacaaagtgcagacaaggacgctaagaaagcatatgagtacagcaattcgctatttccccttcttgggcgaattgaatatgcagaaaagaactctcagagaaaggaaattgaacgatcactccttttgctgaacgcttacctatataaaatcaaccagctacaagctgcagaaaaagacaataaataaaagtaaagaagttacaggcttatgttcaaacaaaaaatactaagaaatccagtcgtggtttctaccatagtggctttggtattagccgcgatatttgcagcggtatatttattcatacttgaagacgcagataagctttatctatgggttggtttaggagttattactggtttttattttacattccagctttccctatggcttaagaaaaaaaacagctcaaaagttcaacgtttagagacggaagaagaagctctttcaatggtgatccgccctcttctttctaatgcgggcaaaaagccaatctatctgatggttggtaataaaggggccggtcgacgtcagtttttgtttaattccagcgcaattaagccgatggaccgttctcgcacagcaaaaaacgatttcttcgaatggtatgaatctgatggtgccgtttatgttaagccagatcaacgcctcatcttccaggaagtttcaagcagtgatgcttctctctggaatacttttattgatgaggtgattcgtcatcgtccgagaaaacccttttcaggttgcttattctttgttgatttcgaattcatgattgtatctgagccagagcagaaagattacatcatgacagcgctattgcagcgtttgaactctatcactgagaagacctcgtctgcactacctgtttatttgatgatgacgaaactcgacaaactagatggctttaaagaatacgttcactctagttcactaaaaacacgtattgaatttctgtccattccattgaaggaagcgaaaggtgtacttaccgaatactacctagatagcttccataatctggtaaaagtgctagaaagcaacgctttagacgcatcggcaaactcaacagatatcaacgaaaagcaagccatccttgccttcccaaaacagtttgaactctgtcaacgtgaaatcagctacgtgcttgaacgtctttgcgatacaaacaatggcgtttactcgttggatattcgcgaaatcttcttctgttcttctttgcaaggtggacgcaagtacaacttgttagccaaaagctgcagtaattacttcaatttaccaattatcgcatctgagcacacgcaactaactgaaacaccgtacttttctcgcttcttgcttgactctcaaatcctcccagagagtgacttcgcaggcgagaacaagacttacctacgcatgattcagcgtcaaagccatttggcaatgctggcatccgttgtcttattggcaggcggtacttatttttttgtcacaacactagacagtaacttgcgtgttattaaccaattgctgaatatcgacgaaaaagttcaaacagaacaagaagcaagcttcaaccagttattgcagagtgctactcgtgctatccaaccctcatacaatgcttggttagaaggatcacgagcattagatgaagaagtaataccgctaaatataagcaggttaaacgaaagtacaaaattagcgtatgaagcattgattaaagaggttgctcaacaacttatgcctgttattgagcaaggctaccgttttgaactgacacacaaccaacatgatgtcacagcaacgttgccgttattaaaaggctacttgatgttgaaggatccgaccaagcgagatattcagtttttacgacgccaaacattgcaaacactgacagacttgagcgataagccggatgtggtaaatcaagctatgcgatacttggacgcctacttccgcactcaattctcacctgtagatatcaacatggatgtcgttcgagcaactcgtcgctcattgttggcaaattcaaatgtggatcttgtttacgcaggcattttaaaccaagcagatactatcgatcttggcacactcgatctacaacgtgcagttggctttgaatttaacaacgtatttaacgagccgttagatgacaaccgactagtaatcgataagatctatacctccactggctttagtaccttctatcgccctcgcgtcgacttgatgtctaaacaagtgatcagtgataactgggtgctaggattgtccagctatgttatcccaaccaaagaagagcaagaatcgtttaaaaatcaagtacgtaaaaagtacaccgatgattatatcaactactggcgcaacgctctcagtgagttaaaaattcagagttatagcaatgtgggtgatctaacaaatgctatcgacctaatctctggaccatcttcaccaatgacgactgtattaaagcaggtctatgcaaatactcagttttcaccggtaggagaaaaaaacgtactcatctcgaaagtagacccaaaactggcagaagtggcgaactctgcgtcagaagaggttgaagaagtggttcagccggattacttgttgatgaagcgtgtagaacaagcgttccatcttctaaatcagcttcaagtaagcgagactcctaactcccctactccatgggacgaaactatcgcagctttaagccgagttcgcacctacatgaaagatattgcggatgctcccgatccacaaatggcagcacttgcagccgctcaacaccgaatgagcagctctgaagccgatccgctaattaaacttaaacagattgctcagaagtcaccagagccagtacgcagttggttacttgatgtcgtaaagcaaagctggtcggtaatgatcaccgaatcagcaaaaggcattcaaacccaatggtatagtgaaatttacaccaaatttaaagagataggacttgataaatacccatttgatctcacagcaacagaggagatttctatcgaagacttcgaactgttgtttgcttcaggtggattgttagacacatttattcagaaaaacttcgcaccgttctacgataccaacttgtgggtaccaaaacaggtggatggtgaaacaatgccattatcttcagccttactggtgcaattgcgcaattacaacgttatccgcgacacgctgatcaataagagtactaaccgagtttatattccattcagtgcgaaagtattagatcttgactcaagtgctatcagagccagtttgaaaatctctgacactgatatcaactactatcacggtccgagccgaattcgtgaaatggagtggccaccgcaaaacggcgacttcaacattagtatcacgatccaagatgtgacagacgaaggtaaacaacacgttttaaataagagtggtcaatgggcgatatatagattgctaggtgattcgaccctgacaaacactcataacggtagcttcgttagtgacataaaagtatcagggagggatttgagcctccgtataactcccttgacacaaaaaaaccctttcactttggctgagttgtacaactttacgctcccagagtcgatttgataacaatataataaccagaataggcagaacacatgatttctaatatgactttacaaggcttagataagttagatcgcaaaatactttccagcctgttaacaaacgggcgtgagtctattgcaaatctctctagaaatatcggtttgtcccgtactgctgtcgcagaaaggatcaataggttggaaaaaaccggtattataaaaggctataccgcacaaattcggattgaaagtgacgggagaaaagctgctagctatttacttatctcatgtgaaaaaggaaagaaaaacgaagtaactaacgccctaaaggaaattcctgaagtacgtcttacgagcgtagttggcggcagcttcgatattattgctttgattgaagcaccagaccttcagtctattcaccacctttgcaacgaaattgagtcttttaatgggatacaaaaactagacactacggttgtacttcatcagccgatcagtcgatagaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttcta